(19) United States
(12) Patent Application Publication
(10) Pub. No.: US 2022/0041627 A1
(43) Pub. Date: Feb. 10, 2022

(54) PRODRUGS OF ANTI-CANCER AND ANTI-AUTOIMMUNE DISEASES THERAPEUTIC AGENTS, AND METHODS OF MAKING AND USE THEREOF

(71) Applicant: UNIVERSITY OF MARYLAND, BALTIMORE, Baltimore, MD (US)

(72) Inventors: Fengtian XUE, Potomac, MD (US); Yan SHU, Clarksville, MD (US); Yong AI, Baltimore, MD (US)

(21) Appl. No.: 17/311,216

(22) PCT Filed: Dec. 4, 2019
(86) PCT No.: PCT/US2019/064471

§ 371 Date: Jun. 4, 2021

Related U.S. Application Data
(60) Provisional application No. 62/774,993, filed on Dec. 4, 2018.

(51) Int. Cl. C07F 5/02; A61P 35/00

(52) U.S. Cl. CPC C07F 5/025; A61P 35/00

(57) ABSTRACT

The invention relates to prodrugs of anti-cancer and anti-autoimmune diseases therapeutic agents, and methods of making and use thereof.

### STATEMENT OF GOVERNMENT SUPPORT

This invention was made with government support under Grant Number GM099742 awarded by the National Institutes of Health. The Government has certain rights in the invention.

### FIELD OF THE INVENTION

The disclosure relates generally to the field of reactive oxygen species (ROS) activated prodrugs of various anti-cancer and anti-autoimmune diseases therapeutic agents, including but not limited to prodrugs of 5-fluorouracil (5-FU).

### BACKGROUND OF THE INVENTION

The FDA-approved chemotherapy drug 5-fluorouracil (5-FU) was first introduced by Heidelberger et al. in 1957 as an antineoplastic anti-metabolite of the uracil anabolic pathway. In the human body, 5-FU interferes with DNA synthesis by blocking the activity of the nucleotide synthetic enzyme thymidylate synthase (TS), which catalyzes the conversion of deoxyuridylic acid to thymidylic acid. As a single agent or in combination with other chemotherapies, 5-FU has been widely prescribed for a variety of solid tumors including breast cancer, colorectal cancer (CRC), stomach cancer, pancreatic cancer, and cervical cancer by injection, and skin cancer as a cream. One of the marked limitation of the activity of 5-FU is its rapid degradation into 5-FUH2 via the action of the cytosolic enzyme dihydropyrimidine dehydrogenase (DPD) which deactivates more than 85% of the injected doses of 5-FU. Further, the bioavailability of oral administered 5-FU is highly unpredictable. Even though 5-FU was first introduced in 1957, it remains an essential part of the treatment of a wide range of solid tumors. There remains a need for more effective and less toxic 5-FU forms for the treatment of advanced diseases. To date, there have been no reports of ROS activated 5-FU prodrugs that have successfully entered the clinic.

### SUMMARY OF THE INVENTION

The disclosure provides a compound of Formula I, or a pharmaceutically acceptable salt, solvate, hydrate, cocrystal, or prodrug thereof.

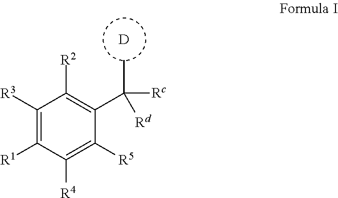

wherein in Formula I: D is a drug or prodrug moiety comprising at least one heterocycle; R¹, R², R³, R⁴, and R⁵ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted alkynyl, substituted or unsubstituted heteroalkynyl, substituted or unsubstituted cycloalkynyl, substituted or unsubstituted heterocycloalkynyl, substituted or unsubstituted aryl, substituted or unsubstituted heteroaryl, hydroxy, halo, cyano, trifluoromethyl, trifluoromethoxy, nitro, trimethylsilanyl, —OR^{a}, —B(OR^{a})OR^{b}, —SR^{a}, —OC(O)—R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{b}, —N(R^{a})C(O)R^{b}, —N(R^{a})C(O)N(R^{a})R^{b}, —N(R^{a})C(NR^{a})N(R^{a})R^{b}, —N(R^{a})S(O)ₜR^{b}, —S(O)ₜR^{a}, —S(O)ₜOR^{a}, —S(O)ₜN(R^{a})R^{b}, and —PO(OR^{a})(OR^{b}), wherein at least one of R¹, R², R³, R⁴, or R⁵ is —OR^{a} or —B(OR^{a})OR^{b}; R^{a}, R^{b}, R^{c}, and R^{d} are independently selected at each occurrence from hydrogen, substituted or unsubstituted alkyl, substituted or unsubstituted fluoroalkyl, substituted or unsubstituted carbocyclyl, substituted or unsubstituted carbocyclylalkyl, substituted or unsubstituted aryl, substituted or unsubstituted aralkyl, substituted or unsubstituted heterocycloalkyl, substituted or unsubstituted heterocycloalkylalkyl, substituted or unsubstituted heteroaryl, or substituted or unsubstituted heteroarylalkyl; t is 1 or 2; wherein any of R¹, R², R³, R⁴, R⁵, R^{a}, and R^{b} can optionally be linked together.

In some embodiments, the disclosure provides a compound of Formula I, wherein R¹ is —OR^{a} or —B(OR^{a})OR^{b}. In some embodiments, R² is —OR^{a} or —B(OR^{a})OR^{b}. In some embodiments, R⁵ is —OR^{a} or —B(OR^{a})OR^{b}.

In some embodiments, the disclosure provides a compound of any one of Formula 11, Formula 12, Formula 13, Formula 14, or Formula 15:

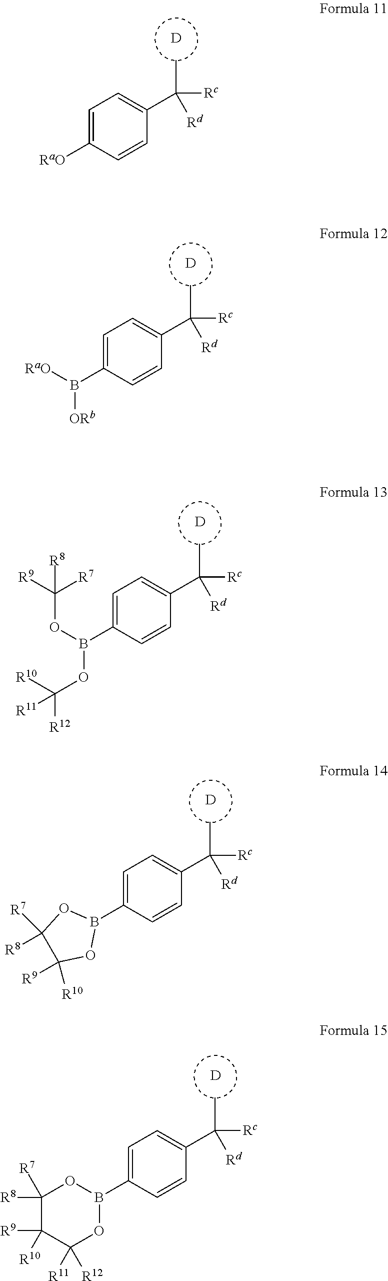

wherein in Formulas 11-15: R⁷, R⁸, R⁹, R¹⁰, R¹¹, and R¹² are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted alkynyl, substituted or unsubstituted heteroalkynyl, substituted or unsubstituted cycloalkynyl, substituted or unsubstituted heterocycloalkynyl, substituted or unsubstituted aryl, substituted or unsubstituted heteroaryl, hydroxy, halo, cyano, trifluoromethyl, trifluoromethoxy, nitro, trimethylsilanyl, —OR^{a}, —SR^{a}, —OC(O)—R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{b}, —N(R^{a})C(O)R^{b}, —N(R^{a})C(O)N(R^{a})R^{b}, —N(R^{a})C(NR^{a})N(R^{a})R^{b}, —N(R^{a})S(O)ₜR^{b}, —S(O)ₜR^{a}, —S(O)ₜOR^{a}, —S(O)ₜN(R^{a})R^{b}, and —PO(OR^{a})(OR^{b}); R^{a}, R^{b}, R^{c}, and R^{d} are independently selected from hydrogen, substituted or unsubstituted alkyl, substituted or unsubstituted fluoroalkyl, substituted or unsubstituted carbocyclyl, substituted or unsubstituted carbocyclylalkyl, substituted or unsubstituted aryl, substituted or unsubstituted aralkyl, substituted or unsubstituted heterocycloalkyl, substituted or unsubstituted heterocycloalkylalkyl, substituted or unsubstituted heteroaryl, or substituted or unsubstituted heteroarylalkyl; t is 1 or 2; and wherein any of R⁷, R⁸, R⁹, R¹⁰, R¹¹, R¹², R^{a}, and R^{b} can optionally be linked together.

In some embodiments, the disclosure provides compounds of Formula I, or any one of Formula 11, Formula 12, Formula 13, Formula 14, or Formula 15, wherein: R⁷, R⁸, R⁹, R¹⁰, R¹¹, R¹², R^{a}, and R^{b} are independently selected from hydrogen, and substituted or unsubstituted alkyl. In some embodiments, at least one of R^{c} and R^{d} is hydrogen. In some embodiments, both R^{c} and R^{d} are hydrogen.

In some embodiments, the disclosure provides a compound of any one of the following Formulas wherein D is defined as described herein:

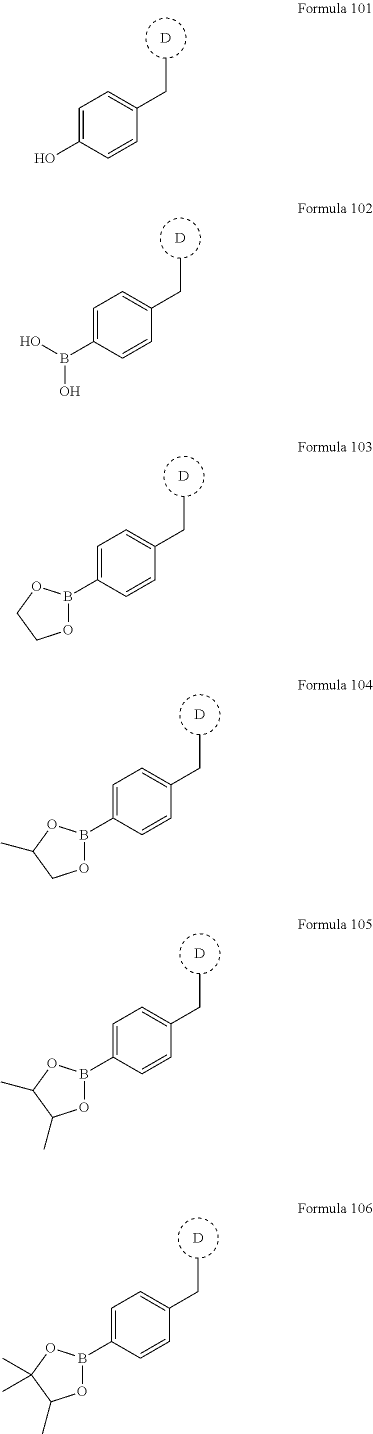

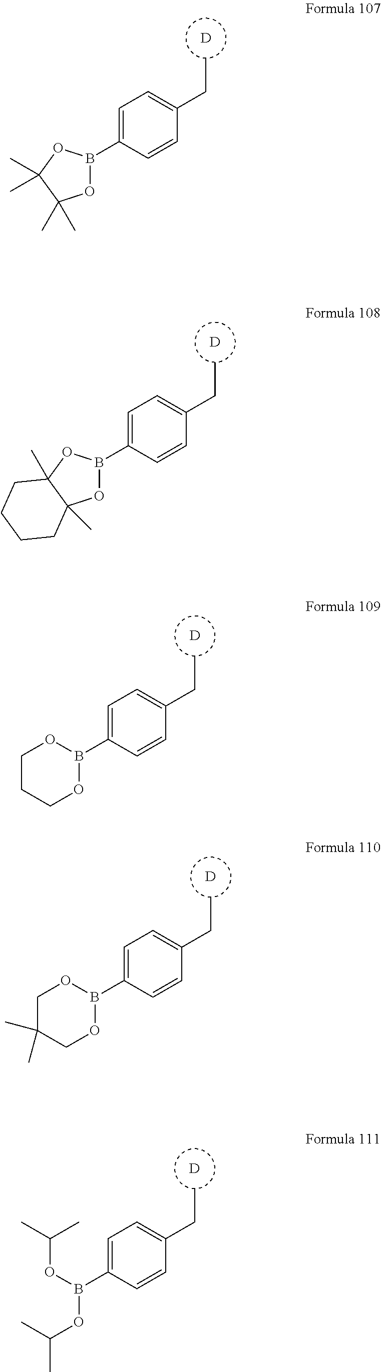

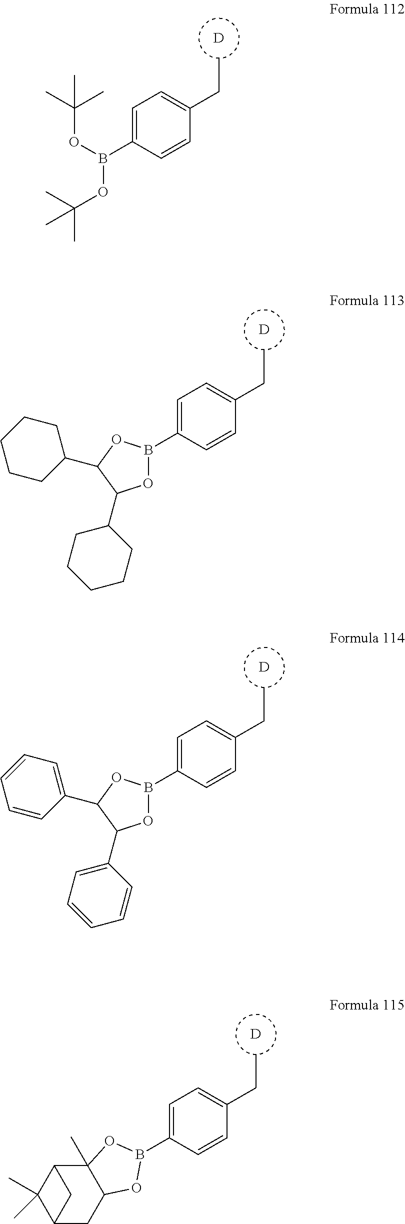

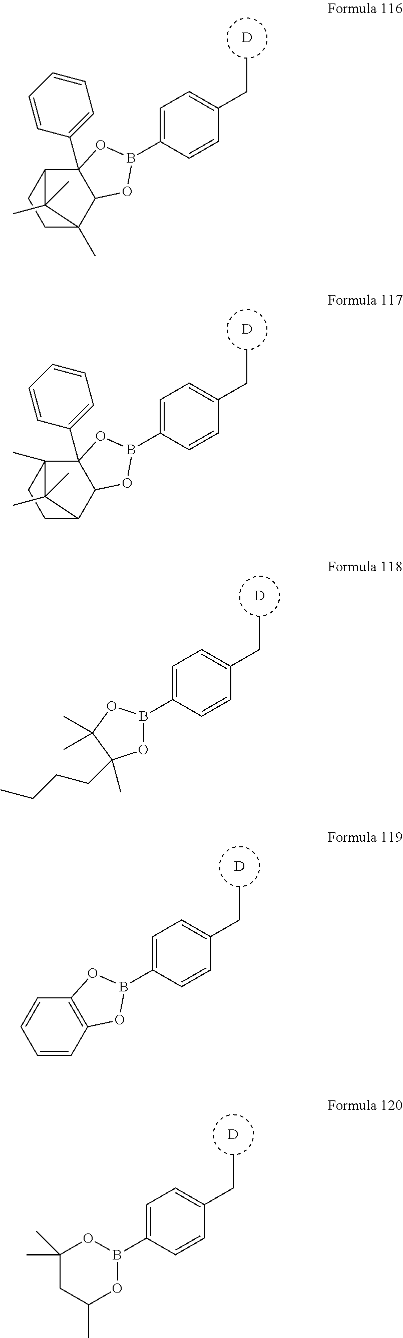

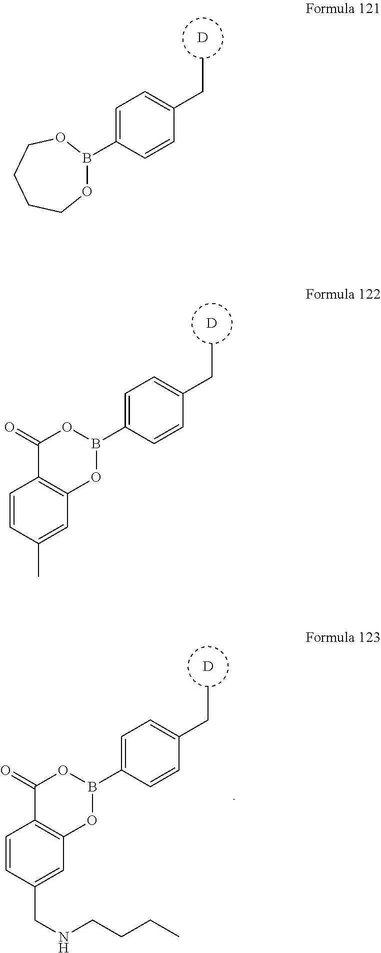

In some embodiments, the disclosure provides compounds of Formula I, or any one of Formula 11, Formula 12, Formula 13, Formula 14, or Formula 15, or any one of Formulas 101 to 123, wherein D comprises a pyrimidine moiety.

In some embodiments, the disclosure provides compounds of Formula I, or any one of Formula 11, Formula 12, Formula 13, Formula 14, or Formula 15, or any one of Formulas 101 to 123, wherein D is

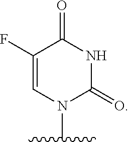 [SMILES: *n1cc(F)c(=O)[nH]c1=O]

In some embodiments, the disclosure provides compounds of Formula I, or any one of Formula 11, Formula 12, Formula 13, Formula 14, or Formula 15, or any one of Formulas 101 to 123, wherein D is

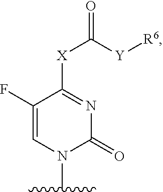

and wherein X and Y are independently selected from —O— and —NR^{a}—, and R⁶ is selected from hydrogen, substituted or unsubstituted alkyl, and substituted or unsubstituted heteroalkyl.

In some embodiments, the disclosure provides compounds of Formula I, or any one of Formula 11, Formula 12, Formula 13, Formula 14, or Formula 15, or any one of Formulas 101 to 123, wherein D comprises a purine moiety. In some embodiments, D is

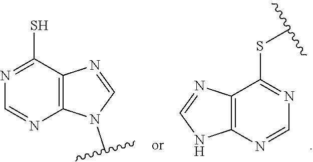 [SMILES: *n1cnc2c(S)ncnc21 ; *Sc1ncnc2[nH]cnc12]

In some embodiments, the disclosure provides compounds of Formula I, or any one of Formula 11, Formula 12, Formula 13, Formula 14, or Formula 15 or any one of Formulas 101 to 123, wherein D comprises a moiety selected from:

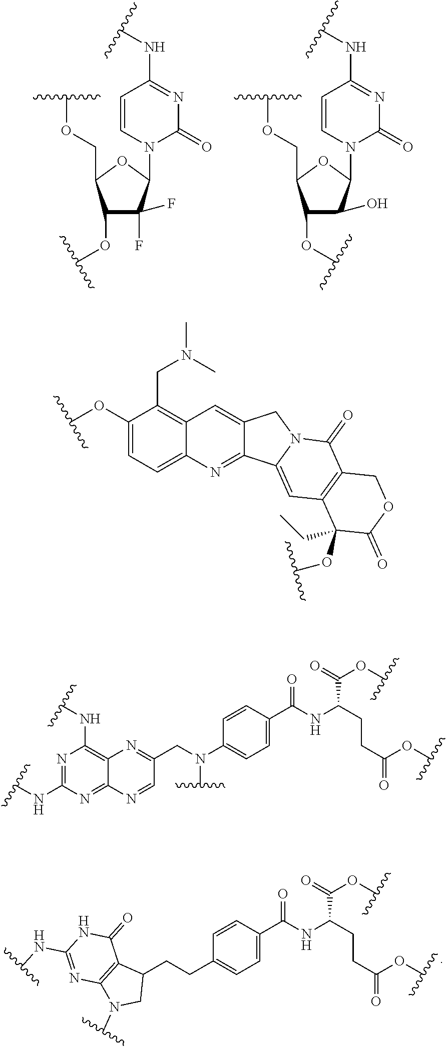

In some embodiments, the disclosure provides compounds of Formula I, or any one of Formula 11, Formula 12, Formula 13, Formula 14, or Formula 15, or any one of Formulas 101 to 123, wherein D is selected from:

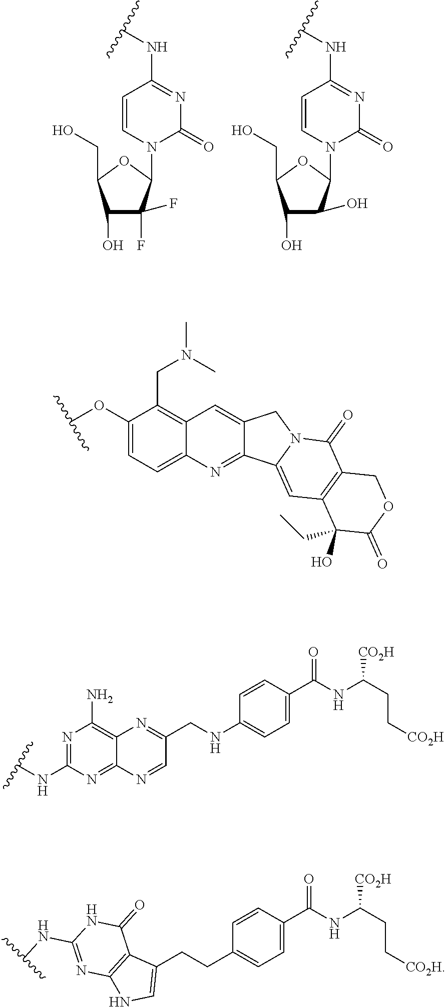

In some embodiments, the disclosure provides a compound of Formula A, Formula B, Formula C, or Formula D, or a pharmaceutically acceptable salt, solvate, hydrate, cocrystal, or prodrug thereof:

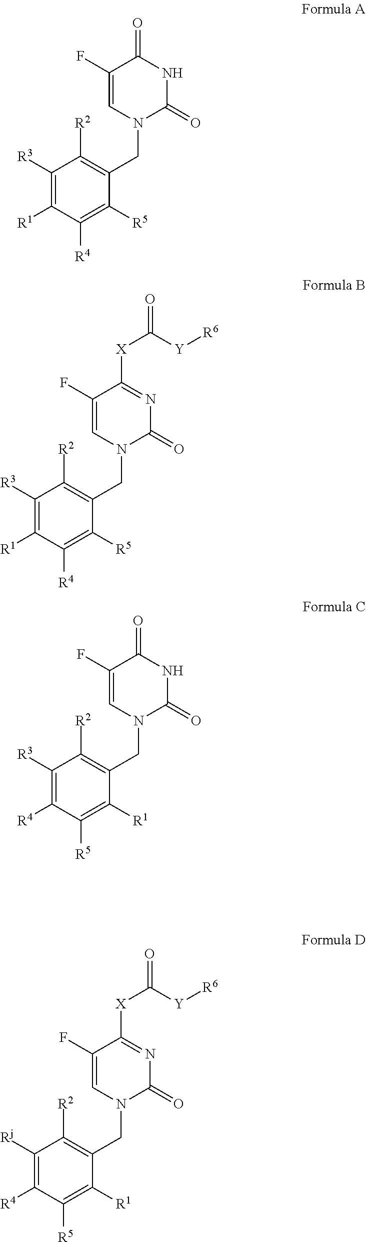

wherein in Formulas A-D: R¹ is —OR^{a} or —B(OR^{a})OR^{b}; X and Y are independently selected from —O— and —NR^{a}—; and R², R³, R⁴, R⁵, and R⁶ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted alkynyl, substituted or unsubstituted heteroalkynyl, substituted or unsubstituted cycloalkynyl, substituted or unsubstituted heterocycloalkynyl, substituted or unsubstituted aryl, substituted or unsubstituted heteroaryl, hydroxy, halo, cyano, trifluoromethyl, trifluoromethoxy, nitro, trimethylsilanyl, —OR^{a}, —SR^{a}, —OC(O)—R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{b}, —N(R^{a})C(O)R^{b}, —N(R^{a})C(O)N(R^{a})R^{b}, —N(R^{a})C(NR^{a})N(R^{a})R^{b}, —N(R^{a})S(O)ₜR^{b}, —S(O)ₜR^{a} (where t is 1 or 2), —S(O)ₜOR^{a}, —S(O)ₜN(R^{a})R^{b}, and —PO(OR^{a})(OR^{b}); R^{a} and R^{b} are independently selected from hydrogen, substituted or unsubstituted alkyl, substituted or unsubstituted fluoroalkyl, substituted or unsubstituted carbocyclyl, substituted or unsubstituted carbocyclylalkyl, substituted or unsubstituted aryl, substituted or unsubstituted aralkyl, substituted or unsubstituted heterocycloalkyl, substituted or unsubstituted heterocycloalkylalkyl, substituted or unsubstituted heteroaryl, or substituted or unsubstituted heteroarylalkyl; t is 1 or 2; and wherein any of R², R³, R⁴, R⁵, R⁶, R^{a}, and R^{b} can optionally be linked together.

In some embodiments, the disclosure provides a compound of any one of Formulas 16 to 25:

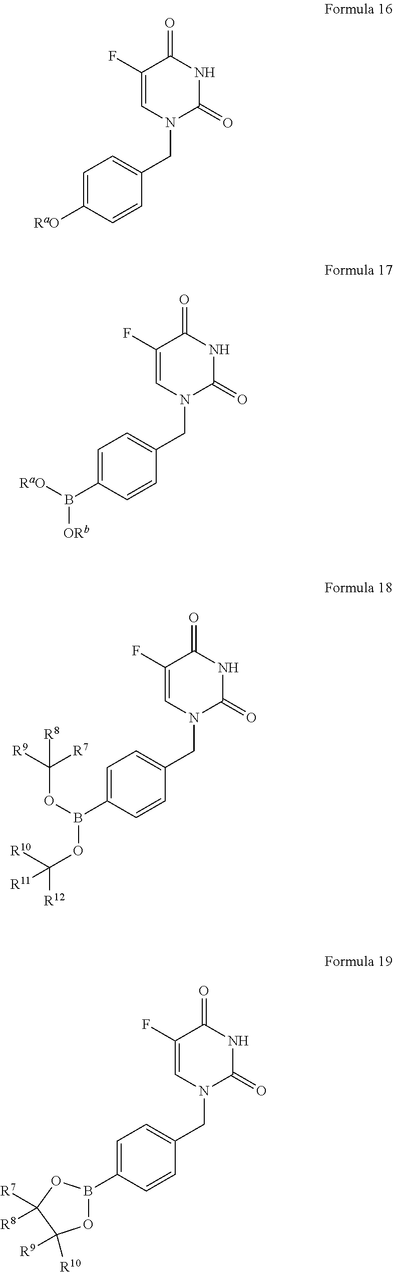

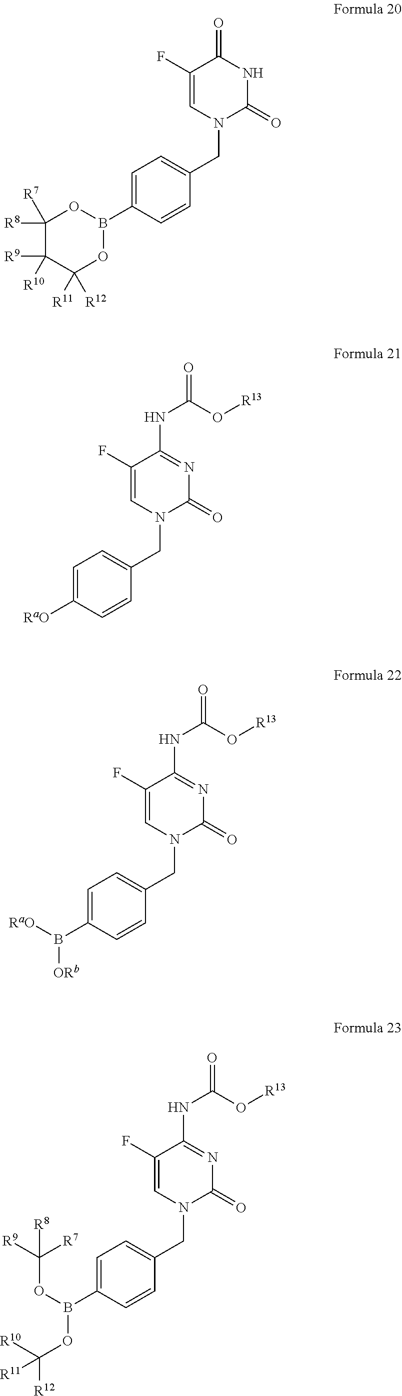

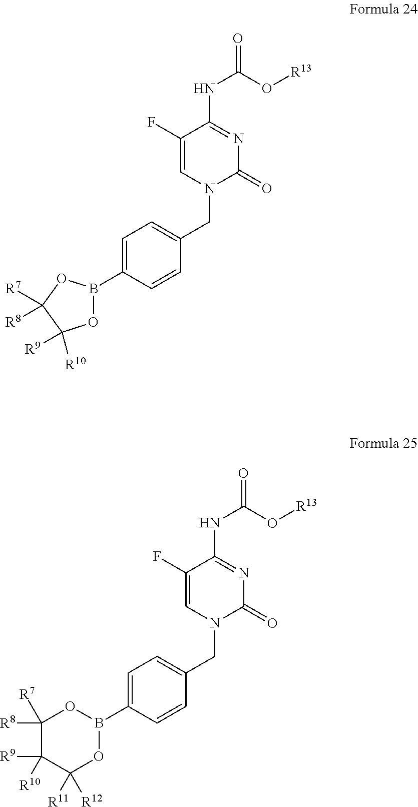

wherein in Formulas 16 to 25: R⁷, R¹, R⁹, R¹⁰, R¹¹, R¹², and R¹³ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted alkynyl, substituted or unsubstituted heteroalkynyl, substituted or unsubstituted cycloalkynyl, substituted or unsubstituted heterocycloalkynyl, substituted or unsubstituted aryl, substituted or unsubstituted heteroaryl, hydroxy, halo, cyano, trifluoromethyl, trifluoromethoxy, nitro, trimethylsilanyl, —OR^{a}, —SR^{a}, —OC(O)—R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{b}, —N(R^{a})C(O)R^{b}, —N(R^{a})C(O)N(R^{a})R^{b}, —N(R^{a})C(NR^{a})N(R^{a})R^{b}, —N(R^{a})S(O)ₜR^{b}, —S(O)ₜR^{a} (where t is 1 or 2), —S(O)ₜOR^{a}, —S(O)ₜN(R^{a})R^{b}, and —PO(OR^{a})(OR^{b}); R^{a} and R^{b} are independently selected from hydrogen, substituted or unsubstituted alkyl, substituted or unsubstituted fluoroalkyl, substituted or unsubstituted carbocyclyl, substituted or unsubstituted carbocyclylalkyl, substituted or unsubstituted aryl, substituted or unsubstituted aralkyl, substituted or unsubstituted heterocycloalkyl, substituted or unsubstituted heterocycloalkylalkyl, substituted or unsubstituted heteroaryl, or substituted or unsubstituted heteroarylalkyl; t is 1 or 2; and wherein any of R⁷, R⁸, R⁹, R¹⁰, R¹¹, R¹², R^{a}, and R^{b} can optionally be linked together. In some embodiments, the disclosure provides a compound of any one of Formulas 16 to 25, wherein: R⁷, R⁸, R⁹, R¹⁰, R¹¹, R¹², R¹³, R^{a}, and R^{b} are independently selected from hydrogen, and substituted or unsubstituted alkyl.

In some embodiments, the disclosure provides compounds of any one of Formulas 1001 to 1023:

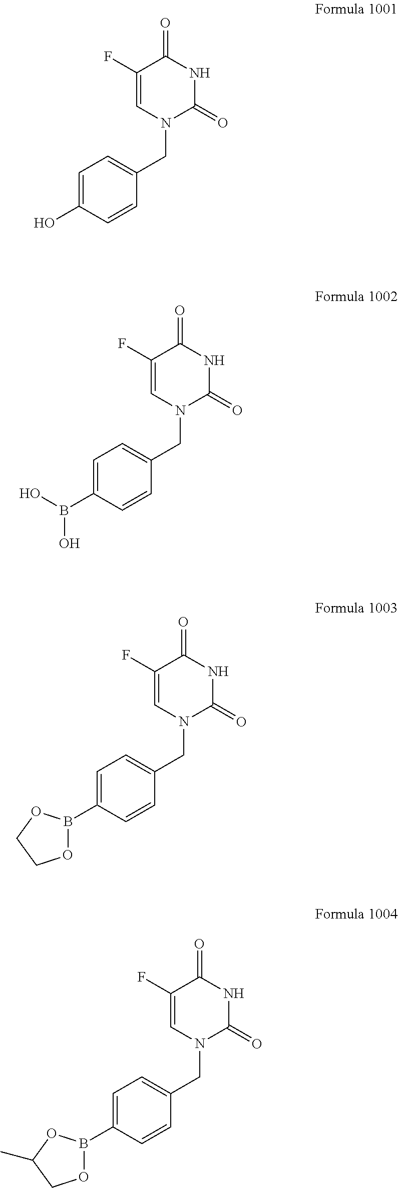 [SMILES: O=c1[nH]c(=O)n(Cc2ccc(O)cc2)cc1F ; O=c1[nH]c(=O)n(Cc2ccc(B(O)O)cc2)cc1F ; O=c1[nH]c(=O)n(Cc2ccc(B3OCCO3)cc2)cc1F ; CC1COB(c2ccc(Cn3cc(F)c(=O)[nH]c3=O)cc2)O1]

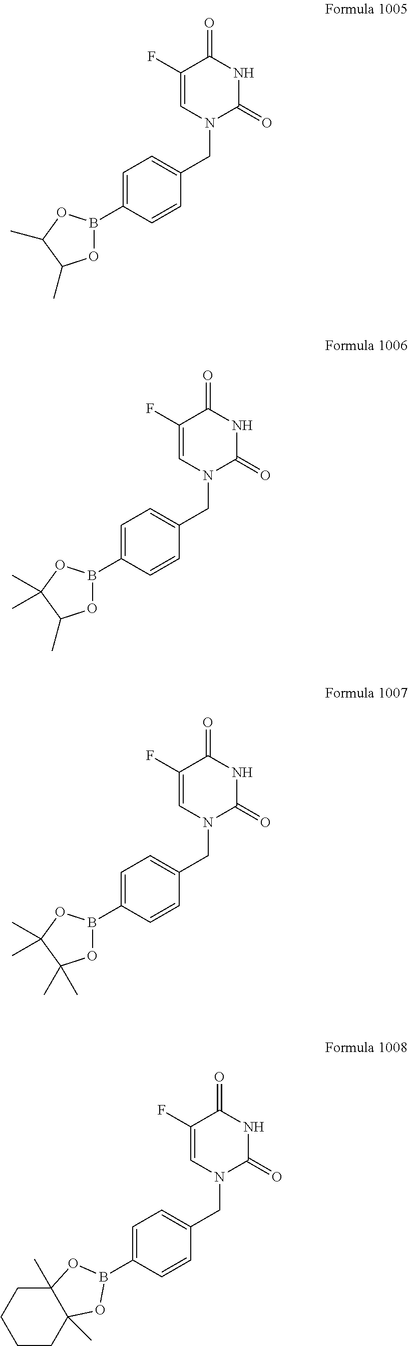 [SMILES: CC1OB(c2ccc(Cn3cc(F)c(=O)[nH]c3=O)cc2)OC1C ; CC1OB(c2ccc(Cn3cc(F)c(=O)[nH]c3=O)cc2)OC1(C)C ; CC1(C)OB(c2ccc(Cn3cc(F)c(=O)[nH]c3=O)cc2)OC1(C)C ; CC12CCCCC1(C)OB(c1ccc(Cn3cc(F)c(=O)[nH]c3=O)cc1)O2]

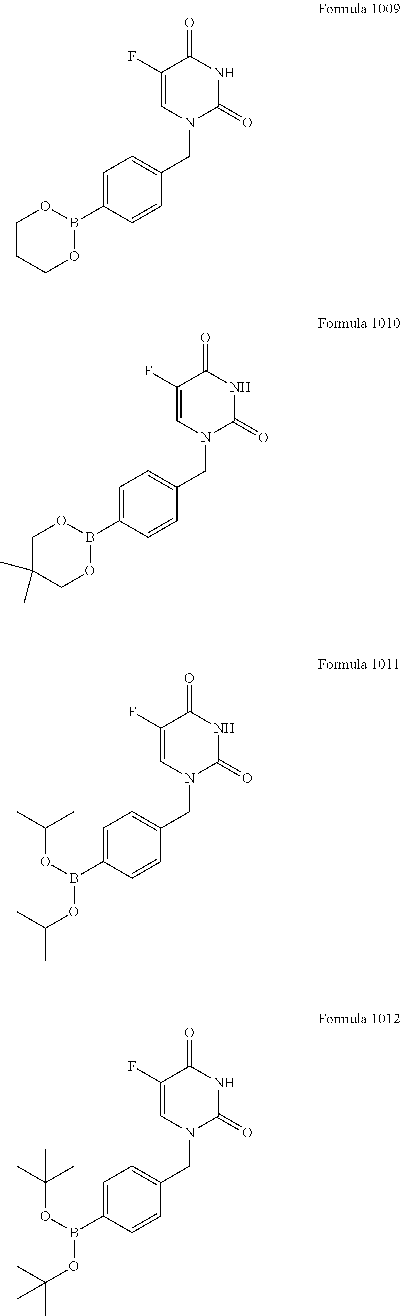 [SMILES: O=c1[nH]c(=O)n(Cc2ccc(B3OCCCO3)cc2)cc1F ; CC1(C)COB(c2ccc(Cn3cc(F)c(=O)[nH]c3=O)cc2)OC1 ; CC(C)OB(OC(C)C)c1ccc(Cn2cc(F)c(=O)[nH]c2=O)cc1 ; CC(C)(C)OB(OC(C)(C)C)c1ccc(Cn2cc(F)c(=O)[nH]c2=O)cc1]

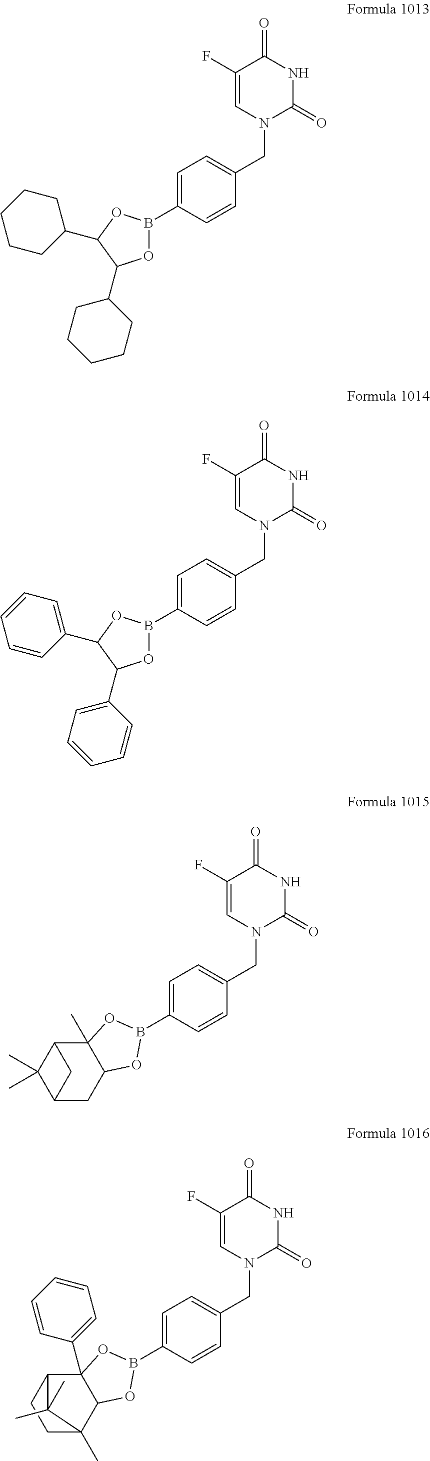 [SMILES: O=c1[nH]c(=O)n(Cc2ccc(B3OC(C4CCCCC4)C(C4CCCCC4)O3)cc2)cc1F ; O=c1[nH]c(=O)n(Cc2ccc(B3OC(c4ccccc4)C(c4ccccc4)O3)cc2)cc1F ; CC1(C)C2CC3OB(c4ccc(Cn5cc(F)c(=O)[nH]c5=O)cc4)OC3(C)C1C2 ; CC1(C)C2CCC1(C)C1OB(c3ccc(Cn4cc(F)c(=O)[nH]c4=O)cc3)OC21c1ccccc1]

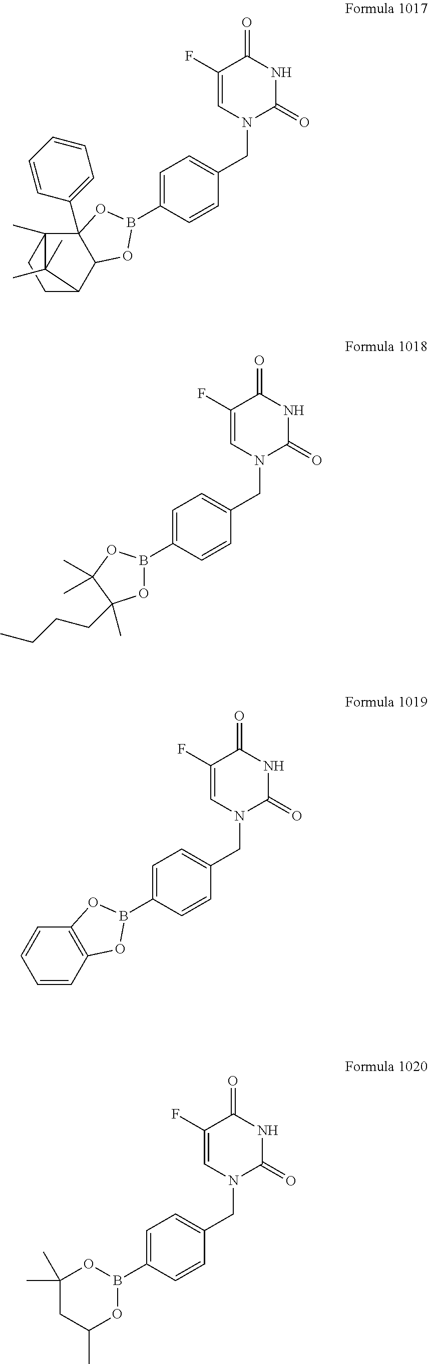 [SMILES: CC1(C)C2CCC1(C)C1(c3ccccc3)OB(c3ccc(Cn4cc(F)c(=O)[nH]c4=O)cc3)OC21 ; CCCCC1(C)OB(c2ccc(Cn3cc(F)c(=O)[nH]c3=O)cc2)OC1(C)C ; O=c1[nH]c(=O)n(Cc2ccc(B3Oc4ccccc4O3)cc2)cc1F ; CC1CC(C)(C)OB(c2ccc(Cn3cc(F)c(=O)[nH]c3=O)cc2)O1]

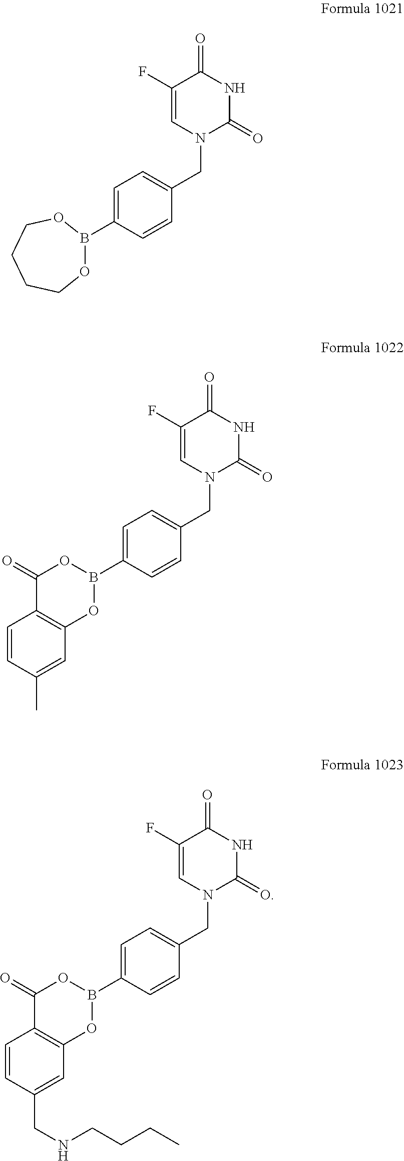 [SMILES: O=c1[nH]c(=O)n(Cc2ccc(B3OCCCCO3)cc2)cc1F ; Cc1ccc2c(c1)OB(c1ccc(Cn3cc(F)c(=O)[nH]c3=O)cc1)OC2=O ; CCCCNCc1ccc2c(c1)OB(c1ccc(Cn3cc(F)c(=O)[nH]c3=O)cc1)OC2=O]

In some embodiments, the disclosure provides compounds of Formula 2001, Formula 2002, or Formula 2003:

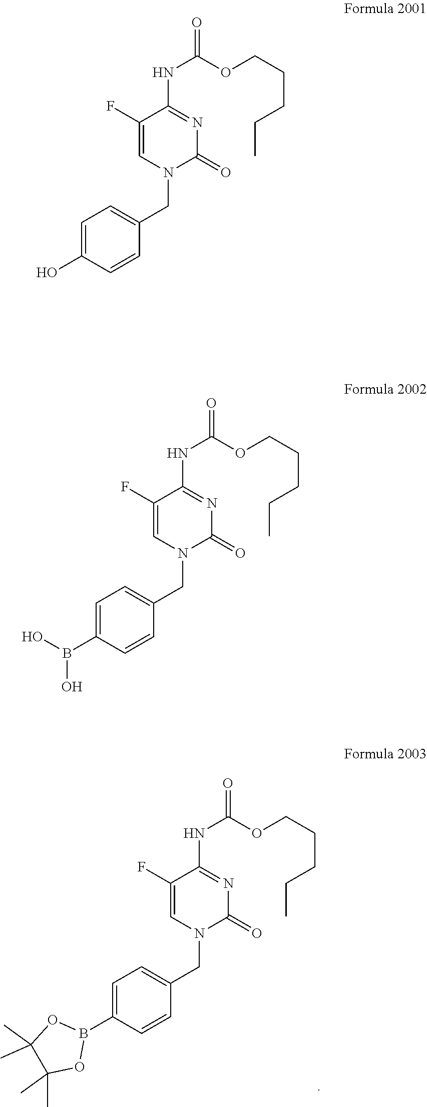 [SMILES: CCCCCOC(=O)Nc1nc(=O)n(Cc2ccc(O)cc2)cc1F ; CCCCCOC(=O)Nc1nc(=O)n(Cc2ccc(B(O)O)cc2)cc1F ; CCCCCOC(=O)Nc1nc(=O)n(Cc2ccc(B3OC(C)(C)C(C)(C)O3)cc2)cc1F]

In some embodiments, the disclosure provides compounds any one of Formulas 1001-a to 1023-a:

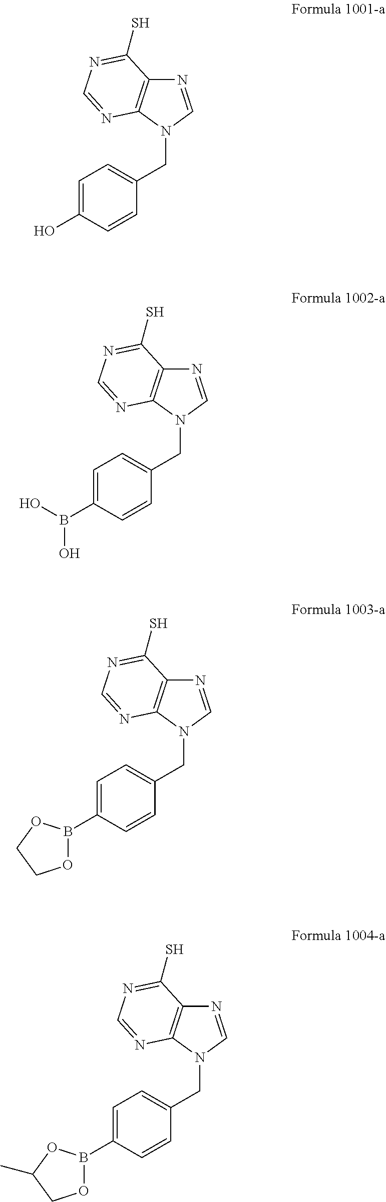 [SMILES: Oc1ccc(Cn2cnc3c(S)ncnc32)cc1 ; OB(O)c1ccc(Cn2cnc3c(S)ncnc32)cc1 ; Sc1ncnc2c1ncn2Cc1ccc(B2OCCO2)cc1 ; CC1COB(c2ccc(Cn3cnc4c(S)ncnc43)cc2)O1]

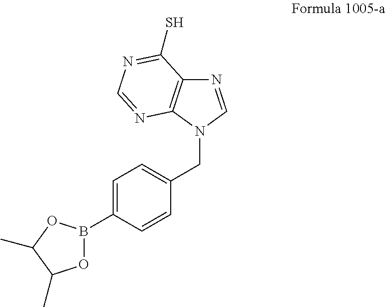 [SMILES: CC1OB(c2ccc(Cn3cnc4c(S)ncnc43)cc2)OC1C]

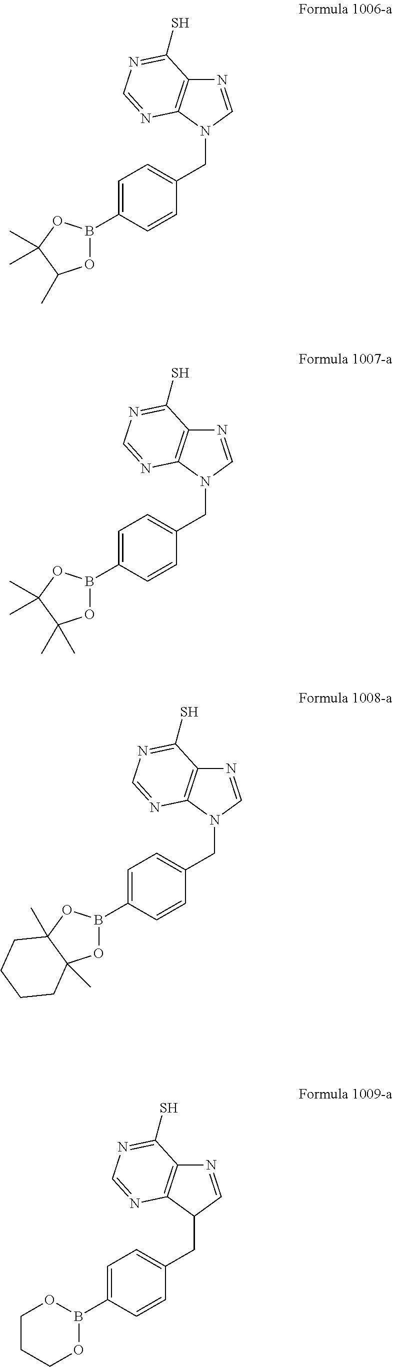 [SMILES: CC1OB(c2ccc(Cn3cnc4c(S)ncnc43)cc2)OC1(C)C ; CC1(C)OB(c2ccc(Cn3cnc4c(S)ncnc43)cc2)OC1(C)C ; CC12CCCCC1(C)OB(c1ccc(Cn3cnc4c(S)ncnc43)cc1)O2 ; Sc1ncnc2c1N=CC2Cc1ccc(B2OCCCO2)cc1]

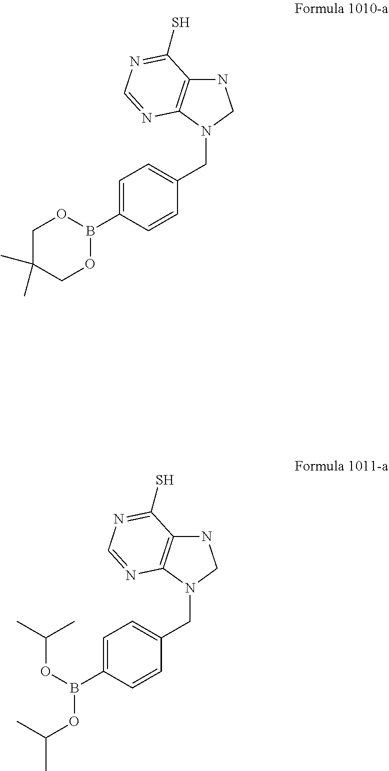 [SMILES: CC1(C)COB(c2ccc(CN3CNc4c(S)ncnc43)cc2)OC1 ; CC(C)OB(OC(C)C)c1ccc(CN2CNc3c(S)ncnc32)cc1]

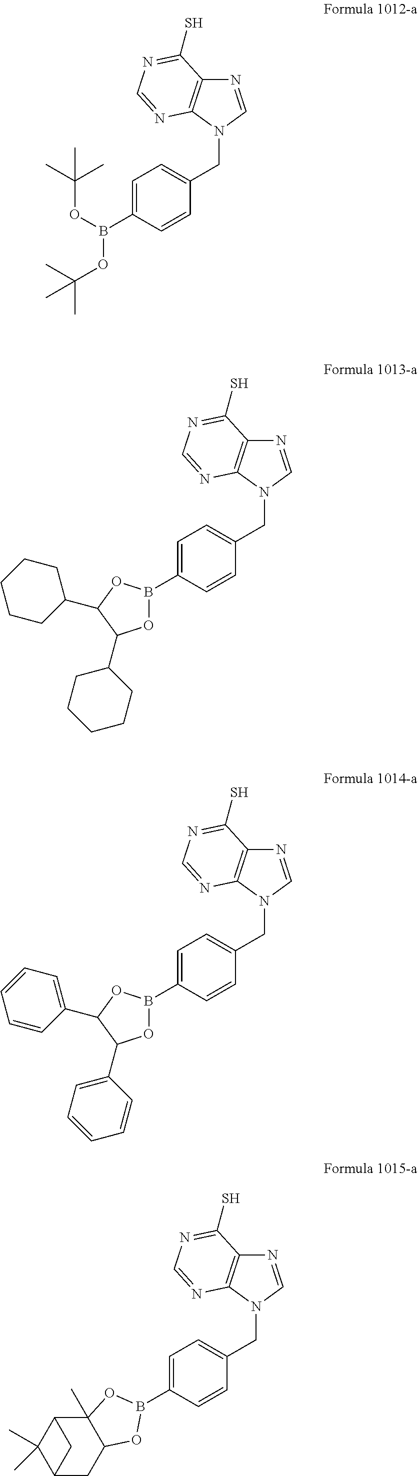 [SMILES: CC(C)(C)OB(OC(C)(C)C)c1ccc(Cn2cnc3c(S)ncnc32)cc1 ; Sc1ncnc2c1ncn2Cc1ccc(B2OC(C3CCCCC3)C(C3CCCCC3)O2)cc1 ; Sc1ncnc2c1ncn2Cc1ccc(B2OC(c3ccccc3)C(c3ccccc3)O2)cc1 ; CC1(C)C2CC3OB(c4ccc(Cn5cnc6c(S)ncnc65)cc4)OC3(C)C1C2]

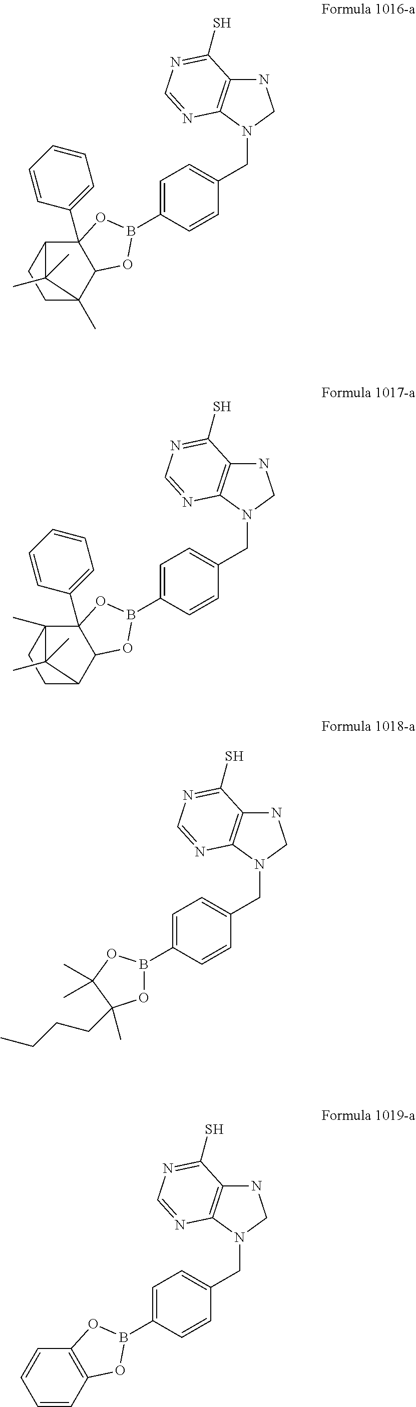 [SMILES: CC1(C)C2CCC1(C)C1OB(c3ccc(CN4CNc5c(S)ncnc54)cc3)OC21c1ccccc1 ; CC1(C)C2CCC1(C)C1(c3ccccc3)OB(c3ccc(CN4CNc5c(S)ncnc54)cc3)OC21 ; CCCCC1(C)OB(c2ccc(CN3CNc4c(S)ncnc43)cc2)OC1(C)C ; Sc1ncnc2c1NCN2Cc1ccc(B2Oc3ccccc3O2)cc1]

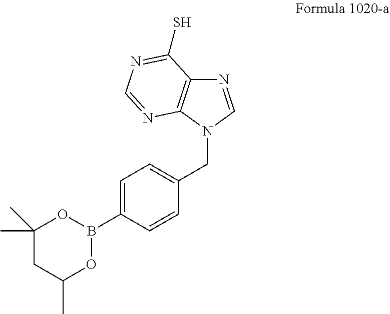 [SMILES: CC1CC(C)(C)OB(c2ccc(Cn3cnc4c(S)ncnc43)cc2)O1]

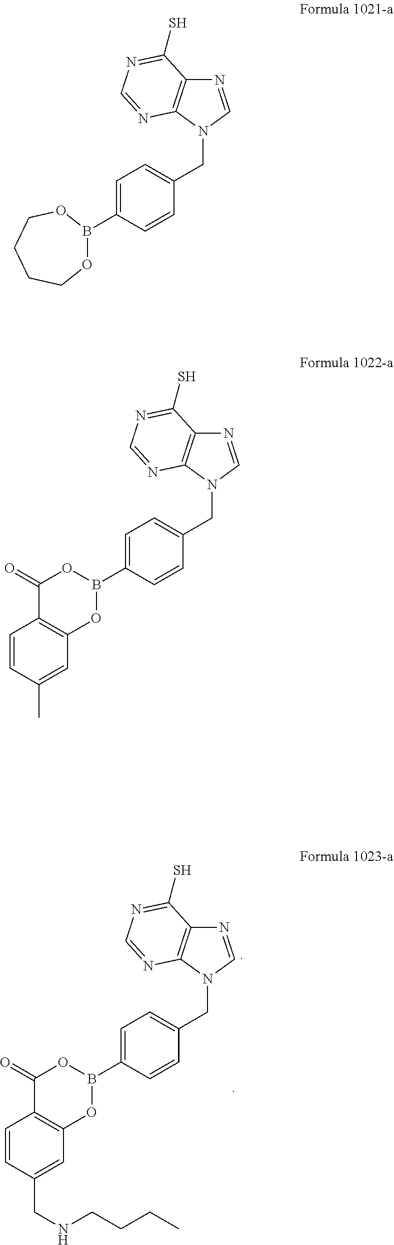 [SMILES: Sc1ncnc2c1ncn2Cc1ccc(B2OCCCCO2)cc1 ; Cc1ccc2c(c1)OB(c1ccc(Cn3cnc4c(S)ncnc43)cc1)OC2=O ; CCCCNCc1ccc2c(c1)OB(c1ccc(Cn3cnc4c(S)ncnc43)cc1)OC2=O]

In some embodiments, the disclosure provides compounds of any one of Formulas 1001-b to 1023-b:

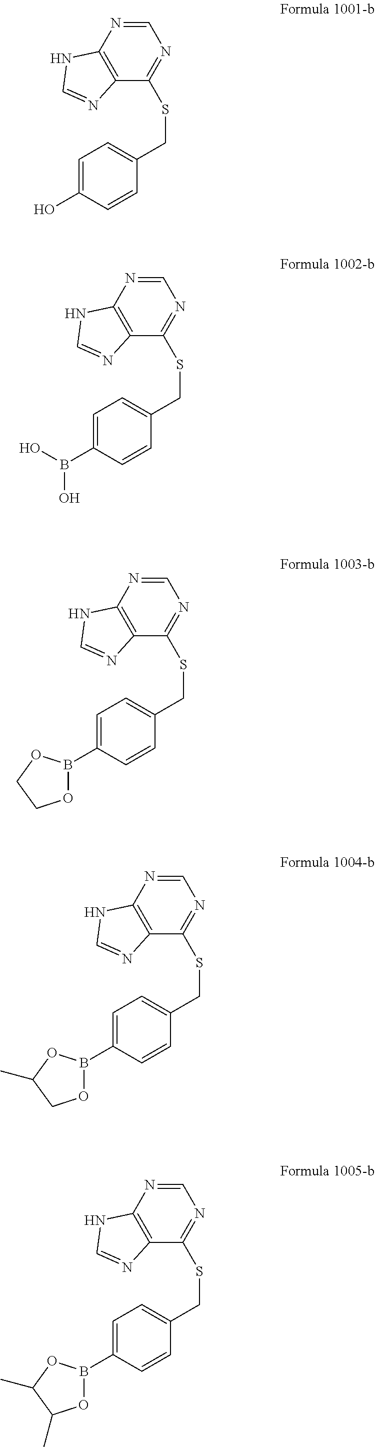 [SMILES: Oc1ccc(CSc2ncnc3[nH]cnc23)cc1 ; OB(O)c1ccc(CSc2ncnc3[nH]cnc23)cc1 ; c1nc(SCc2ccc(B3OCCO3)cc2)c2nc[nH]c2n1 ; CC1COB(c2ccc(CSc3ncnc4[nH]cnc34)cc2)O1 ; CC1OB(c2ccc(CSc3ncnc4[nH]cnc34)cc2)OC1C]

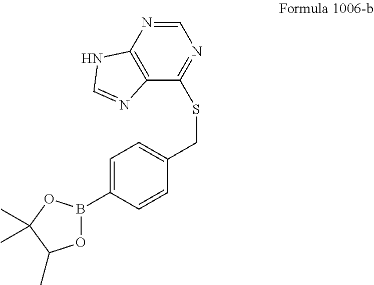 [SMILES: CC1OB(c2ccc(CSc3ncnc4[nH]cnc34)cc2)OC1(C)C]

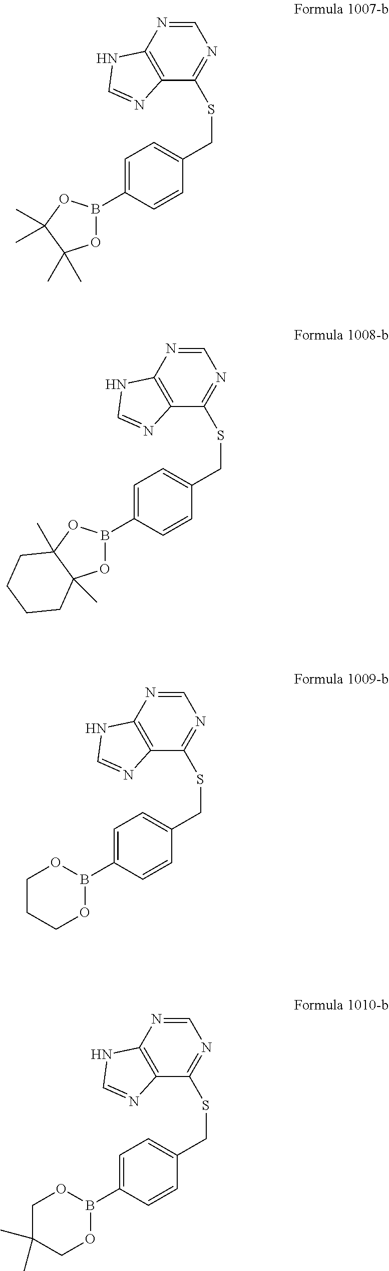 [SMILES: CC1(C)OB(c2ccc(CSc3ncnc4[nH]cnc34)cc2)OC1(C)C ; CC12CCCCC1(C)OB(c1ccc(CSc3ncnc4[nH]cnc34)cc1)O2 ; c1nc(SCc2ccc(B3OCCCO3)cc2)c2nc[nH]c2n1 ; CC1(C)COB(c2ccc(CSc3ncnc4[nH]cnc34)cc2)OC1]

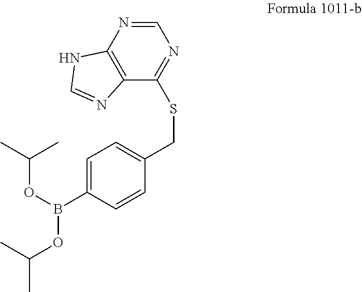 [SMILES: CC(C)OB(OC(C)C)c1ccc(CSc2ncnc3[nH]cnc23)cc1]

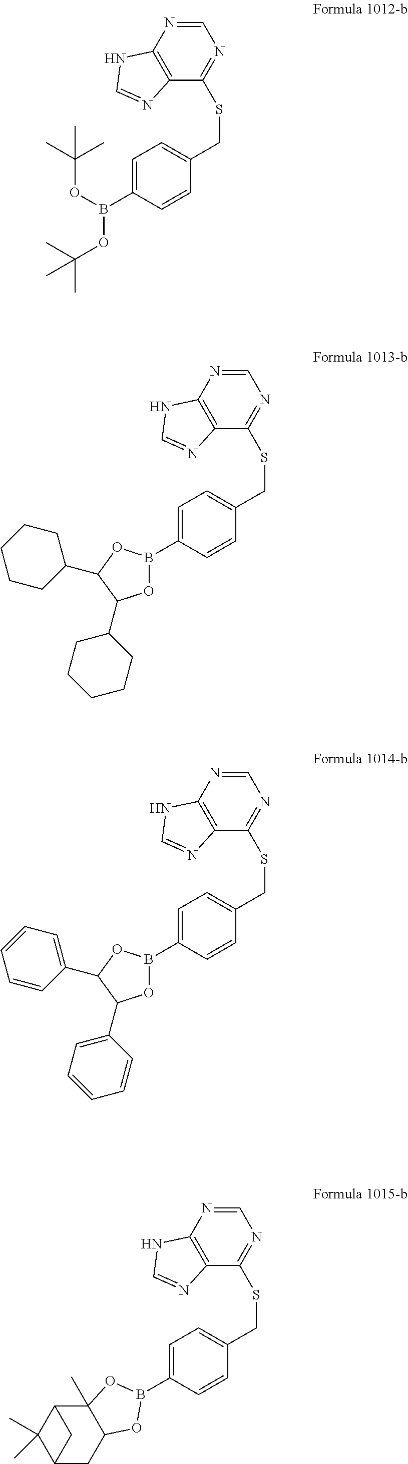 [SMILES: CC(C)(C)OB(OC(C)(C)C)c1ccc(CSc2ncnc3[nH]cnc23)cc1 ; c1nc(SCc2ccc(B3OC(C4CCCCC4)C(C4CCCCC4)O3)cc2)c2nc[nH]c2n1 ; c1ccc(C2OB(c3ccc(CSc4ncnc5[nH]cnc45)cc3)OC2c2ccccc2)cc1 ; CC1(C)C2CC3OB(c4ccc(CSc5ncnc6[nH]cnc56)cc4)OC3(C)C1C2]

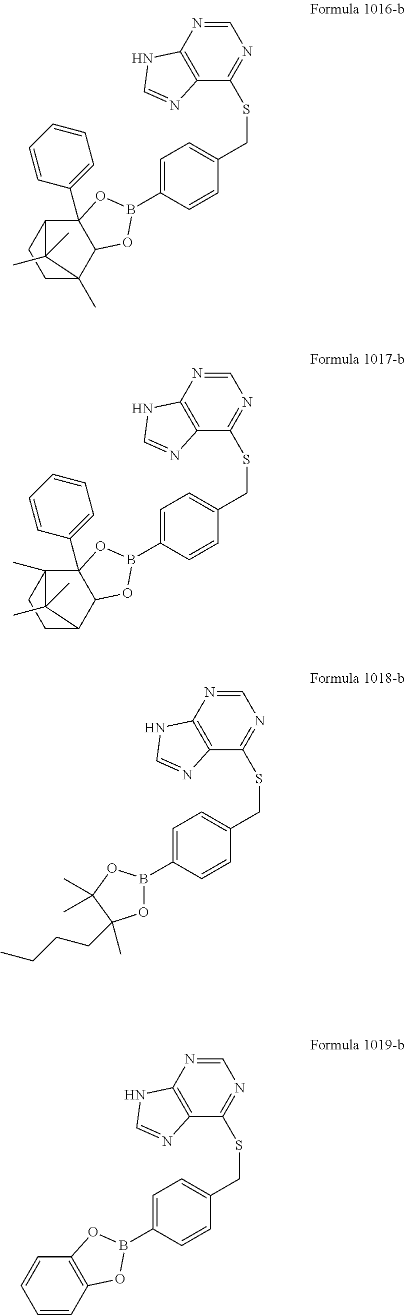 [SMILES: CC1(C)C2CCC1(C)C1OB(c3ccc(CSc4ncnc5[nH]cnc45)cc3)OC21c1ccccc1 ; CC1(C)C2CCC1(C)C1(c3ccccc3)OB(c3ccc(CSc4ncnc5[nH]cnc45)cc3)OC21 ; CCCCC1(C)OB(c2ccc(CSc3ncnc4[nH]cnc34)cc2)OC1(C)C ; c1ccc2c(c1)OB(c1ccc(CSc3ncnc4[nH]cnc34)cc1)O2]

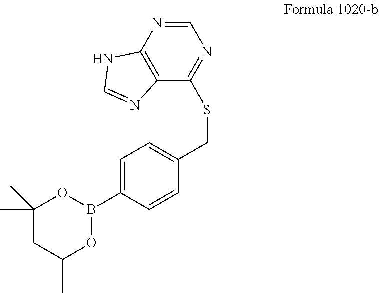 [SMILES: CC1CC(C)(C)OB(c2ccc(CSc3ncnc4[nH]cnc34)cc2)O1]

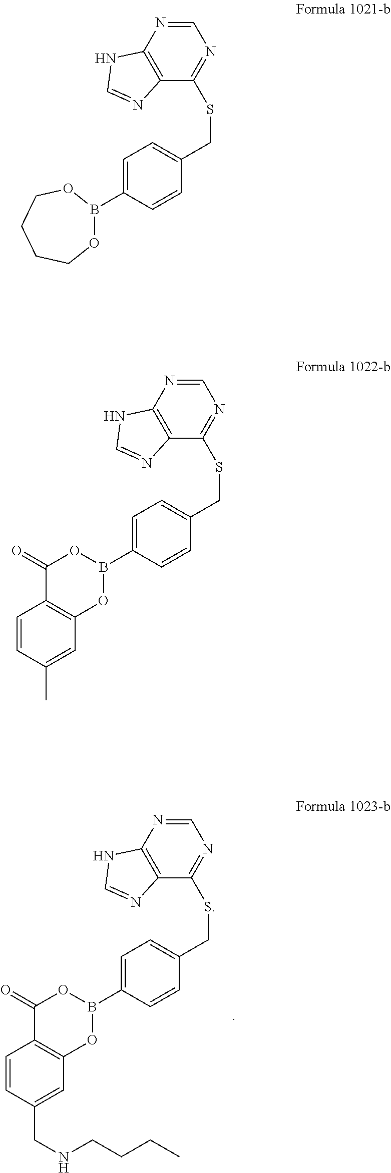 [SMILES: c1nc(SCc2ccc(B3OCCCCO3)cc2)c2nc[nH]c2n1 ; Cc1ccc2c(c1)OB(c1ccc(CSc3ncnc4[nH]cnc34)cc1)OC2=O ; CCCCNCc1ccc2c(c1)OB(c1ccc(CSc3ncnc4[nH]cnc34)cc1)OC2=O]

The disclosure also provides a pharmaceutical composition comprising a compound of Formula I, or any one of Formulas A to D, or any one of Formulas 111 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, or a pharmaceutically acceptable salt, solvate, hydrate, cocrystal, or prodrug thereof, and a pharmaceutically acceptable carrier.

The disclosure also provides a method of treating a disease alleviated by anti-cancer or anti-autoimmune diseases chemotherapy in a patient in need thereof, comprising administering to the patient a therapeutically effective amount of a compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, or a pharmaceutically acceptable salt, solvate, hydrate, cocrystal, or prodrug thereof. In some embodiments, the diseases is a disease alleviated by 5-fluorouracil (5-FU) chemotherapy. In some embodiments, the diseases is a disease alleviated by 6-mercaptopurine (6-MP) chemotherapy. In some embodiments, any side effects of the treatment are reduced compared to the side effects of a treatment including administering to the patient a therapeutically equivalent amount of 5-fluorouracil (5-FU) or 6-mercaptopurine (6-MP).

The disclosure also provides a method of treating a disease alleviated by anti-cancer or anti-autoimmune diseases chemotherapy in a patient in need thereof, comprising administering to the patient a pharmaceutical composition comprising a compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, or a pharmaceutically acceptable salt, solvate, hydrate, cocrystal, or prodrug thereof, and a pharmaceutically acceptable carrier. In some embodiments, the diseases is a disease alleviated by 5-fluorouracil (5-FU) chemotherapy. In some embodiments, the diseases is a disease alleviated by 6-mercaptopurine (6-MP) chemotherapy.

In some embodiments, the disclosure provides methods of treating a disease alleviated by anti-cancer or anti-autoimmune diseases chemotherapy in a patient in need thereof, comprising administering to the patient a compound or a pharmaceutical composition thereof described herein, wherein the disease is cancer. In some embodiments, the cancer is a solid tumor cancer. In some embodiments, the cancer is a hematological malignancy. In some embodiments, the cancer is selected from malignant pancreatic insulinoma, malignant carcinoid carcinoma, mycosis fungoides, malignant hypercalcemia, cervical hyperplasia, leukemia, myeloid leukemia, acute myeloid leukemia, acute lymphocytic leukemia, chronic lymphocytic leukemia, acute myelogenous leukemia, chronic myelogenous leukemia, chronic granulocytic leukemia, chronic myeloid leukemia, acute granulocytic leukemia, hairy cell leukemia, acute erythroleukemic leukemia, acute lymphoblastic B-cell leukemia, acute lymphoblastic T-cell leukemia, acute lymphoblastic leukemia, acute megakaryoblastic leukemia, acute monoblastic leukemia, acute myeloblastic leukemia, acute myelomonocytic leukemia, acute nonlymphocytic leukemia, acute promyelocytic leukemia, acute undifferentiated leukemia, chronic lymphocytic leukemia, chronic myelocytic leukemia, hairy cell leukemia, rhabdomyosarcoma, Kaposi's sarcoma, essential thrombocytosis, soft-tissue sarcoma, retinoblastoma, acoustic neuroma, adenocarcinoma, angiosarcoma, adrenal carcinoma, adrenal cortex carcinoma, astrocytoma, basal cell carcinoma, bile duct carcinoma, bladder cancer, bladder carcinoma, brain cancer, breast cancer, breast carcinoma, bronchogenic carcinoma, cervical cancer, cervical carcinoma, chordoma, choriocarcinoma, colon cancer, colon carcinoma, colorectal cancer, craniopharyngioma, cystadenocarcinoma, embryonal carcinoma, endotheliocarcinoma, endometrial carcinoma, ependymoma, epithelial carcinoma, esophageal cancer, esophageal carcinoma, Ewing's tumor, fibrosarcoma, gastric cancer, genitourinary carcinoma, glioblastoma, glioblastoma multiforme, glioma, head and neck cancer, hemangioblastoma, hepatoma, kidney cancer, leiomyosarcoma, liposarcoma, liver cancer, lung cancer, lung carcinoma, lymphangioendotheliosarcoma, lymphangiosarcoma, lymphoma, medullary carcinoma, medulloblastoma, malignant melanoma, melanoma, meningioma, mesothelioma, myxosarcoma, myeloma, nasal cancer, neuroblastoma, non-small cell lung cancer, oligodendroglioma, oral cancer, osteogenic sarcoma, ovarian cancer, ovarian carcinoma, pancreatic cancer, pancreatic carcinoma, papillary adenocarcinoma, papillary carcinoma, pinealoma, prostate cancer, prostatic carcinoma, primary brain carcinoma, rabdomyosarcoma, rectal cancer, renal cell carcinoma, retinoblastoma, sarcoma, sebaceous gland carcinoma, seminoma, skin cancer, squamous cell carcinoma, stomach cancer, stomach carcinoma, sweat gland carcinoma, synovioma, testicular cancer, testicular carcinoma, thyroid carcinoma, small cell lung carcinoma, throat cancer, uterine cancer, Wilm's tumor, blood cancer, heavy chain disease, Hodgkin's disease, multiple myeloma, non-Hodgkin's lymphoma, polycythemia vera, primary macroglobulinemia, and Waldenstrom's macroglobulinemia. In some embodiments, the cancer is selected from leukemia, non-small-cell lung cancer, colon cancer, CNS cancer, melanoma, ovarian cancer, renal cancer, prostate cancer, and breast cancer. In some embodiments, the diseases is a disease alleviated by 5-fluorouracil (5-FU) chemotherapy. In some embodiments, the diseases is a disease alleviated by 6-mercaptopurine (6-MP) chemotherapy. In some embodiments, any side effects of the treatment are reduced compared to the side effects of a treatment including administering to the patient a therapeutically equivalent amount of 5-fluorouracil (5-FU) or 6-mercaptopurine (6-MP).

In some embodiments, the disclosure provides methods of treating a disease alleviated by anti-cancer or anti-autoimmune diseases chemotherapy in a patient in need thereof, comprising administering to the patient a compound or a pharmaceutical composition thereof described herein, wherein the disease is an autoimmune disease. In some embodiments, the autoimmune disease is selected from achalasia, Addison's disease, adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticarial, axonal and neuronal neuropathy (AMAN), Baló disease, Behcet's disease, benign mucosal pemphigoid, bullous pemphigoid, Castleman disease (CD), celiac disease, Chagas disease, chronic inflammatory demyelinating polyneuropathy (CIDP), chronic recurrent multifocal osteomyelitis (CRMO), Churg-Strauss syndrome (CSS), eosinophilic granulomatosis (EGPA), cicatricial pemphigoid, Cogan's syndrome, cold agglutinin disease, congenital heart block, Coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), herpes gestationis, pemphigoid gestationis (PG), hidradenitis suppurativa (HS; acne inversa), hypogammalglobulinemia, IgA nephropathy, IgG4-related sclerosing disease, immune thrombocytopenic purpura (ITP), inclusion body myositis (IBM), interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, Lyme disease chronic, Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, Pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes type I, II, III, polymyalgia rheumatic, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm and testicular autoimmunity, stiff person syndrome (SPS), subacute bacterial endocarditis (SBE), Susac's syndrome, sympathetic ophthalmia (SO), Takayasu's arteritis, temporal arteritis, giant cell arteritis, thrombocytopenic purpura (TTP), Tolosa-Hunt syndrome (THS), transverse myelitis, type 1 diabetes, ulcerative colitis (UC), undifferentiated connective tissue disease (UCTD), uveitis, vasculitis, vitiligo, and Vogt-Koyanagi-Harada Disease. In some embodiments, the diseases is a disease alleviated by 5-fluorouracil (5-FU) chemotherapy. In some embodiments, the diseases is a disease alleviated by 6-mercaptopurine (6-MP) chemotherapy. In some embodiments, any side effects of the treatment are reduced compared to the side effects of a treatment including administering to the patient a therapeutically equivalent amount of 5-fluorouracil (5-FU) or 6-mercaptopurine (6-MP).

### BRIEF DESCRIPTION OF THE DRAWINGS

The foregoing summary, as well as the following detailed description of the invention, will be better understood when read in conjunction with the appended drawings.

FIGS. 1A and 1B illustrate the release of 5-FU from compounds 1a-1b. Incubation of prodrugs 1a-1b (100 μM) was carried out in the presence of 5 equivalent of H₂O₂: (FIG. 1A) compound 1a and (FIG. 1B) compound 1b. The conversion of prodrugs 1a-1b and release of 5-FU was monitored. The data represent mean±SD of triplicates.

FIGS. 2A and 2B illustrate the anticancer effects and selectivity of 5-FU and compounds 1a-1c. Cell viability was analyzed after the breast cancer MCF7 cells (FIG. 2A) and normal MEC1 cells (FIG. 2B) were treated with 5-FU or compounds 1a-1c at different concentrations. Data are presented as mean±SD, n=4.

FIG. 3 illustrates the effect of ROS on the activation of 1a-b. Cell viability assay of MCF7 cancer cells treated with varying concentrations of 1a-b in the presence of 10 mM NAC. Pre-incubation with this ROS scavenger was carried out for 2 hours. Data are presented as mean SD, n=4.

FIGS. 4A and 4B illustrate the effect of compounds 1a-1b in apoptosis. FIG. 4A: immunofluorescence of DNA cleavage protein γH2Ax was enhanced by using 5-FU and prodrugs 1a-1b. FIG. 4B: immunoblot analysis of MCF-7 cells treated with 5-FU, prodrugs 1a-1b for apoptosis markers PARP1 and Caspase 3. The cells were treated for 48 h with the indicated concentrations of compounds.

FIGS. 5A and 5B illustrate the survival rate (FIG. 5A) and body weight (FIG. 5B) curves of the various groups of mice. Twenty-one mice were randomly distributed into three groups (seven mice in each group): vehicle (5% DMSO and 15% polyethylene glycol in saline), 100 mg/kg of 5-FU and 100 mg/kg of prodrug 1a. Mice were treated every other day by intraperitoneal (i.p.) injection for 15 days, the survival and body weights of the mice were recorded after treatment.

FIG. 6 illustrates the ¹H NMR spectrum of compound 1a.

FIG. 7 illustrates the ¹³C NMR spectrum of compound 1a.

FIG. 8 illustrates the ¹H NMR spectrum of compound 1b.

FIG. 9 illustrates the ¹³C NMR spectrum of compound 1b.

FIG. 10 illustrates the ¹H NMR spectrum of compound 1c.

FIG. 11 illustrates the ¹³C NMR spectrum of compound 1c.

FIG. 12 illustrates the anticancer effects and selectivity of 6MP (10 μM), prodrugs YA6131 (10 μM) and YA6075 (10 μM), evaluated in human colorectal cancer cells SW480 and normal HEK293 cells.

### DETAILED DESCRIPTION OF THE INVENTION

The disclosure relates generally to anti-cancer or anti-autoimmune diseases prodrugs, including but not limited to reactive ROS-activated prodrugs, and compositions and methods of reducing side effects in chemotherapies. Without wishing to be bound by any particular theory, the disclosure is based at least on part on the observations that elevated levels of ROS have been detected in almost all cancers.

Without wishing to be bound by any particular theory, it is believed that the novel prodrugs described herein, including without limitation arylboronate-based 5-FU prodrugs in this disclosure, are activated by elevated levels of ROS in cancer cells, releasing 5-FU. As described herein, the arylboronate 5-FU prodrugs exhibit high specificity for cancer cells over normal cells. Furthermore, the arylboronate 5-FU prodrugs demonstrate a more favorable safety profile compared to parent 5-FU. The ROS-activated prodrugs offer effective ways to improve the therapeutic effectiveness and selectivity of 5-FU in anticancer chemotherapies.

Since its introduction more than 40 years ago, 5-FU has become a component of the standard therapy for a varieties of malignancies. 5-FU is a small molecule with a pKa of 8.0, which should predict excellent absorption and bioavailability. However, the use of oral 5-FU was abandoned decades ago because of its irregular absorption. Plasma level of 5-FU are quite unpredictable after oral administration with marked intra- and inter-individual difference due to variable activity of pyrimidine degrading enzyme dihydropyrimidine dehydrogenase (DPD). 5-FU efficacy is further limited by the short t_{1/2} of 5-FU in plasma, and its resistance of some tumors with strong expression of TS or low reserves of reduced folates. In addition, the mode of administration is also problematic. Any i.v. administration whether by bolus or continuous infusion, requires the presence of the patient in hospital and also presents risk of complications (e.g. venous thrombosis, or infection around the catheter).

Additionally, while remarkably benefiting cancer patients, 5-FU also indicates significant side effects such as myelosuppression, central neurotoxicity, and gastrointestinal toxicity. Moreover, 5-FU is metabolically unstable with the majority of the dose converted to 3-fluoroalanine DPD enzyme.

### Definitions

Unless defined otherwise, all technical and scientific terms used herein have the same meaning as is commonly understood by one of skill in the art to which this disclosure belongs. All patents and publications referred to herein are incorporated by reference in their entireties.

As used herein, the terms “treat,” “treatment,” and/or “treating” may refer to the management of a disease, disorder, or pathological condition, or symptom thereof with the intent to cure, ameliorate, stabilize, prevent, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition).

The term “in vivo” refers to an event that takes place in a subject's body. The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application, including in vitro or in vivo, or the subject and disease condition being treated (e.g., the weight, age, and gender of the subject), the severity of the disease condition, the manner of administration, etc., which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs, can also be incorporated into the described compositions and methods.

“Prodrug” is intended to describe a compound that may be converted under physiological conditions, or by solvolysis, to a biologically active compound, including a compound described herein. Thus, the term “prodrug” refers to a precursor of a biologically active compound that is pharmaceutically acceptable. A prodrug may be inactive when administered to a subject, but is converted in vivo to an active compound, for example, by hydrolysis, or by ROS activation. The prodrug compound often offers the advantages of solubility, tissue compatibility or delayed release in a mammalian organism (see, e.g., Bundgaard, H., Design of Prodrugs, 1985, Elsevier, Amsterdam). The term “prodrug” is also intended to include any covalently bonded carriers, which release the active compound in vivo when administered to a subject. Prodrugs of an active compound, as described herein, may be prepared by modifying functional groups present in the active compound in such a way that the modifications are cleaved, either in routine manipulation or in vivo, to yield the active parent compound. Prodrugs include, for example, compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetates, formates, and benzoate derivatives of an alcohol, various ester derivatives of a carboxylic acid, or acetamide, formamide, and benzamide derivatives of an amine functional group in the active compound. Prodrugs include N-alkyl compounds, wherein the alkyl is optionally substituted. Prodrugs include N-benzyl compounds.

As used herein, the terms “programmed cell death” and “apoptosis” are used interchangeably.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this invention.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, or from 0% to 10%, or from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Alkyl” refers to a straight or branched hydrocarbon chain radical consisting solely of carbon and hydrogen atoms, containing no unsaturation. In some embodiments, an alkyl can have from one to ten carbon atoms (e.g., (C₁₋₁₀)alkyl or C₁₋₁₀ alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl), and 3-methylhexyl. Unless stated otherwise specifically in the specification, an alkyl group is optionally substituted by one or more of substituents which are independently heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), —S(O)ₜN(R^{a})C(O)R^{b} (where t is 1 or 2), or —PO(OR^{a})₂, where each R^{a} is independently hydrogen, alkyl, alkenyl, fluoroalkyl, carbocyclyl, carbocyclylalkyl, aryl, aralkyl, heterocycloalkyl, heterocycloalkylalkyl, heteroaryl, or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocyclyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond. In some embodiments, an alkenyl group has from two to ten carbon atoms (i.e., (C₂₋₁₀)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), —S(O)ₜN(R^{a})C(O)R^{b} (where t is 1 or 2), or —PO(OR^{a})₂, where each R^{a} is independently hydrogen, alkyl, alkenyl, fluoroalkyl, carbocyclyl, carbocyclylalkyl, aryl, aralkyl, heterocycloalkyl, heterocycloalkylalkyl, heteroaryl, or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond. In some embodiments, an alkynyl group has from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), —S(O)ₜN(R^{a})C(O)R^{b} (where t is 1 or 2), or —PO(OR^{a})₂, where each R^{a} is independently hydrogen, alkyl, alkenyl, fluoroalkyl, carbocyclyl, carbocyclylalkyl, aryl, aralkyl, heterocycloalkyl, heterocycloalkylalkyl, heteroaryl, or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a —(C═O)H radical.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. (C₃₋₁₀)cycloalkyl or C₃₋₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), —S(O)ₜN(R^{a})C(O)R^{b} (where t is 1 or 2), or —PO(OR^{a})₂, where each R^{a} is independently hydrogen, alkyl, alkenyl, fluoroalkyl, carbocyclyl, carbocyclylalkyl, aryl, aralkyl, heterocycloalkyl, heterocycloalkylalkyl, heteroaryl, or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), —S(O)ₜN(R^{a})C(O)R^{b} (where t is 1 or 2), or —PO(OR^{a})₂, where each R^{a} is independently hydrogen, alkyl, alkenyl, fluoroalkyl, carbocyclyl, carbocyclylalkyl, aryl, aralkyl, heterocycloalkyl, heterocycloalkylalkyl, heteroaryl, or heteroarylalkyl.

The term “alkoxycarbonyl” refers to a group of the formula (alkoxy)(C═O)— attached through the carbonyl carbon wherein the alkoxy group has the indicated number of carbon atoms. Thus a (C₁₋₆)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), —S(O)ₜN(R^{a})C(O)R^{b} (where t is 1 or 2), or —PO(OR^{a})₂, where each R^{a} is independently hydrogen, alkyl, alkenyl, fluoroalkyl, carbocyclyl, carbocyclylalkyl, aryl, aralkyl, heterocycloalkyl, heterocycloalkylalkyl, heteroaryl, or heteroarylalkyl.

“Acyl” refers to the groups (alkyl)-C(O)—, (aryl)-C(O)—, (heteroaryl)-C(O)—, (heteroalkyl)-C(O)— and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), —S(O)ₜN(R^{a})C(O)R^{b} (where t is 1 or 2), or —PO(OR^{a})₂, where each R^{a} is independently hydrogen, alkyl, alkenyl, fluoroalkyl, carbocyclyl, carbocyclylalkyl, aryl, aralkyl, heterocycloalkyl, heterocycloalkylalkyl, heteroaryl, or heteroarylalkyl.

“Acyloxy” refers to a R(C═O)O— radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), —S(O)ₜN(R^{a})C(O)R^{b} (where t is 1 or 2), or —PO(OR^{a})₂, where each R^{a} is independently hydrogen, alkyl, alkenyl, fluoroalkyl, carbocyclyl, carbocyclylalkyl, aryl, aralkyl, heterocycloalkyl, heterocycloalkylalkyl, heteroaryl, or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), —S(O)ₜN(R^{a})C(O)R^{b} (where t is 1 or 2), —S(O)ₜN(R^{a})C(O)R^{b} (where t is 1 or 2), or —PO(OR^{a})₂, where each R^{a} is independently hydrogen, alkyl, alkenyl, fluoroalkyl, carbocyclyl, carbocyclylalkyl, aryl, aralkyl, heterocycloalkyl, heterocycloalkylalkyl, heteroaryl, or heteroarylalkyl.

The term “substituted amino” also refers to N-oxides of the groups —NHR^{d}, and NR^{d}R^{d} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R^{a})₂ or —NHC(O)R^{a}, where each R^{a} is independently selected from the group consisting of hydrogen, alkyl, alkenyl, cycloalkyl, aryl, heteroaryl (bonded through a ring carbon), and heteroalicyclic (bonded through a ring carbon), each of which moiety may itself be optionally substituted. The (R^{a})₂ of —N(R^{a})₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), —S(O)ₜN(R^{a})C(O)R^{b} (where t is 1 or 2), or —PO(OR^{a})₂, where each R^{a} is independently hydrogen, alkyl, alkenyl, fluoroalkyl, carbocyclyl, carbocyclylalkyl, aryl, aralkyl, heterocycloalkyl, heterocycloalkylalkyl, heteroaryl, or heteroarylalkyl.

The term “aryloxy” refers to the group —O-aryl.

The term “substituted aryloxy” refers to aryloxy wherein the aryl substituent is substituted (i.e., —O-(substituted aryl)). Unless stated otherwise specifically in the specification, the aryl moiety of an aryloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), —S(O)ₜN(R^{a})C(O)R^{b} (where t is 1 or 2), or —PO(OR^{a})₂, where each R^{a} is independently hydrogen, alkyl, alkenyl, fluoroalkyl, carbocyclyl, carbocyclylalkyl, aryl, aralkyl, heterocycloalkyl, heterocycloalkylalkyl, heteroaryl, or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, alkenyl, cycloalkyl, aryl, heteroaryl (bonded through a ring carbon), and heteroalicyclic (bonded through a ring carbon). The procedures and specific groups to make esters are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), —S(O)ₜN(R^{a})C(O)R^{b} (where t is 1 or 2), or —PO(OR^{a})₂, where each R^{a} is independently hydrogen, alkyl, alkenyl, fluoroalkyl, carbocyclyl, carbocyclylalkyl, aryl, aralkyl, heterocycloalkyl, heterocycloalkylalkyl, heteroaryl, or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo, or iodo. The terms “haloalkyl,” “haloalkenyl,” “haloalkynyl,” and “haloalkoxy” include alkyl, alkenyl, alkynyl, and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), —S(O)ₜN(R^{a})C(O)R^{b} (where t is 1 or 2), or —PO(OR^{a})₂, where each R^{a} is independently hydrogen, alkyl, alkenyl, fluoroalkyl, carbocyclyl, carbocyclylalkyl, aryl, aralkyl, heterocycloalkyl, heterocycloalkylalkyl, heteroaryl, or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom.

The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-TH-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e., thienyl). Unless stated otherwise specifically in the specification, a heteroaryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), —S(O)ₜN(R^{a})C(O)R^{b} (where t is 1 or 2), or —PO(OR^{a})₂, where each R^{a} is independently hydrogen, alkyl, alkenyl, fluoroalkyl, carbocyclyl, carbocyclylalkyl, aryl, aralkyl, heterocycloalkyl, heterocycloalkylalkyl, heteroaryl, or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), —S(O)ₜN(R^{a})C(O)R^{b} (where t is 1 or 2), or —PO(OR^{a})₂, where each R^{a} is independently hydrogen, alkyl, alkenyl, fluoroalkyl, carbocyclyl, carbocyclylalkyl, aryl, aralkyl, heterocycloalkyl, heterocycloalkylalkyl, heteroaryl, or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Nitro” refers to the —NO₂ radical.

“Oxa” refers to the —O— radical.

“Oxo” refers to the ═O radical.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In some embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, Third Edition, John Wiley & Sons, New York (1999).

“Solvate” refers to a compound in physical association with one or more molecules of a pharmaceutically acceptable solvent.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S— (optionally substituted alkyl), —S— (optionally substituted aryl), —S— (optionally substituted heteroaryl) and —S— (optionally substituted heterocycloalkyl).

“Sulfinyl” refers to groups that include —S(O)—H, —S(O)— (optionally substituted alkyl), —S(O)— (optionally substituted amino), —S(O)— (optionally substituted aryl), —S(O)— (optionally substituted heteroaryl) and —S(O)— (optionally substituted heterocycloalkyl).

“Sulfonyl” refers to groups that include —S(O₂)—H, —S(O₂)— (optionally substituted alkyl), —S(O₂)— (optionally substituted amino), —S(O₂)— (optionally substituted aryl), —S(O₂)— (optionally substituted heteroaryl), and —S(O₂)— (optionally substituted heterocycloalkyl).

“Sulfonamidyl” or “sulfonamido” refers to a —S(O)₂N(R^{a})₂ radical, where each R^{a} is selected independently from the group consisting of hydrogen, alkyl, alkenyl, cycloalkyl, aryl, heteroaryl (bonded through a ring carbon) and heteroalicyclic (bonded through a ring carbon). The R^{a} groups in —N(R^{a})₂ of the —S(O)₂N(R^{a})₂ radical may be taken together with the nitrogen to which it is attached to form a 4-, 5-, 6- or 7-membered ring. A sulfonamido group is optionally substituted by one or more of the substituents described for alkyl, alkenyl, cycloalkyl, aryl, heteroaryl, respectively.

“Sulfoxyl” refers to a —S(O)₂OH radical.

“Sulfonate” refers to a —S(O)₂OR radical, where R is selected from the group consisting of alkyl, alkenyl, cycloalkyl, aryl, heteroaryl (bonded through a ring carbon) and heteroalicyclic (bonded through a ring carbon). A sulfonate group is optionally substituted on R by one or more of the substituents described for alkyl, alkenyl, cycloalkyl, aryl, heteroaryl, respectively.

Compounds of the invention also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

The term “hematological malignancy” refers to mammalian cancers and tumors of the hematopoietic and lymphoid tissues, including but not limited to tissues of the blood, bone marrow, lymph nodes, and lymphatic system. Hematological malignancies are also referred to as “liquid tumors.” Hematological malignancies include, but are not limited to, ALL, CLL, SLL, acute myelogenous leukemia (AML), chronic myelogenous leukemia (CML), acute monocytic leukemia (AMoL), Hodgkin's lymphoma, and non-Hodgkin's lymphomas. The term “B cell hematological malignancy” refers to hematological malignancies that affect B cells.

The term “solid tumor” refers to an abnormal mass of tissue that usually does not contain cysts or liquid areas. Solid tumors may be benign or malignant. The term “solid tumor cancer” refers to malignant, neoplastic, or cancerous solid tumors. Solid tumor cancers include, but are not limited to, sarcomas, carcinomas, and lymphomas, such as cancers of the lung, breast, prostate, colon, rectum, and bladder. The tissue structure of solid tumors includes interdependent tissue compartments including the parenchyma (cancer cells) and the supporting stromal cells in which the cancer cells are dispersed and which may provide a supporting microenvironment.

For the avoidance of doubt, it is intended herein that particular features (for example integers, characteristics, values, uses, diseases, formulae, compounds or groups) described in conjunction with a particular aspect, embodiment or example of the invention are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any disclosed embodiments. The invention extends to any novel one, or novel combination, of the features disclosed in this specification (including any accompanying claims, abstract and drawings), or to any novel one, or any novel combination, of the steps of any method or process so disclosed.

### 5-Fluorouracil Prodrugs and Reactive Oxygen Species

To overcome the limitation of 5-FU, prodrug strategies have been actively pursued in the past three decades. Among numerous small molecule 5-FU-prodrugs described in the literature, four have been successfully used in the clinic including tegafur, doxifluridine, carmofur, and capecitabine. These compounds are orally available, and activated in human body via different mechanisms. Activation of tegafur involves either a 5′-hydroxylation reaction catalyzed by the cytochrome P450 enzyme CYP2A6, or an enzymatic cleavage of the N1-C2′ bond of the molecule. Doxifluridine is a second generation of nucleotide-based prodrug of 5-FU that have been approved in several Asian countries. Its activation in tumors is achieved by either thymidine phosphorylase or pyrimidine-nucleoside phosphorylase. The lipophilic 5-FU analog carmofur overcomes the degradation problem of 5-FU by DPD. The carbamoyl group of carmofur is enzymatically removed in human body to release 5-FU. Capecitabine is another carbamate-based 5-FU-prodrug that is activated in the liver and cancer cells through a sequential reaction catalyzed by carboxylesterase and cytidine deaminase.

Reactive oxygen species (ROS), the metabolic byproduct of oxygen metabolism, play important role in maintaining cellular redox homeostasis. Unlike the normal cell environment where the ROS level is controlled by balancing its production and elimination, cancer cells exhibit enhanced levels of ROS such as superoxide (O₂.⁻), hydrogen peroxide (H₂O₂), and hydroxyl radical (HO⁻), due to increased metabolic activity and mitochondrial malfunction. It has been shown that high levels of ROS in cancer cells are also associated with DNA mutations that lead to tumor cell angiogenesis and metastasis, and drug resistance. At the same time, growth and transformation of cancer cells are promoted with the condition of a modest rise of intracellular ROS.

While ROS itself has been exploited for the development of tumor-selective therapeutics by amplifying oxidative stress, ROS-activated prodrugs can also be developed to obtain tumor selective agents. In some embodiments, the present disclosure provides ROS activated 5-FU prodrugs to diminish or circumvent some the limitation of 5-FU in chemotherapy such as reduction in toxicity by targeting tumor site. In some embodiments, the disclosure provides ROS 5-FU prodrugs that liberate the active principle 5-FU selectively in tumor cells with elevated levels of ROS (Scheme 1).

In some embodiments, the disclosure relates to the design and evaluation of novel arylboronate-based 5-FU prodrugs 1a-1j (Scheme 1). The p-boronate-benzyl group was introduced to the N1 position of 5-FU, to limit the metabolic degradation by DPD. The arylboronate groups are designed as ROS-sensitive triggers, which react with a ROS, for example H₂O₂, to generate the corresponding phenol intermediate. Spontaneous breakdown of this intermediate will release 5-FU, along with 4-methylenecyclohexa-2,5-dien-1-one as a byproduct (Scheme 1):

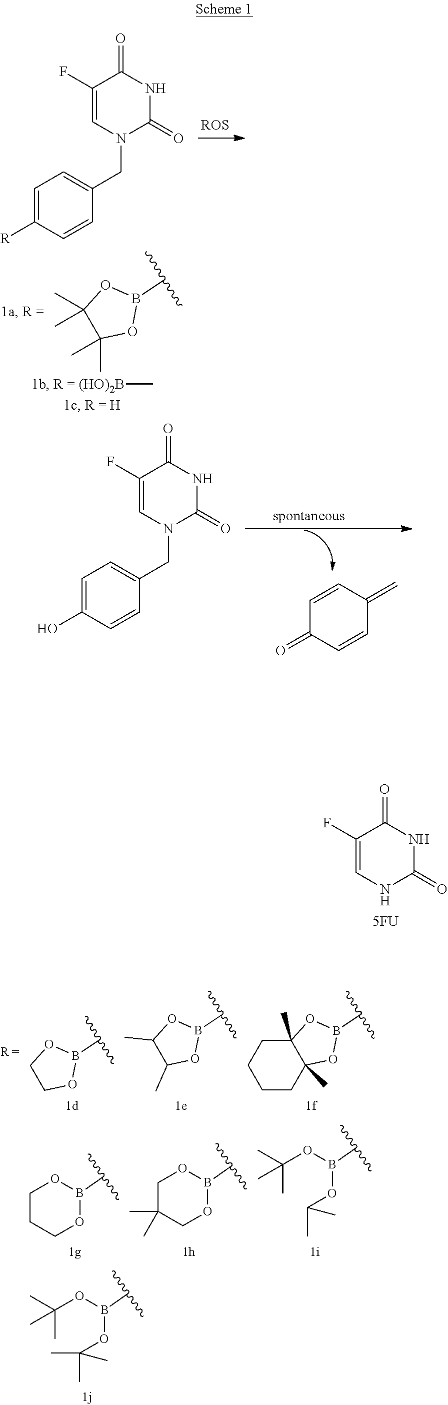

In some embodiments, the disclosure provides a compound of Formula I, or a pharmaceutically acceptable salt, solvate, hydrate, cocrystal, or prodrug thereof:

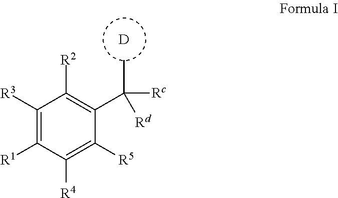

wherein in Formula I: D is a drug or prodrug moiety comprising at least one heterocycle; R¹, R², R³, R⁴, and R⁵ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted alkynyl, substituted or unsubstituted heteroalkynyl, substituted or unsubstituted cycloalkynyl, substituted or unsubstituted heterocycloalkynyl, substituted or unsubstituted aryl, substituted or unsubstituted heteroaryl, hydroxy, halo, cyano, trifluoromethyl, trifluoromethoxy, nitro, trimethylsilanyl, —OR^{a}, —B(OR^{a})OR^{b}, —SR^{a}, —OC(O)—R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{b}, —N(R^{a})C(O)R^{b}, —N(R^{a})C(O)N(R^{a})R^{b}, —N(R^{a})C(NR^{a})N(R^{a})R^{b}, —N(R^{a})S(O)ₜR^{b}, —S(O)ₜR^{a}, —S(O)ₜOR^{a}, —S(O)ₜN(R^{a})R^{b}, and —PO(OR^{a})(OR^{b}), wherein at least one of R¹, R², R³, R⁴, or R⁵ is —OR^{a} or —B(OR^{a})OR^{b}; R^{a}, R^{b}, R^{c}, and R^{d} are independently selected at each occurrence from hydrogen, substituted or unsubstituted alkyl, substituted or unsubstituted fluoroalkyl, substituted or unsubstituted carbocyclyl, substituted or unsubstituted carbocyclylalkyl, substituted or unsubstituted aryl, substituted or unsubstituted aralkyl, substituted or unsubstituted heterocycloalkyl, substituted or unsubstituted heterocycloalkylalkyl, substituted or unsubstituted heteroaryl, or substituted or unsubstituted heteroarylalkyl; t is 1 or 2; wherein any of R¹, R², R³, R⁴, R⁵, R^{a}, and R^{b} can optionally be linked together.

In some embodiments, the disclosure provides a compound of Formula I, wherein R¹ is —OR^{a} or —B(OR^{a})OR^{b}. In some embodiments, R² is —OR^{a} or —B(OR^{a})OR^{b}. In some embodiments, R⁵ is —OR^{a} or —B(OR^{a})OR^{b}.

In some embodiments, the disclosure provides a compound of any one of Formula 11, Formula 12, Formula 13, Formula 14, or Formula 15:

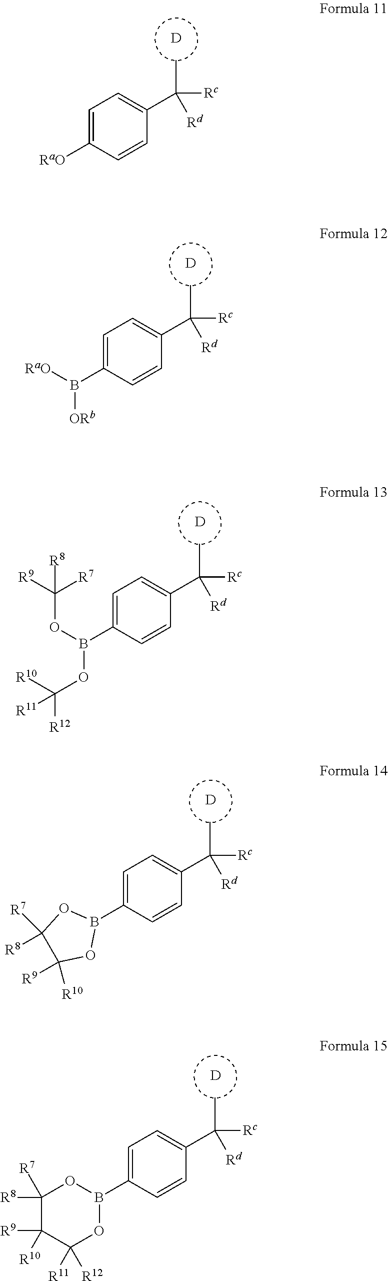

wherein in Formulas 11-15: R⁷, R⁸, R⁹, R¹⁰, R¹¹, and R¹² are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted alkynyl, substituted or unsubstituted heteroalkynyl, substituted or unsubstituted cycloalkynyl, substituted or unsubstituted heterocycloalkynyl, substituted or unsubstituted aryl, substituted or unsubstituted heteroaryl, hydroxy, halo, cyano, trifluoromethyl, trifluoromethoxy, nitro, trimethylsilanyl, —OR^{a}, —SR^{a}, —OC(O)—R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{b}, —N(R^{a})C(O)R^{b}, —N(R^{a})C(O)N(R^{a})R^{b}, —N(R^{a})C(NR^{a})N(R^{a})R^{b}, —N(R^{a})S(O)ₜR^{b}, —S(O)ₜR^{a}, —S(O)ₜOR^{a}, —S(O)ₜN(R^{a})R^{b}, and —PO(OR^{a})(OR^{b}); R^{a}, R^{b}, R^{c}, and R^{d} are independently selected from hydrogen, substituted or unsubstituted alkyl, substituted or unsubstituted fluoroalkyl, substituted or unsubstituted carbocyclyl, substituted or unsubstituted carbocyclylalkyl, substituted or unsubstituted aryl, substituted or unsubstituted aralkyl, substituted or unsubstituted heterocycloalkyl, substituted or unsubstituted heterocycloalkylalkyl, substituted or unsubstituted heteroaryl, or substituted or unsubstituted heteroarylalkyl; t is 1 or 2; and wherein any of R⁷, R⁸, R⁹, R¹⁰, R¹¹, R¹², R^{a}, and R^{b} can optionally be linked together.

In some embodiments, the disclosure provides compounds of Formula I, or any one of Formula 11, Formula 12, Formula 13, Formula 14, or Formula 15, wherein: R⁷, R⁸, R⁹, R¹⁰, R¹¹, R¹², R^{a}, and R^{b} are independently selected from hydrogen, and substituted or unsubstituted alkyl. In some embodiments, at least one of R^{c} and R^{d} is hydrogen. In some embodiments, both R^{c} and R^{d} are hydrogen.

In some embodiments, the disclosure provides a compound of any one of the following Formulas, wherein D is defined as described herein:

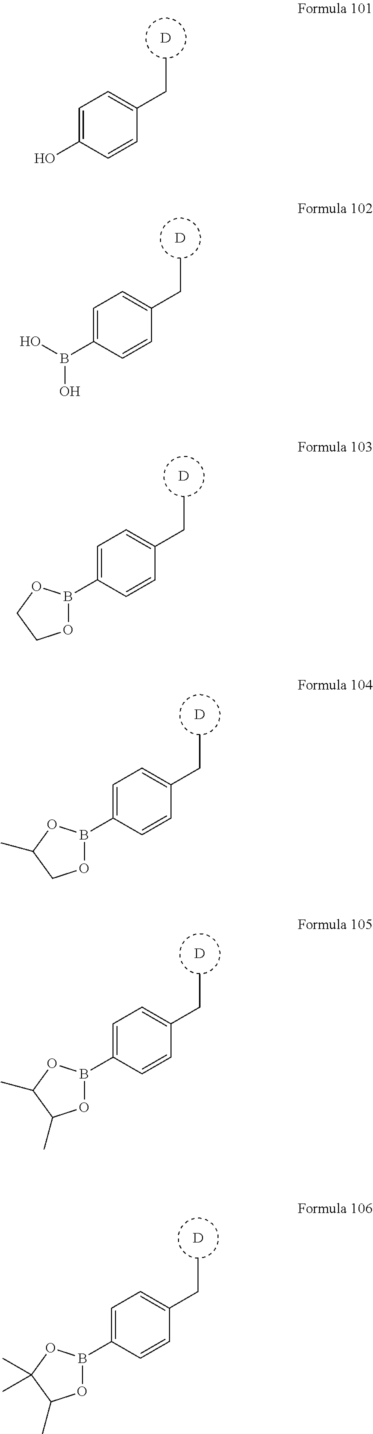

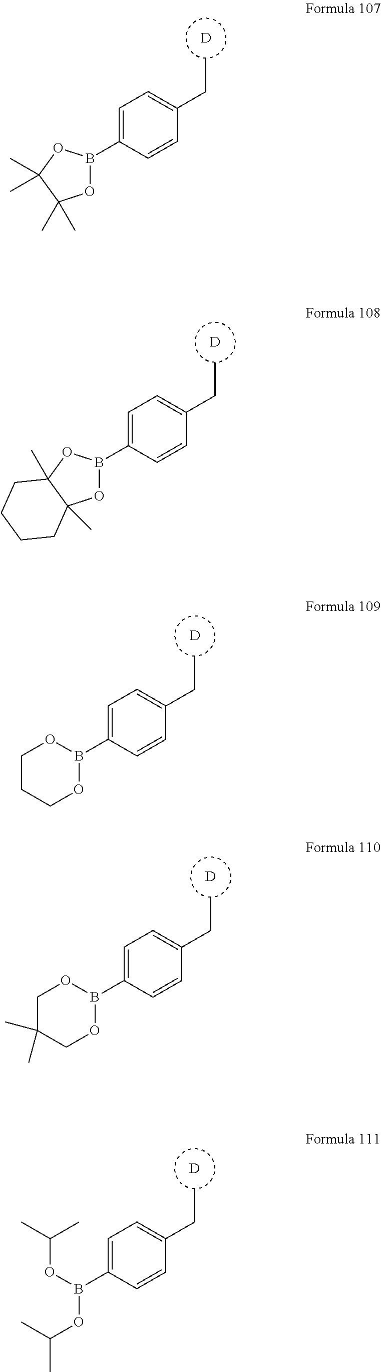

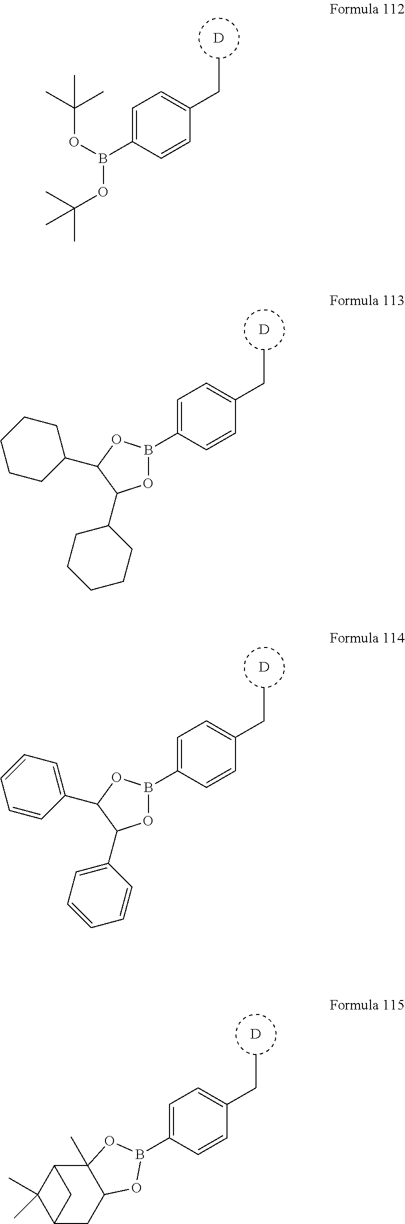

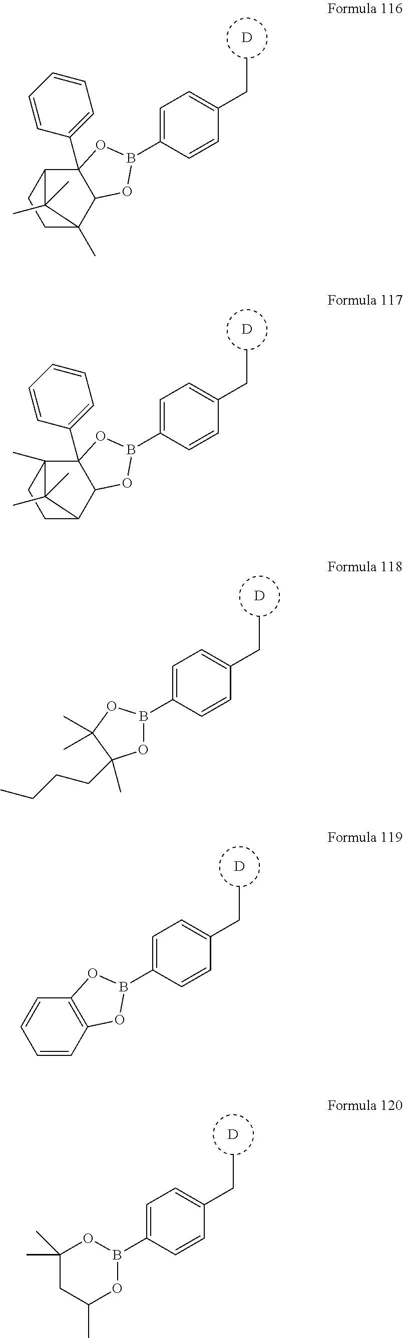

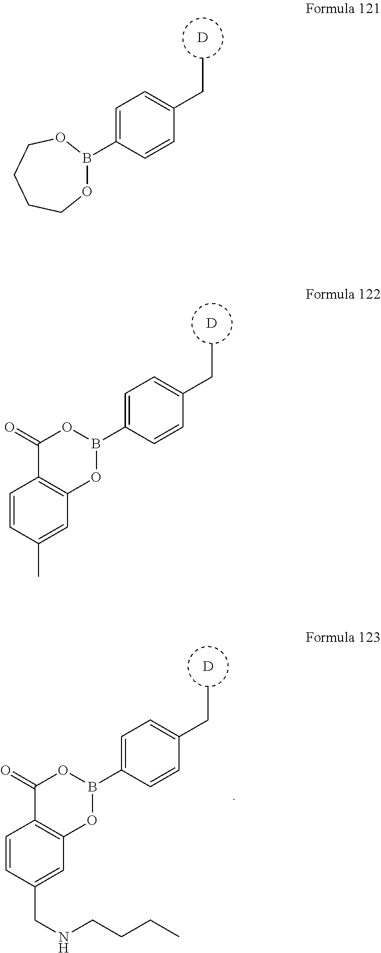

In some embodiments, the disclosure provides compounds of Formula I, or any one of Formula 11, Formula 12, Formula 13, Formula 14, or Formula 15, or any one of Formulas 101 to 123, wherein D comprises a pyrimidine moiety.

In some embodiments, the disclosure provides compounds of Formula I, or any one of Formula 11, Formula 12, Formula 13, Formula 14, or Formula 15, or any one of Formulas 101 to 123, wherein D is

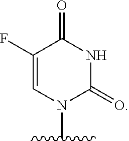 [SMILES: *n1cc(F)c(=O)[nH]c1=O]

In some embodiments, the disclosure provides compounds of Formula I, or any one of Formula 11, Formula 12, Formula 13, Formula 14, or Formula 15, or any one of Formulas 101 to 123, wherein D is

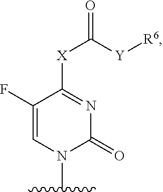

and wherein X and Y are independently selected from —O— and —NR^{a}—, and R⁶ is selected from hydrogen, substituted or unsubstituted alkyl, and substituted or unsubstituted heteroalkyl.

In some embodiments, the disclosure provides compounds of Formula I, or any one of Formula 11, Formula 12, Formula 13, Formula 14, or Formula 15, or any one of Formulas 101 to 123, wherein D comprises a purine moiety. In some embodiments, D is

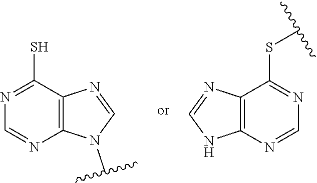 [SMILES: *n1cnc2c(S)ncnc21 ; *Sc1ncnc2[nH]cnc12]

In some embodiments, the disclosure provides compounds of Formula I, or any one of Formula 11, Formula 12, Formula 13, Formula 14, or Formula 15, or any one of Formulas 101 to 123, wherein D comprises a moiety selected from:

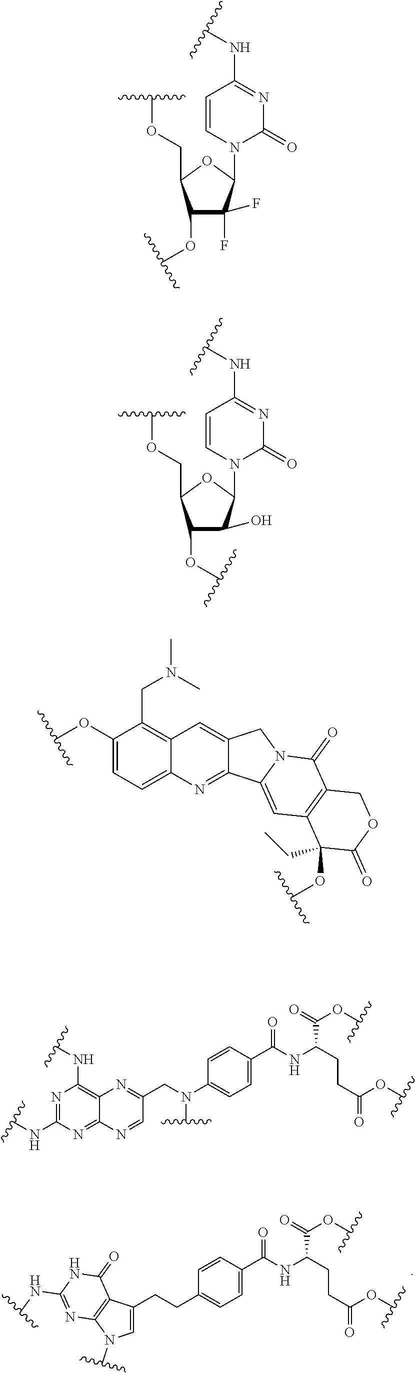

In some embodiments, the disclosure provides compounds of Formula I, or any one of Formula 11, Formula 12, Formula 13, Formula 14, or Formula 15, or any one of Formulas 101 to 123, wherein D is selected from:

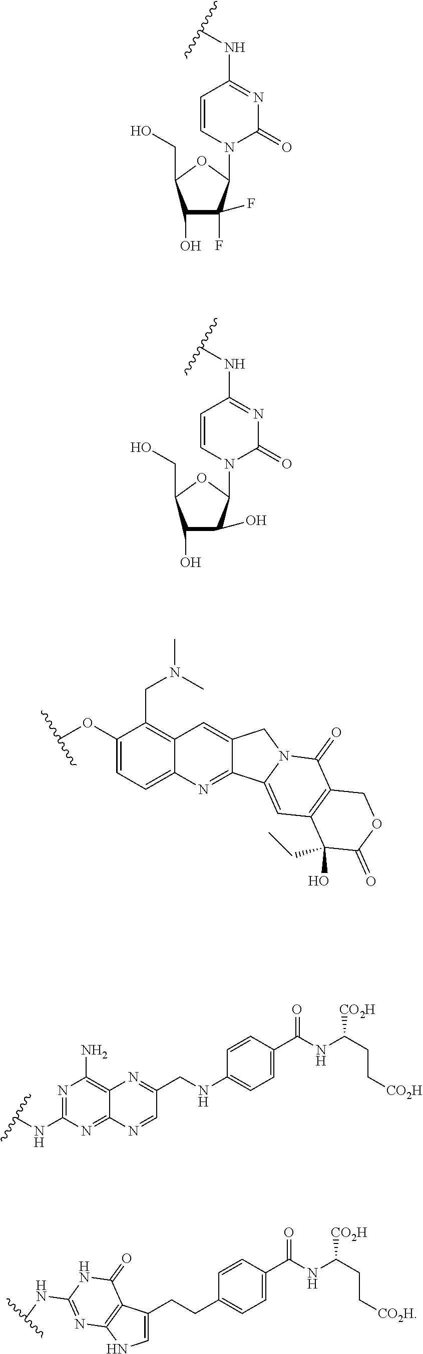

In some embodiments, the disclosure provides compounds of Formula I, or any one of Formula 11, Formula 12, Formula 13, Formula 14, or Formula 15, or any one of Formulas 101 to 123, wherein D is selected from gemcitabine, methotrexate, cytarabine, pemetrexed, and topotecan.

In some embodiments, the disclosure provides a compound of Formula A, Formula B, Formula C, or Formula D, or a pharmaceutically acceptable salt, solvate, hydrate, cocrystal, or prodrug thereof:

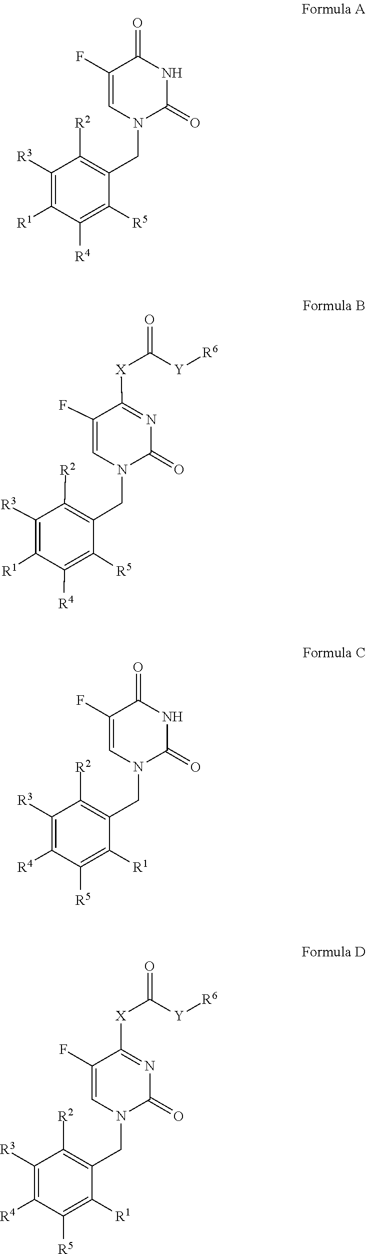

wherein in Formulas A-D: R¹ is —OR^{a} or —B(OR^{a})OR^{b}; X and Y are independently selected from —O— and —NR^{a}—; and R², R³, R⁴, R⁵, and R⁶ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted alkynyl, substituted or unsubstituted heteroalkynyl, substituted or unsubstituted cycloalkynyl, substituted or unsubstituted heterocycloalkynyl, substituted or unsubstituted aryl, substituted or unsubstituted heteroaryl, hydroxy, halo, cyano, trifluoromethyl, trifluoromethoxy, nitro, trimethylsilanyl, —OR^{a}, —SR^{a}, —OC(O)—R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{b}, —N(R^{a})C(O)R^{b}, —N(R^{a})C(O)N(R^{a})R^{b}, —N(R^{a})C(NR^{a})N(R^{a})R^{b}, —N(R^{a})S(O)ₜR^{b}, —S(O)ₜR^{a} (where t is 1 or 2), —S(O)ₜOR^{a}, —S(O)ₜN(R^{a})R^{b}, and —PO(OR^{a})(OR^{b}); R^{a} and R^{b} are independently selected from hydrogen, substituted or unsubstituted alkyl, substituted or unsubstituted fluoroalkyl, substituted or unsubstituted carbocyclyl, substituted or unsubstituted carbocyclylalkyl, substituted or unsubstituted aryl, substituted or unsubstituted aralkyl, substituted or unsubstituted heterocycloalkyl, substituted or unsubstituted heterocycloalkylalkyl, substituted or unsubstituted heteroaryl, or substituted or unsubstituted heteroarylalkyl; t is 1 or 2; and wherein any of R², R³, R⁴, R⁵, R⁶, R^{a}, and R^{b} can optionally be linked together.

In some embodiments, the disclosure provides a compound of any one of Formulas 16 to 25:

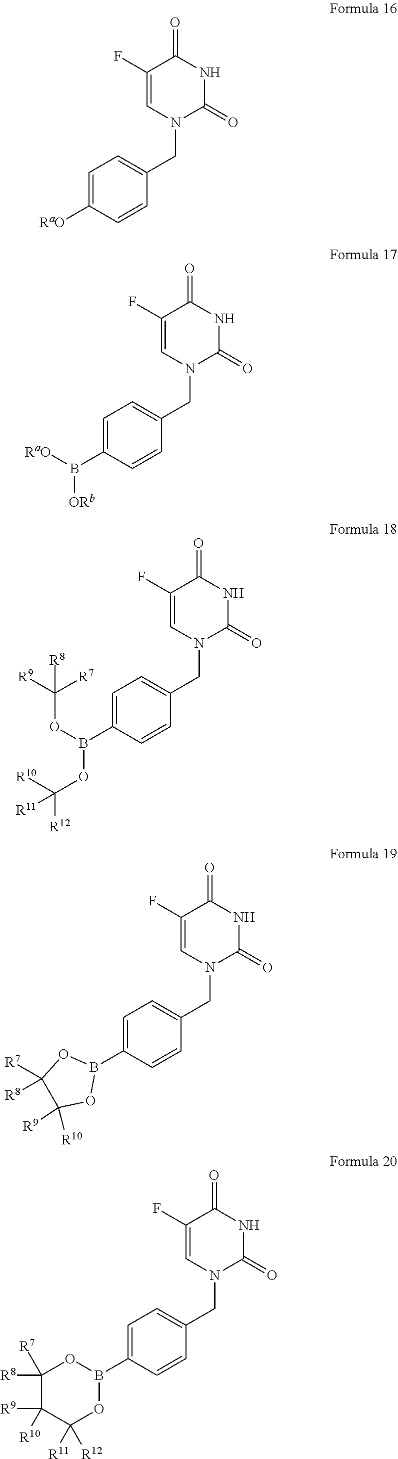

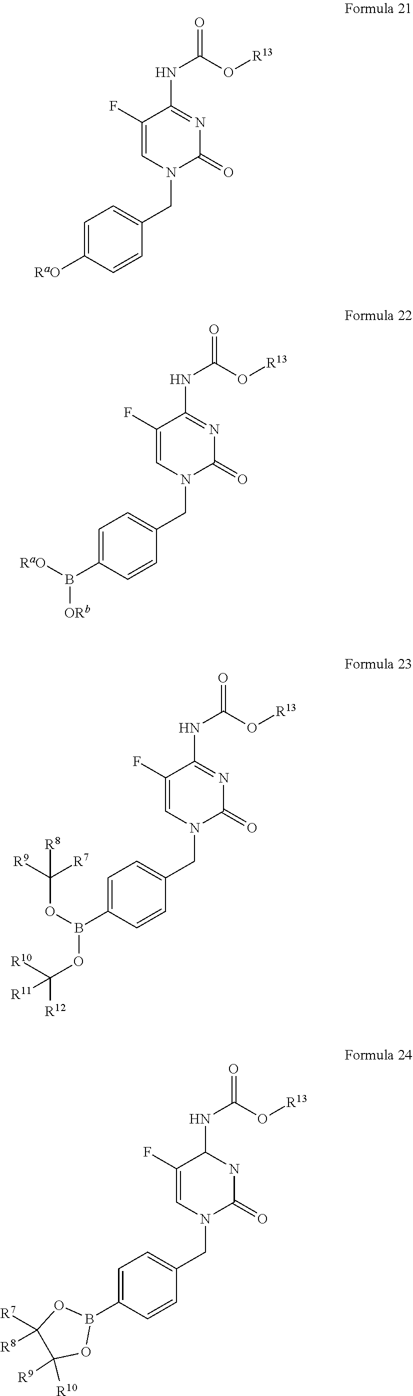

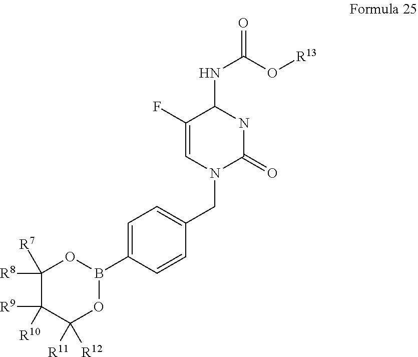

wherein in Formulas 16 to 25: R⁷, R⁸, R⁹, R¹⁰, R¹¹, R¹², and R¹³ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted alkynyl, substituted or unsubstituted heteroalkynyl, substituted or unsubstituted cycloalkynyl, substituted or unsubstituted heterocycloalkynyl, substituted or unsubstituted aryl, substituted or unsubstituted heteroaryl, hydroxy, halo, cyano, trifluoromethyl, trifluoromethoxy, nitro, trimethylsilanyl, —OR^{a}, —SR^{a}, —OC(O)—R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{b}, —N(R^{a})C(O)R^{b}, —N(R^{a})C(O)N(R^{a})R^{b}, —N(R^{a})C(NR^{a})N(R^{a})R^{b}, —N(R^{a})S(O)ₜR^{b}, —S(O)ₜR^{a} (where t is 1 or 2), —S(O)ₜOR^{a}, —S(O)ₜN(R^{a})R^{b}, and —PO(OR^{a})(OR^{b}); R^{a} and R^{b} are independently selected from hydrogen, substituted or unsubstituted alkyl, substituted or unsubstituted fluoroalkyl, substituted or unsubstituted carbocyclyl, substituted or unsubstituted carbocyclylalkyl, substituted or unsubstituted aryl, substituted or unsubstituted aralkyl, substituted or unsubstituted heterocycloalkyl, substituted or unsubstituted heterocycloalkylalkyl, substituted or unsubstituted heteroaryl, or substituted or unsubstituted heteroarylalkyl; t is 1 or 2; and wherein any of R⁷, R⁸, R⁹, R¹⁰, R¹¹, R¹², R^{a}, and R^{b} can optionally be linked together. In some embodiments, the disclosure provides a compound of any one of Formulas 16 to 25, wherein: R⁷, R⁸, R⁹, R¹⁰, R¹¹, R¹², R¹³, R^{a}, and R^{b} are independently selected from hydrogen, and substituted or unsubstituted alkyl.

In some embodiments, the invention relates to a compound of any one of Formulas 1001-1023:

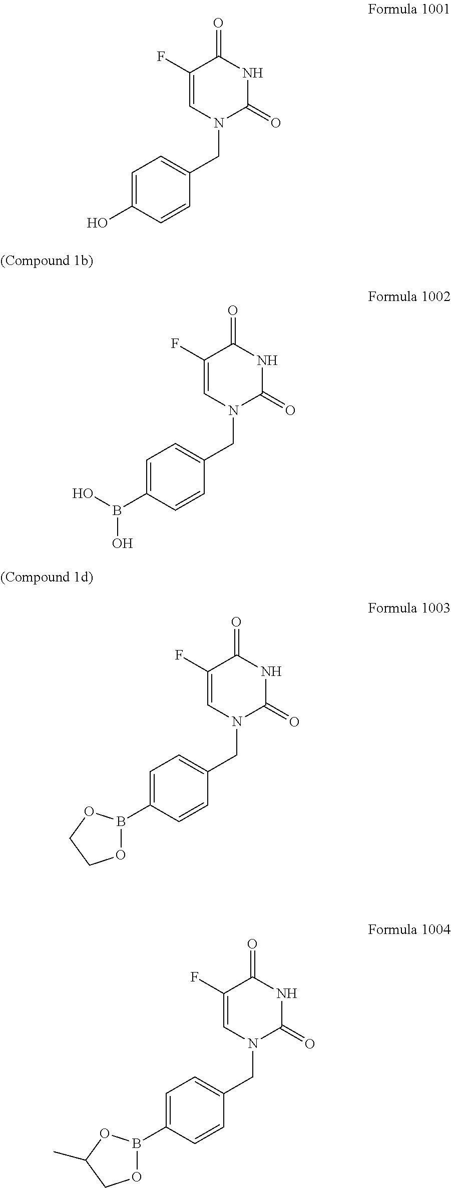 [SMILES: O=c1[nH]c(=O)n(Cc2ccc(O)cc2)cc1F ; O=c1[nH]c(=O)n(Cc2ccc(B(O)O)cc2)cc1F ; O=c1[nH]c(=O)n(Cc2ccc(B3OCCO3)cc2)cc1F ; CC1COB(c2ccc(Cn3cc(F)c(=O)[nH]c3=O)cc2)O1]

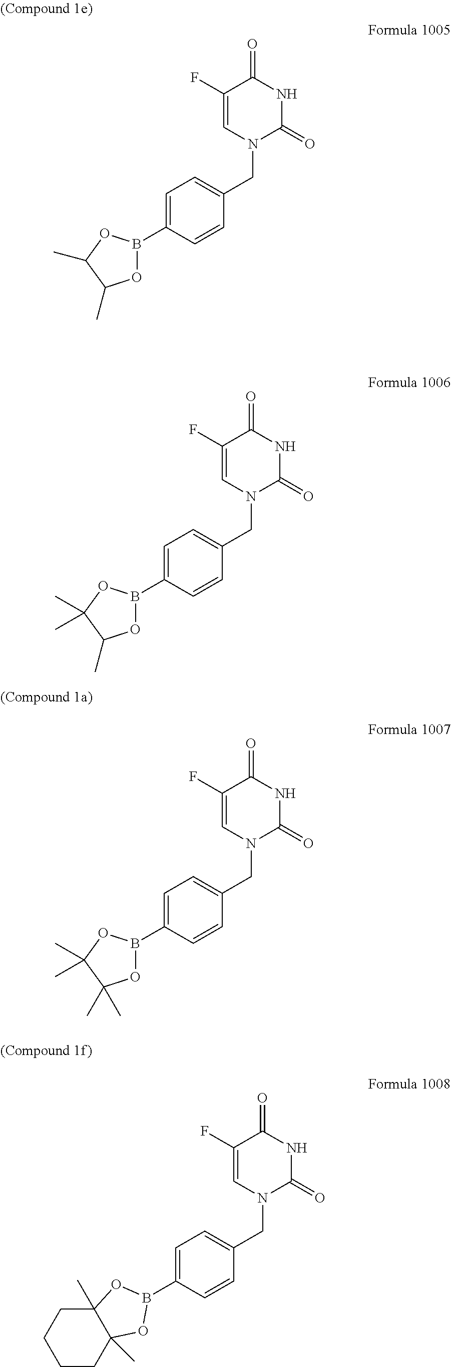 [SMILES: CC1OB(c2ccc(Cn3cc(F)c(=O)[nH]c3=O)cc2)OC1C ; CC1OB(c2ccc(Cn3cc(F)c(=O)[nH]c3=O)cc2)OC1(C)C ; CC1(C)OB(c2ccc(Cn3cc(F)c(=O)[nH]c3=O)cc2)OC1(C)C ; CC12CCCCC1(C)OB(c1ccc(Cn3cc(F)c(=O)[nH]c3=O)cc1)O2]

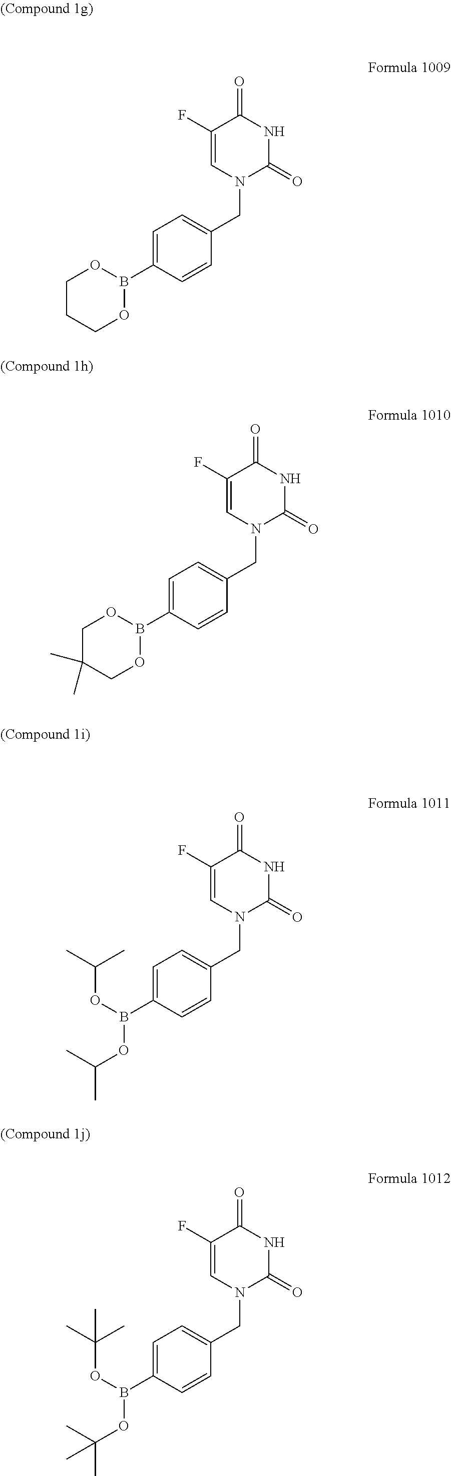 [SMILES: O=c1[nH]c(=O)n(Cc2ccc(B3OCCCO3)cc2)cc1F ; CC1(C)COB(c2ccc(Cn3cc(F)c(=O)[nH]c3=O)cc2)OC1 ; CC(C)OB(OC(C)C)c1ccc(Cn2cc(F)c(=O)[nH]c2=O)cc1 ; CC(C)(C)OB(OC(C)(C)C)c1ccc(Cn2cc(F)c(=O)[nH]c2=O)cc1]

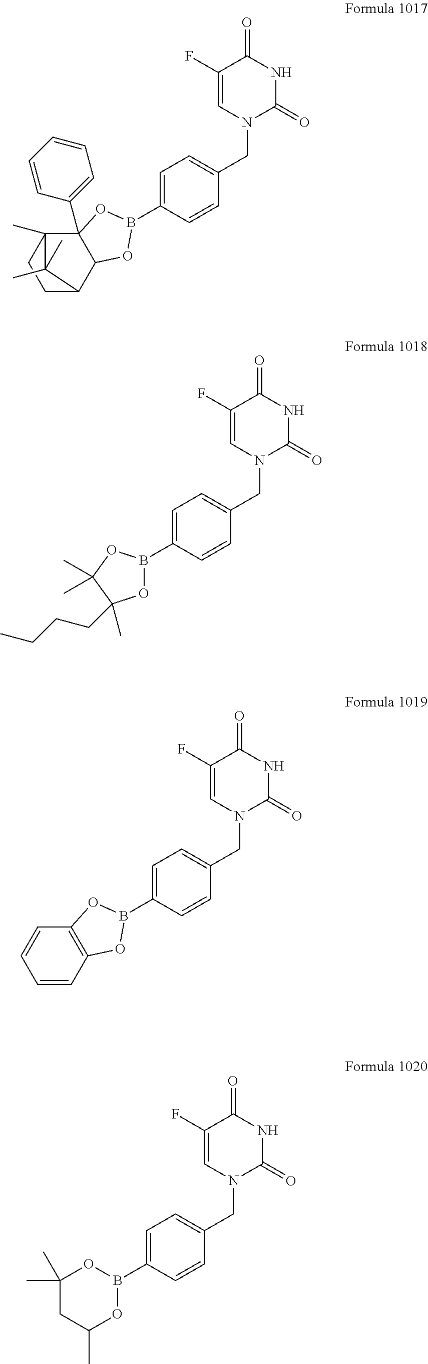 [SMILES: CC1(C)C2CCC1(C)C1(c3ccccc3)OB(c3ccc(Cn4cc(F)c(=O)[nH]c4=O)cc3)OC21 ; CCCCC1(C)OB(c2ccc(Cn3cc(F)c(=O)[nH]c3=O)cc2)OC1(C)C ; O=c1[nH]c(=O)n(Cc2ccc(B3Oc4ccccc4O3)cc2)cc1F ; CC1CC(C)(C)OB(c2ccc(Cn3cc(F)c(=O)[nH]c3=O)cc2)O1]

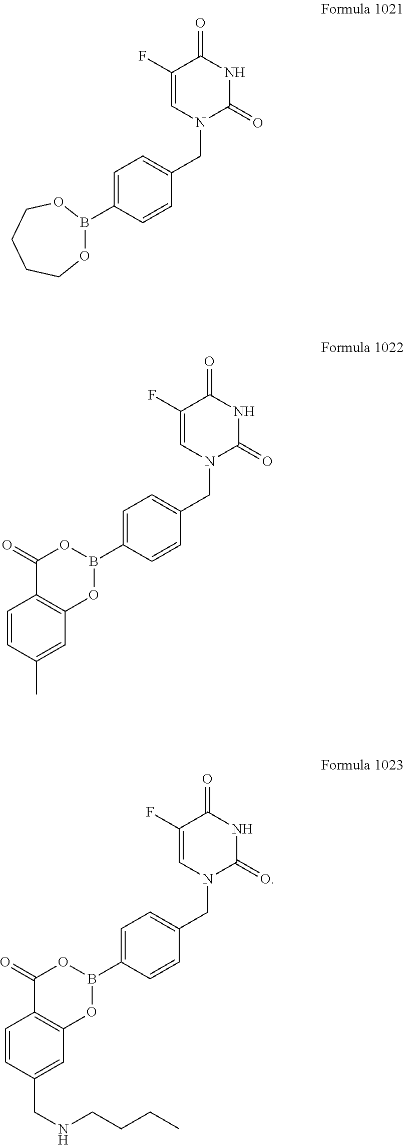 [SMILES: O=c1[nH]c(=O)n(Cc2ccc(B3OCCCCO3)cc2)cc1F ; Cc1ccc2c(c1)OB(c1ccc(Cn3cc(F)c(=O)[nH]c3=O)cc1)OC2=O ; CCCCNCc1ccc2c(c1)OB(c1ccc(Cn3cc(F)c(=O)[nH]c3=O)cc1)OC2=O]

In some embodiments, the invention relates to a compound of any one of Formulas 2001-2003:

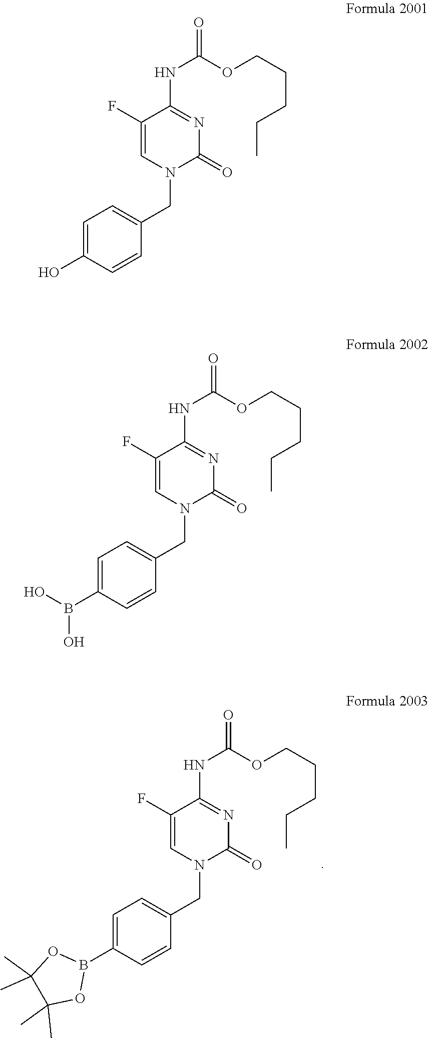 [SMILES: CCCCCOC(=O)Nc1nc(=O)n(Cc2ccc(O)cc2)cc1F ; CCCCCOC(=O)Nc1nc(=O)n(Cc2ccc(B(O)O)cc2)cc1F ; CCCCCOC(=O)Nc1nc(=O)n(Cc2ccc(B3OC(C)(C)C(C)(C)O3)cc2)cc1F]

In some embodiments, the disclosure provides compounds any one of Formulas 1001-a to 1023-a:

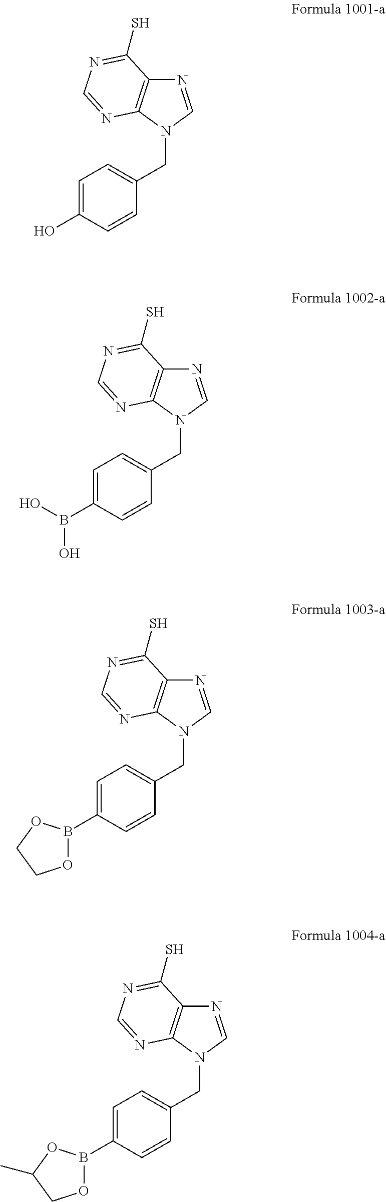 [SMILES: Oc1ccc(Cn2cnc3c(S)ncnc32)cc1 ; OB(O)c1ccc(Cn2cnc3c(S)ncnc32)cc1 ; Sc1ncnc2c1ncn2Cc1ccc(B2OCCO2)cc1 ; CC1COB(c2ccc(Cn3cnc4c(S)ncnc43)cc2)O1]

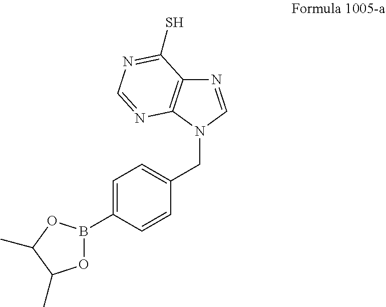 [SMILES: CC1OB(c2ccc(Cn3cnc4c(S)ncnc43)cc2)OC1C]

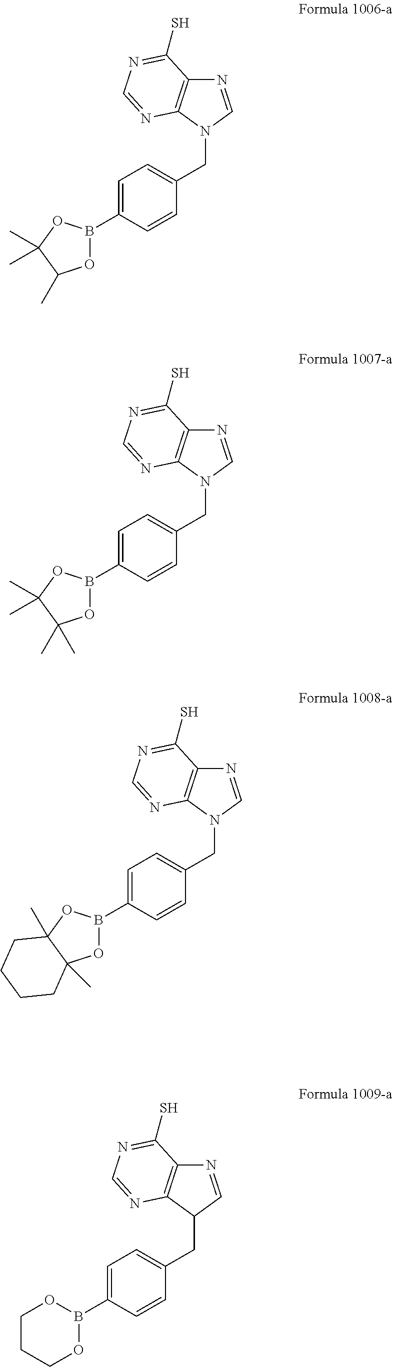 [SMILES: CC1OB(c2ccc(Cn3cnc4c(S)ncnc43)cc2)OC1(C)C ; CC1(C)OB(c2ccc(Cn3cnc4c(S)ncnc43)cc2)OC1(C)C ; CC12CCCCC1(C)OB(c1ccc(Cn3cnc4c(S)ncnc43)cc1)O2 ; Sc1ncnc2c1N=CC2Cc1ccc(B2OCCCO2)cc1]

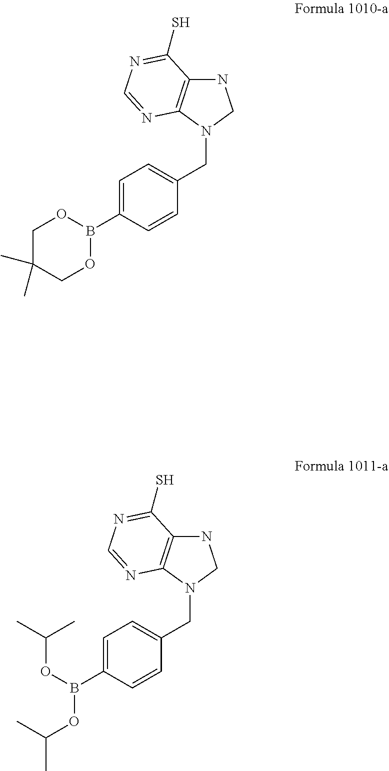 [SMILES: CC1(C)COB(c2ccc(CN3CNc4c(S)ncnc43)cc2)OC1 ; CC(C)OB(OC(C)C)c1ccc(CN2CNc3c(S)ncnc32)cc1]

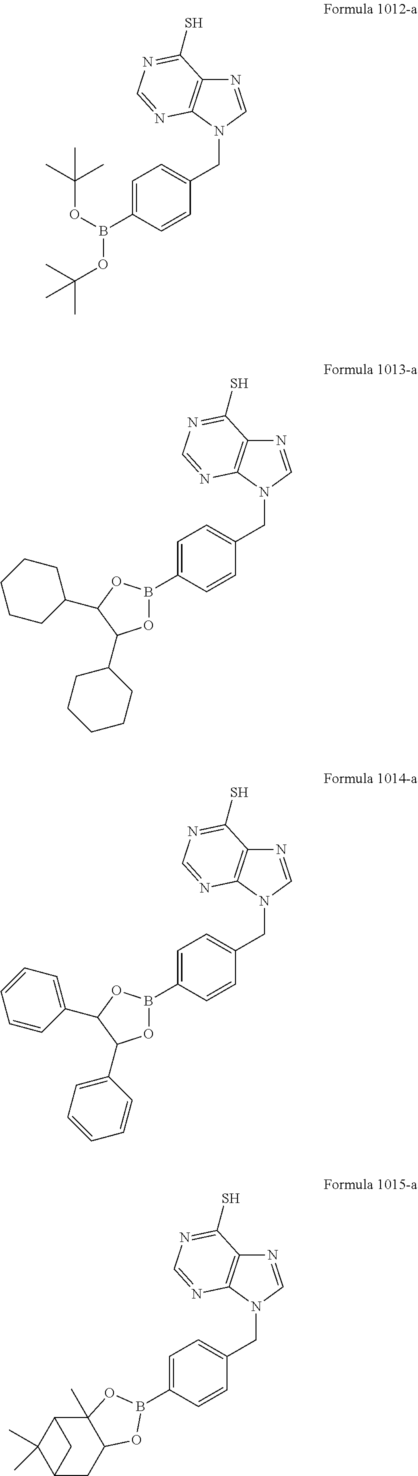 [SMILES: CC(C)(C)OB(OC(C)(C)C)c1ccc(Cn2cnc3c(S)ncnc32)cc1 ; Sc1ncnc2c1ncn2Cc1ccc(B2OC(C3CCCCC3)C(C3CCCCC3)O2)cc1 ; Sc1ncnc2c1ncn2Cc1ccc(B2OC(c3ccccc3)C(c3ccccc3)O2)cc1 ; CC1(C)C2CC3OB(c4ccc(Cn5cnc6c(S)ncnc65)cc4)OC3(C)C1C2]

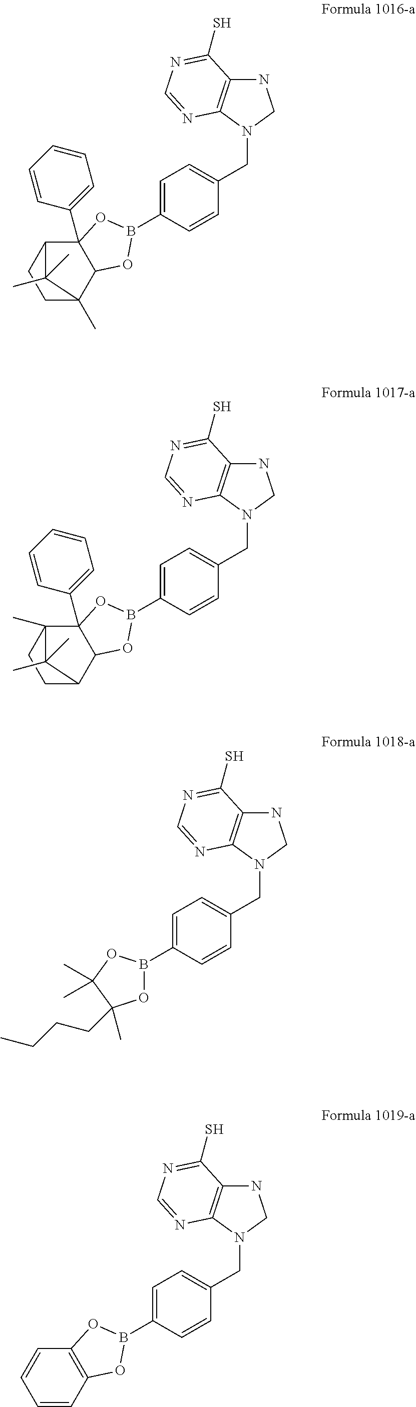 [SMILES: CC1(C)C2CCC1(C)C1OB(c3ccc(CN4CNc5c(S)ncnc54)cc3)OC21c1ccccc1 ; CC1(C)C2CCC1(C)C1(c3ccccc3)OB(c3ccc(CN4CNc5c(S)ncnc54)cc3)OC21 ; CCCCC1(C)OB(c2ccc(CN3CNc4c(S)ncnc43)cc2)OC1(C)C ; Sc1ncnc2c1NCN2Cc1ccc(B2Oc3ccccc3O2)cc1]

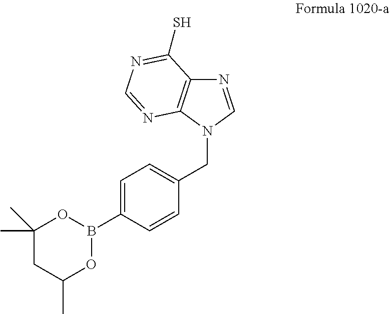 [SMILES: CC1CC(C)(C)OB(c2ccc(Cn3cnc4c(S)ncnc43)cc2)O1]

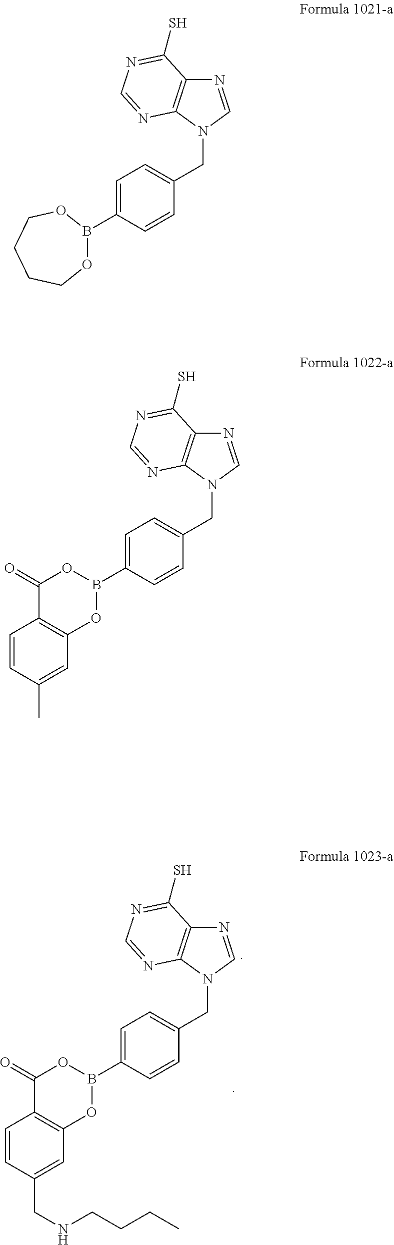 [SMILES: Sc1ncnc2c1ncn2Cc1ccc(B2OCCCCO2)cc1 ; Cc1ccc2c(c1)OB(c1ccc(Cn3cnc4c(S)ncnc43)cc1)OC2=O ; CCCCNCc1ccc2c(c1)OB(c1ccc(Cn3cnc4c(S)ncnc43)cc1)OC2=O]

In some embodiments, the disclosure provides compounds of any one of Formulas 1001-b to 1023-b:

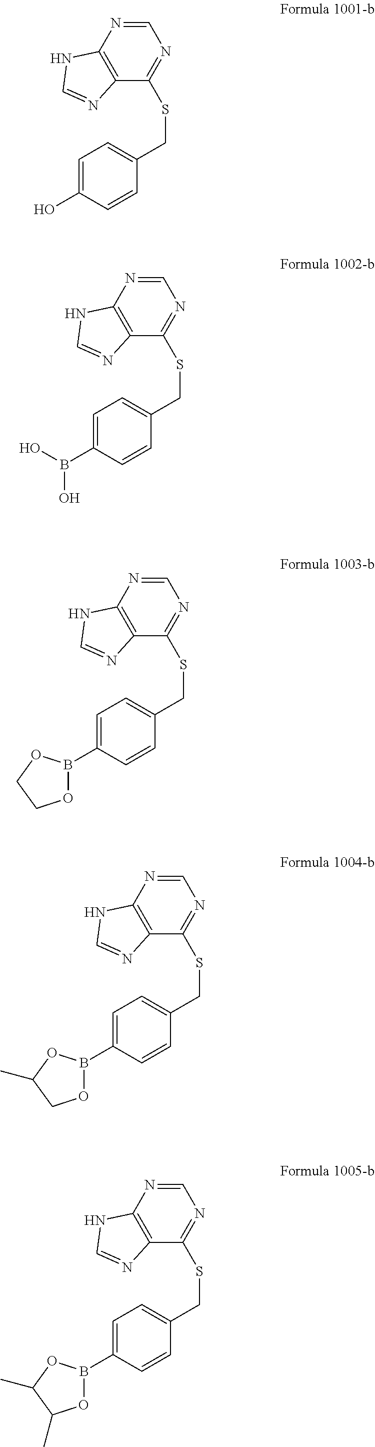 [SMILES: Oc1ccc(CSc2ncnc3[nH]cnc23)cc1 ; OB(O)c1ccc(CSc2ncnc3[nH]cnc23)cc1 ; c1nc(SCc2ccc(B3OCCO3)cc2)c2nc[nH]c2n1 ; CC1COB(c2ccc(CSc3ncnc4[nH]cnc34)cc2)O1 ; CC1OB(c2ccc(CSc3ncnc4[nH]cnc34)cc2)OC1C]

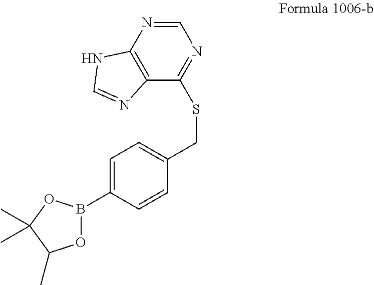 [SMILES: CC1OB(c2ccc(CSc3ncnc4[nH]cnc34)cc2)OC1(C)C]

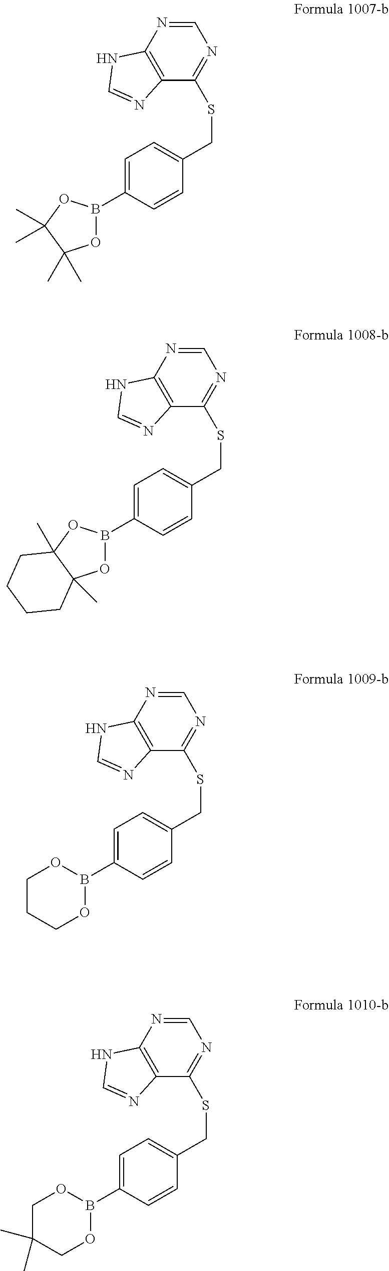 [SMILES: CC1(C)OB(c2ccc(CSc3ncnc4[nH]cnc34)cc2)OC1(C)C ; CC12CCCCC1(C)OB(c1ccc(CSc3ncnc4[nH]cnc34)cc1)O2 ; c1nc(SCc2ccc(B3OCCCO3)cc2)c2nc[nH]c2n1 ; CC1(C)COB(c2ccc(CSc3ncnc4[nH]cnc34)cc2)OC1]

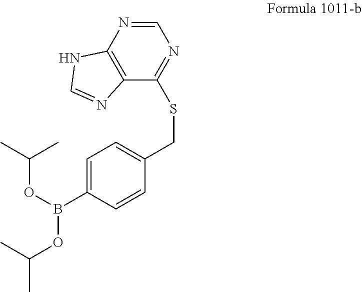 [SMILES: CC(C)OB(OC(C)C)c1ccc(CSc2ncnc3[nH]cnc23)cc1]

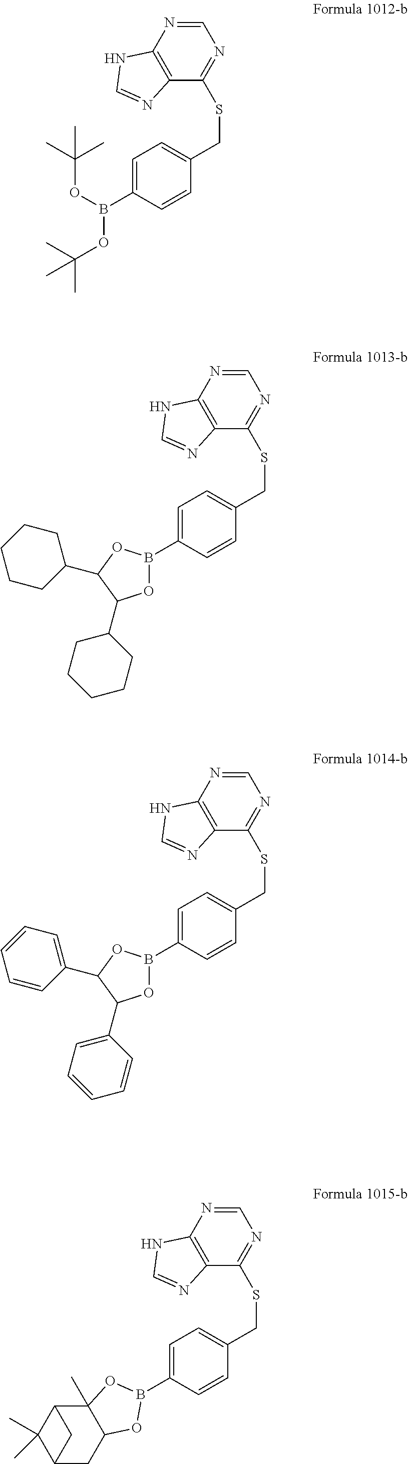 [SMILES: CC(C)(C)OB(OC(C)(C)C)c1ccc(CSc2ncnc3[nH]cnc23)cc1 ; c1nc(SCc2ccc(B3OC(C4CCCCC4)C(C4CCCCC4)O3)cc2)c2nc[nH]c2n1 ; c1ccc(C2OB(c3ccc(CSc4ncnc5[nH]cnc45)cc3)OC2c2ccccc2)cc1 ; CC1(C)C2CC3OB(c4ccc(CSc5ncnc6[nH]cnc56)cc4)OC3(C)C1C2]

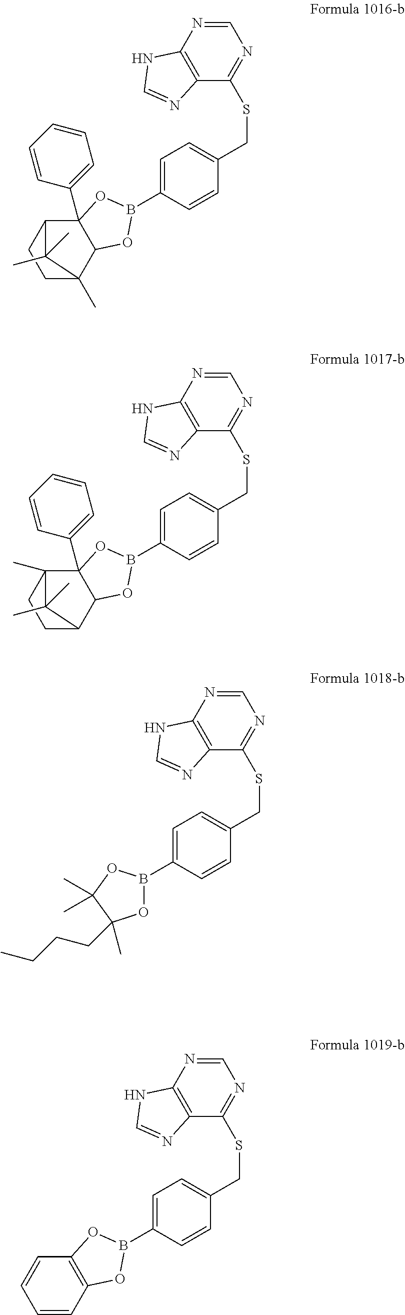 [SMILES: CC1(C)C2CCC1(C)C1OB(c3ccc(CSc4ncnc5[nH]cnc45)cc3)OC21c1ccccc1 ; CC1(C)C2CCC1(C)C1(c3ccccc3)OB(c3ccc(CSc4ncnc5[nH]cnc45)cc3)OC21 ; CCCCC1(C)OB(c2ccc(CSc3ncnc4[nH]cnc34)cc2)OC1(C)C ; c1ccc2c(c1)OB(c1ccc(CSc3ncnc4[nH]cnc34)cc1)O2]

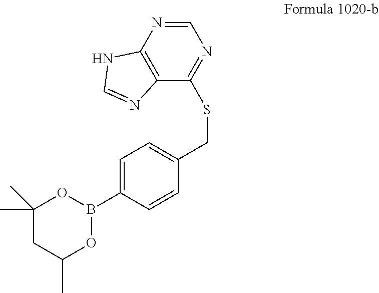 [SMILES: CC1CC(C)(C)OB(c2ccc(CSc3ncnc4[nH]cnc34)cc2)O1]

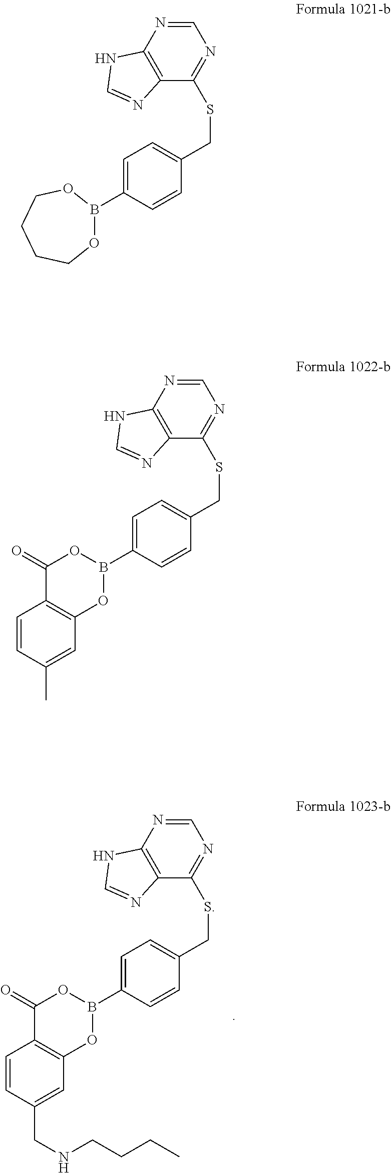 [SMILES: c1nc(SCc2ccc(B3OCCCCO3)cc2)c2nc[nH]c2n1 ; Cc1ccc2c(c1)OB(c1ccc(CSc3ncnc4[nH]cnc34)cc1)OC2=O ; CCCCNCc1ccc2c(c1)OB(c1ccc(CSc3ncnc4[nH]cnc34)cc1)OC2=O]

### Pharmaceutical Compositions

In one embodiment, the disclosure provides a pharmaceutical composition for use in the treatment of the diseases and conditions described herein. In some embodiments, the disclosure provides pharmaceutical compositions, including those described below, for use in the treatment of a hyperproliferative disease. In some embodiments, the disclosure provides pharmaceutical compositions, including those described below, for use in the treatment of cancer.

In some embodiments, the disclosure provides a pharmaceutical composition including one or more compounds of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, or a pharmaceutically acceptable salt, solvate, hydrate, cocrystal, or prodrug thereof, and a pharmaceutically acceptable carrier.

In some embodiments, the disclosure provides a pharmaceutical composition including one or more compounds of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, or a pharmaceutically acceptable salt, solvate, hydrate, cocrystal, or prodrug thereof, and a physiologically compatible carrier medium.

In some embodiments, the disclosure provides pharmaceutical compositions for treating a disease alleviated by administration of 5-FU. In some embodiments, the disclosure provides pharmaceutical compositions for treating a disease alleviated by administration of 6-MP. The pharmaceutical compositions are typically formulated to provide a therapeutically effective amount of one or more compound of any one of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, or a fragment, derivative, conjugate, variant, radioisotope-labeled complex, or biosimilar thereof, or pharmaceutically acceptable salts, prodrugs, solvates, or hydrates thereof, as the active ingredients. Where desired, the pharmaceutical compositions contain a pharmaceutically acceptable salt and/or coordination complex of one or more of the active ingredients. Typically, the pharmaceutical compositions also comprise one or more pharmaceutically acceptable excipients, carriers, including inert solid diluents and fillers, diluents, including sterile aqueous solution and various organic solvents, permeation enhancers, solubilizers and adjuvants.

In some embodiments, the pharmaceutical compositions described above are for use in the treatment of the diseases and conditions described herein. In some embodiments, the pharmaceutical compositions are for use in the treatment of cancer. In one embodiment, the pharmaceutical compositions of the present disclosure are for use in the treatment of a cancer selected from the group consisting of bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thymoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, ovarian cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophageal tumors, hematological neoplasms, non-small-cell lung cancer, chronic myelocytic leukemia, diffuse large B-cell lymphoma, esophagus tumor, follicle center lymphoma, head and neck tumor, hepatitis C virus related cancer, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma. In some embodiments, the diseases is a disease alleviated by 5-fluorouracil (5-FU) chemotherapy. In some embodiments, the diseases is a disease alleviated by 6-mercaptopurine (6-MP) chemotherapy. In some embodiments, any side effects of the treatment are reduced compared to the side effects of a treatment including administering to the patient a therapeutically equivalent amount of 5-fluorouracil (5-FU) or 6-mercaptopurine (6-MP).

In some embodiments, the disclosure provides methods of treating a disease alleviated by anti-cancer or anti-autoimmune diseases chemotherapy in a patient in need thereof, comprising administering to the patient a compound or a pharmaceutical composition thereof described herein, wherein the disease is an autoimmune disease. In some embodiments, the autoimmune disease is selected from achalasia, Addison's disease, adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticarial, axonal and neuronal neuropathy (AMAN), Baló disease, Behcet's disease, benign mucosal pemphigoid, bullous pemphigoid, Castleman disease (CD), celiac disease, Chagas disease, chronic inflammatory demyelinating polyneuropathy (CIDP), chronic recurrent multifocal osteomyelitis (CRMO), Churg-Strauss syndrome (CSS), eosinophilic granulomatosis (EGPA), cicatricial pemphigoid, Cogan's syndrome, cold agglutinin disease, congenital heart block, Coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), herpes gestationis, pemphigoid gestationis (PG), hidradenitis suppurativa (HS; acne inversa), hypogammalglobulinemia, IgA nephropathy, IgG4-related sclerosing disease, immune thrombocytopenic purpura (ITP), inclusion body myositis (IBM), interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, Lyme disease chronic, Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, Pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes type I, II, III, polymyalgia rheumatic, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm and testicular autoimmunity, stiff person syndrome (SPS), subacute bacterial endocarditis (SBE), Susac's syndrome, sympathetic ophthalmia (SO), Takayasu's arteritis, temporal arteritis, giant cell arteritis, thrombocytopenic purpura (TTP), Tolosa-Hunt syndrome (THS), transverse myelitis, type 1 diabetes, ulcerative colitis (UC), undifferentiated connective tissue disease (UCTD), uveitis, vasculitis, vitiligo, and Vogt-Koyanagi-Harada Disease. In some embodiments, the diseases is a disease alleviated by 5-fluorouracil (5-FU) chemotherapy. In some embodiments, the diseases is a disease alleviated by 6-mercaptopurine (6-MP) chemotherapy. In some embodiments, any side effects of the treatment are reduced compared to the side effects of a treatment including administering to the patient a therapeutically equivalent amount of 5-fluorouracil (5-FU) or 6-mercaptopurine (6-MP).

Where desired, other active pharmaceutical ingredient(s) may be mixed into a preparation or two or more components of the combination may be formulated into separate preparations for use in combination separately or at the same time. A kit containing the components of the combination, formulated into separate preparations for said use, is also provided by the disclosure.

In some embodiments, the concentration of any one compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, provided in a pharmaceutical composition of the disclosure, is independently less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.00010% w/w, w/v, or v/v of the pharmaceutical composition.

In some embodiments, the concentration of any one compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, provided in a pharmaceutical composition of the disclosure, is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v of the pharmaceutical composition.

In some embodiments, the concentration of any one compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, provided in a pharmaceutical composition of the disclosure, is independently in the range from about 0.0001% to about 50%, from about 0.001% to about 40%, from about 0.01% to about 30%, from about 0.02% to about 29%, from about 0.03% to about 28%, from about 0.04% to about 27%, from about 0.05% to about 26%, from about 0.06% to about 25%, from about 0.07% to about 24%, from about 0.08% to about 23%, from about 0.09% to about 22%, from about 0.1% to about 21%, from about 0.2% to about 20%, from about 0.3% to about 19%, from about 0.4% to about 18%, from about 0.5% to about 17%, from about 0.6% to about 16%, from about 0.7% to about 15%, from about 0.8% to about 14%, from about 0.9% to about 12%, or from about 1% to about 10% w/w, w/v, or v/v of the pharmaceutical composition.

In some embodiments, the concentration of any one compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, provided in a pharmaceutical composition of the disclosure, is independently in the range from about 0.001% to about 10%, from about 0.01% to about 5%, from about 0.02% to about 4.5%, from about 0.03% to about 4%, from about 0.04% to about 3.5%, from about 0.05% to about 3%, from about 0.06% to about 2.5%, from about 0.07% to about 2%, from about 0.08% to about 1.5%, from about 0.09% to about 10%, from about 0.10% to about 0.9% w/w, w/v, or v/v of the pharmaceutical composition.

In some embodiments, the concentration of any one compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, provided in a pharmaceutical composition of the disclosure, is independently equal to or less than about 10 g, about 9.5 g, about 9.0 g, about 8.5 g, about 8.0 g, about 7.5 g, about 7.0 g, about 6.5 g, about 6.0 g, about 5.5 g, about 5.0 g, about 4.5 g, about 4.0 g, about 3.5 g, about 3.0 g, about 2.5 g, about 2.0 g, about 1.5 g, about 1.0 g, about 0.95 g, about 0.9 g, about 0.85 g, about 0.8 g, about 0.75 g, about 0.7 g, about 0.65 g, about 0.6 g, about 0.55 g, about 0.5 g, about 0.45 g, about 0.4 g, about 0.35 g, about 0.3 g, about 0.25 g, about 0.2 g, about 0.15 g, about 0.1 g, about 0.09 g, about 0.08 g, about 0.07 g, about 0.06 g, about 0.05 g, about 0.04 g, about 0.03 g, about 0.02 g, about 0.01 g, about 0.009 g, about 0.008 g, about 0.007 g, about 0.006 g, about 0.005 g, about 0.004 g, about 0.003 g, about 0.002 g, about 0.001 g, about 0.0009 g, about 0.0008 g, about 0.0007 g, about 0.0006 g, about 0.0005 g, about 0.0004 g, about 0.0003 g, about 0.0002 g, or about 0.0001 g.

In some embodiments, the concentration of any one compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, provided in a pharmaceutical composition of the disclosure, is independently more than about 0.0001 g, about 0.0002 g, about 0.0003 g, about 0.0004 g, about 0.0005 g, about 0.0006 g, about 0.0007 g, about 0.0008 g, about 0.0009 g, about 0.001 g, about 0.0015 g, about 0.002 g, about 0.0025 g, about 0.003 g, about 0.0035 g, about 0.004 g, about 0.0045 g, about 0.005 g, about 0.0055 g, about 0.006 g, about 0.0065 g, about 0.007 g, about 0.0075 g, about 0.008 g, about 0.0085 g, about 0.009 g, about 0.0095 g, about 0.01 g, about 0.015 g, about 0.02 g, about 0.025 g, about 0.03 g, about 0.035 g, about 0.04 g, about 0.045 g, about 0.05 g, about 0.055 g, about 0.06 g, about 0.065 g, about 0.07 g, about 0.075 g, about 0.08 g, about 0.085 g, about 0.09 g, about 0.095 g, about 0.1 g, about 0.15 g, about 0.2 g, about 0.25 g, about 0.3 g, about 0.35 g, about 0.4 g, about 0.45 g, about 0.5 g, about 0.55 g, about 0.6 g, about 0.65 g, about 0.7 g, about 0.75 g, about 0.8 g, about 0.85 g, about 0.9 g, about 0.95 g, about 1 g, about 1.5 g, about 2 g, about 2.5, about 3 g, about 3.5, about 4 g, about 4.5 g, about 5 g, about 5.5 g, about 6 g, about 6.5 g, about 7 g, about 7.5 g, about 8 g, about 8.5 g, about 9 g, about 9.5 g, or about 10 g.

Each of the compounds of any of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, according to the disclosure, is effective over a wide dosage range. For example, in the treatment of adult humans, dosages independently ranging from about 0.01 to about 1000 mg, from about 0.5 to about 100 mg, from about 1 to about 50 mg per day, and from about 5 to about 40 mg per day are examples of dosages that may be used. The exact dosage will depend upon the route of administration, the form in which the compound is administered, the gender and age of the subject to be treated, the body weight of the subject to be treated, and the preference and experience of the attending physician.

Described below are non-limiting pharmaceutical compositions and methods for preparing the same.

### Pharmaceutical Compositions for Oral Administration

In preferred embodiments, the disclosure provides a pharmaceutical composition for oral administration containing one or more of a compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, according to the disclosure, and a pharmaceutical excipient suitable for administration.

In other embodiments, the disclosure provides a pharmaceutical composition for oral administration containing one or more of a compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, according to the disclosure, a pharmaceutical excipient suitable for administration, and one or more additional active pharmaceutical ingredients.

In some embodiments, the pharmaceutical composition may be a solid pharmaceutical composition suitable for oral consumption. In some embodiments, the pharmaceutical composition may be a liquid pharmaceutical composition suitable for oral consumption.

Pharmaceutical compositions of the invention suitable for oral administration can be presented as discrete dosage forms, such as capsules, sachets, tablets, liquids, or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients.

Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The disclosure further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained.

Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Each of the compounds of any of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, according to the disclosure, used as active ingredients, can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof, lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl camitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present disclosure and to minimize precipitation of the compound of the present disclosure. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of about 10%, about 25%, about 50%, about 100%, or up to about 200% by weight, based on the combined weight of the drug, and other excipients. If desired, very small amounts of solubilizer may also be used, such as about 5%, about 2%, about 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not be limited to, sodium, potassium, lithium, magnesium, calcium, and/or ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In some embodiments, the disclosure provides a pharmaceutical composition for injection containing a compound of any of Formulas I-IV, 101-110, 1001-1024, and 2001-2003, according to the disclosure, and a pharmaceutical excipient suitable for injection. Components and amounts of agents in the compositions are as described herein.

The forms in which the compositions of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, and thimerosal.

Sterile injectable solutions are prepared by incorporating a compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, according to the disclosure, in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical Delivery

In some embodiments, the disclosure provides a pharmaceutical composition for transdermal delivery containing a compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, according to the disclosure, and a pharmaceutical excipient suitable for transdermal delivery.

Compositions of the present disclosure can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present disclosure employs transdermal delivery devices (“patches”). Such transdermal patches may be used to provide continuous or discontinuous infusion of the compounds of the disclosure, either with or without another active pharmaceutical ingredient.

The construction and use of transdermal patches for the delivery of pharmaceutical agents is known in the art. See, e.g., U.S. Pat. Nos. 5,023,252; 4,992,445 and 5,001,139. Such patches may be constructed for continuous, pulsatile, or on demand delivery of pharmaceutical agents.

### Pharmaceutical Compositions for Inhalation

In some embodiments, the disclosure provides a pharmaceutical composition for inhalation or insufflation delivery containing a compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, according to the disclosure. Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. Dry powder inhalers may also be used to provide inhaled delivery of the compositions.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, et al., eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of any one compound of any of Formulas I-IV, 101-110, 1001-1024, and 2001-2003, according to the disclosure, or pharmaceutical compositions of these compounds, can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. The compounds or compositions thereof can also be administered intraadiposally or intrathecally.

Exemplary parenteral administration forms include solutions or suspensions of active compound in sterile aqueous solutions, for example, aqueous propylene glycol or dextrose solutions. Such dosage forms can be suitably buffered, if desired.

The disclosure also provides kits. The kits include any one compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, according to the disclosure, or pharmaceutical compositions thereof, either alone or in combination in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient.

Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

In some embodiments, the disclosure provides a kit including a composition including a therapeutically effective amount of a compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, according to the disclosure, or a fragment, derivative, conjugate, variant, radioisotope-labeled complex, biosimilar, pharmaceutically acceptable salt, solvate, hydrate, cocrystal, or prodrug thereof. These compositions are typically pharmaceutical compositions.

In some embodiments, the kits described herein are for use in the treatment of the diseases and conditions described herein. In some embodiments, the kits are for use in the treatment of cancer. In some embodiments, the kits are for use in treating solid tumor cancers, lymphomas and leukemias.

In some embodiments, the kits of the present disclosure are for use in the treatment of a cancer selected from the group consisting of bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thymoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, ovarian cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophageal tumors, hematological neoplasms, non-small-cell lung cancer, chronic myelocytic leukemia, diffuse large B-cell lymphoma, esophagus tumor, follicle center lymphoma, head and neck tumor, hepatitis C virus related cancer, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma.

In some embodiments, the kits of the present disclosure are for use in the treatment of a cancer selected from malignant pancreatic insulinoma, malignant carcinoid carcinoma, mycosis fungoides, malignant hypercalcemia, cervical hyperplasia, leukemia, myeloid leukemia, acute myeloid leukemia, acute lymphocytic leukemia, chronic lymphocytic leukemia, acute myelogenous leukemia, chronic myelogenous leukemia, chronic granulocytic leukemia, chronic myeloid leukemia, acute granulocytic leukemia, hairy cell leukemia, acute erythroleukemic leukemia, acute lymphoblastic B-cell leukemia, acute lymphoblastic T-cell leukemia, acute lymphoblastic leukemia, acute megakaryoblastic leukemia, acute monoblastic leukemia, acute myeloblastic leukemia, acute myelomonocytic leukemia, acute nonlymphocytic leukemia, acute promyelocytic leukemia, acute undifferentiated leukemia, chronic lymphocytic leukemia, chronic myelocytic leukemia, hairy cell leukemia, rhabdomyosarcoma, Kaposi's sarcoma, essential thrombocytosis, soft-tissue sarcoma, retinoblastoma, acoustic neuroma, adenocarcinoma, angiosarcoma, adrenal carcinoma, adrenal cortex carcinoma, astrocytoma, basal cell carcinoma, bile duct carcinoma, bladder cancer, bladder carcinoma, brain cancer, breast cancer, breast carcinoma, bronchogenic carcinoma, cervical cancer, cervical carcinoma, chordoma, choriocarcinoma, colon cancer, colon carcinoma, colorectal cancer, craniopharyngioma, cystadenocarcinoma, embryonal carcinoma, endotheliocarcinoma, endometrial carcinoma, ependymoma, epithelial carcinoma, esophageal cancer, esophageal carcinoma, Ewing's tumor, fibrosarcoma, gastric cancer, genitourinary carcinoma, glioblastoma, glioblastoma multiforme, glioma, head and neck cancer, hemangioblastoma, hepatoma, kidney cancer, leiomyosarcoma, liposarcoma, liver cancer, lung cancer, lung carcinoma, lymphangioendotheliosarcoma, lymphangiosarcoma, lymphoma, medullary carcinoma, medulloblastoma, malignant melanoma, melanoma, meningioma, mesothelioma, myxosarcoma, myeloma, nasal cancer, neuroblastoma, non-small cell lung cancer, oligodendroglioma, oral cancer, osteogenic sarcoma, ovarian cancer, ovarian carcinoma, pancreatic cancer, pancreatic carcinoma, papillary adenocarcinoma, papillary carcinoma, pinealoma, prostate cancer, prostatic carcinoma, primary brain carcinoma, rabdomyosarcoma, rectal cancer, renal cell carcinoma, retinoblastoma, sarcoma, sebaceous gland carcinoma, seminoma, skin cancer, squamous cell carcinoma, stomach cancer, stomach carcinoma, sweat gland carcinoma, synovioma, testicular cancer, testicular carcinoma, thyroid carcinoma, small cell lung carcinoma, throat cancer, uterine cancer, Wilm's tumor, blood cancer, heavy chain disease, Hodgkin's disease, multiple myeloma, non-Hodgkin's lymphoma, polycythemia vera, primary macroglobulinemia, and Waldenstrom's macroglobulinemia.

In some embodiments, the kits of the present disclosure are for use in the treatment of an autoimmune disease selected from achalasia, Addison's disease, adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticarial, axonal and neuronal neuropathy (AMAN), Baló disease, Behcet's disease, benign mucosal pemphigoid, bullous pemphigoid, Castleman disease (CD), celiac disease, Chagas disease, chronic inflammatory demyelinating polyneuropathy (CIDP), chronic recurrent multifocal osteomyelitis (CRMO), Churg-Strauss syndrome (CSS), eosinophilic granulomatosis (EGPA), cicatricial pemphigoid, Cogan's syndrome, cold agglutinin disease, congenital heart block, Coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), herpes gestationis, pemphigoid gestationis (PG), hidradenitis suppurativa (HS; acne inversa), hypogammalglobulinemia, IgA nephropathy, IgG4-related sclerosing disease, immune thrombocytopenic purpura (ITP), inclusion body myositis (IBM), interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, Lyme disease chronic, Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, Pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes type I, II, III, polymyalgia rheumatic, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm and testicular autoimmunity, stiff person syndrome (SPS), subacute bacterial endocarditis (SBE), Susac's syndrome, sympathetic ophthalmia (SO), Takayasu's arteritis, temporal arteritis, giant cell arteritis, thrombocytopenic purpura (TTP), Tolosa-Hunt syndrome (THS), transverse myelitis, type 1 diabetes, ulcerative colitis (UC), undifferentiated connective tissue disease (UCTD), uveitis, vasculitis, vitiligo, and Vogt-Koyanagi-Harada Disease.

### Dosages and Dosing Regimens

The amounts to be administered of any one compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, will be dependent on the human or mammal being treated, the severity of the disorder or condition, the rate of administration, the disposition of the compounds and the discretion of the prescribing physician. However, an effective dosage of each is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect, e.g., by dividing such larger doses into several small doses for administration throughout the day. The dosage of any one compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, may be provided in units of mg/kg of body mass, or in mg/m² of body surface area.

In some embodiments, the compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, is administered in a single dose. Such administration may be by injection, e.g., intravenous injection, in order to introduce the compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, quickly. However, other routes, including the oral route, may be used as appropriate. A single dose of any one compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, may also be used for treatment of an acute condition.

In some embodiments, the compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, is administered in multiple doses. In some embodiments, the compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, is administered in multiple doses. Dosing may be once, twice, three times, four times, five times, six times, or more than six times per day. Dosing may be once a month, once every two weeks, once a week, or once every other day. In other embodiments, the compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, is administered about once per day to about 6 times per day. In some embodiments, the compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, is administered once daily, while in other embodiments, the compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, is administered twice daily, and in other embodiments the compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, is administered three times daily.

Administration of the active pharmaceutical ingredients of the disclosure may continue as long as necessary. In some embodiments, the compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, is administered for more than 1, 2, 3, 4, 5, 6, 7, 14, or 28 days. In some embodiments, the compound Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, is administered for less than 28, 14, 7, 6, 5, 4, 3, 2, or 1 day. In some embodiments, the compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, is administered chronically on an ongoing basis, e.g., for the treatment of chronic effects. In another embodiment the administration of the compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, continues for less than about 7 days. In yet another embodiment the administration continues for more than about 6, 10, 14, 28 days, two months, six months, or one year. In some cases, continuous dosing is achieved and maintained as long as necessary.

In some embodiments, an effective dosage of a compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, disclosed herein, is in the range of about 1 mg to about 50 mg, about 5 mg to about 45 mg, about 10 mg to about 40 mg, about 15 mg to about 35 mg, about 20 mg to about 30 mg, about 23 mg to about 28 mg, about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, about 198 to about 202 mg, or about 198 to about 207 mg.

In some embodiments, an effective dosage of a compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, disclosed herein, is about 25 mg, about 50 mg, about 75 mg, about 100 mg, about 125 mg, about 150 mg, about 175 mg, about 200 mg, about 225 mg, or about 250 mg.

In some embodiments, an effective dosage of a compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, disclosed herein, is in the range of about 0.01 mg/kg to about 0.7 mg/kg, about 0.07 mg/kg to about 0.65 mg/kg, about 0.15 mg/kg to about 0.6 mg/kg, about 0.2 mg/kg to about 0.5 mg/kg, about 0.3 mg/kg to about 0.45 mg/kg, about 0.3 mg/kg to about 0.4 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg to about 1.6 mg/kg, about 1.35 mg/kg to about 1.5 mg/kg, about 1.4 mg/kg to about 1.45 mg/kg, about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some embodiments, an effective dosage of a compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, disclosed herein, is about 0.35 mg/kg, about 0.4 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg.

In some instances, dosage levels below the lower limit of the aforesaid ranges may be more than adequate, while in other cases still larger doses may be employed without causing any harmful side effect—e.g., by dividing such larger doses into several small doses for administration throughout the day.

An effective amount of the combination of a compound of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

### Methods of Treating Solid Tumor Cancers, Hematological Malignancies, Inflammation, Immune and Autoimmune Disorders, and Other Diseases

In some embodiments, the disclosure relates to a method of treating a disease alleviated by 5-fluorouracil (5-FU) chemotherapy in a patient in need thereof, including administering to the patient a therapeutically effective amount of one or more compounds of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, or a pharmaceutically acceptable salt, solvate, hydrate, cocrystal, or prodrug thereof. In some embodiments, the disclosure relates to a method of treating a disease alleviated by 6-mercaptopurine (6-MP) chemotherapy in a patient in need thereof, including administering to the patient a therapeutically effective amount of one or more compounds of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, or a pharmaceutically acceptable salt, solvate, hydrate, cocrystal, or prodrug thereof. In some embodiments, the disclosure relates to a method of treating a disease alleviated by chemotherapy with a compound of any of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, in a patient in need thereof, including administering to the patient a therapeutically effective amount of one or more compounds of any of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, or a pharmaceutically acceptable salt, solvate, hydrate, cocrystal, or prodrug thereof.

In some embodiments, the disclosure relates to a method of treating a disease alleviated by 5-fluorouracil (5-FU) or 6-MP chemotherapy in a patient in need thereof, including administering to the patient a dosage unit form including a therapeutically effective amount of one or more compounds of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, or a pharmaceutically acceptable salt, solvate, hydrate, cocrystal, or prodrug thereof. In some embodiments, the disclosure relates to a method of treating a disease alleviated by chemotherapy with a compounds of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, in a patient in need thereof, including administering to the patient a dosage unit form including a therapeutically effective amount of one or more compounds of Formula I, or any one of Formulas A to D, or any one of Formulas 11 to 25, or any one of Formulas 101 to 123, or any one of Formulas 1001 to 1023, or Formula 2001, Formula 2002, or Formula 2003, or any one of Formulas 1001-a to 1023-a, or any one of Formulas 1001-b to 1023-b, or a pharmaceutically acceptable salt, solvate, hydrate, cocrystal, or prodrug thereof. In some embodiments, the dosage unit form includes a physiologically compatible carrier medium.

In one embodiment, any side effects of the treatment are reduced compared to the side effects of a treatment including administering to the patient a therapeutically equivalent amount of 5-fluorouracil (5-FU). The reduction of side effects can be for up to 100%.

In a preferred embodiment, the patient or subject is a mammal, such as a human. In an embodiment, the patient or subject is a human. In an embodiment, the patient or subject is a companion animal. In an embodiment, the patient or subject is a canine, feline, or equine. In some embodiments, the disease is a hyperproliferative disease or disorder. In some embodiments, the disease is cancer. In some embodiments, the cancer can be bladder cancer, squamous cell carcinoma including head and neck cancer, pancreatic ductal adenocarcinoma (PDA), CNS cancer, pancreatic cancer, colon carcinoma, mammary carcinoma, breast cancer, fibrosarcoma, mesothelioma, renal cell carcinoma, lung carcinoma, thymoma, prostate cancer, colorectal cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, ovarian cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophageal tumors, hematological neoplasms, non-small-cell lung cancer, chronic myelocytic leukemia, diffuse large B-cell lymphoma, esophagus tumor, follicle center lymphoma, head and neck tumor, hepatitis C virus induced cancer, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma. In some embodiments, the diseases is an autoimmune disease selected from achalasia, Addison's disease, adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticarial, axonal and neuronal neuropathy (AMAN), Baló disease, Behcet's disease, benign mucosal pemphigoid, bullous pemphigoid, Castleman disease (CD), celiac disease, Chagas disease, chronic inflammatory demyelinating polyneuropathy (CIDP), chronic recurrent multifocal osteomyelitis (CRMO), Churg-Strauss syndrome (CSS), eosinophilic granulomatosis (EGPA), cicatricial pemphigoid, Cogan's syndrome, cold agglutinin disease, congenital heart block, Coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), herpes gestationis, pemphigoid gestationis (PG), hidradenitis suppurativa (HS; acne inversa), hypogammalglobulinemia, IgA nephropathy, IgG4-related sclerosing disease, immune thrombocytopenic purpura (ITP), inclusion body myositis (IBM), interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, Lyme disease chronic, Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, Pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes type I, II, III, polymyalgia rheumatic, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm and testicular autoimmunity, stiff person syndrome (SPS), subacute bacterial endocarditis (SBE), Susac's syndrome, sympathetic ophthalmia (SO), Takayasu's arteritis, temporal arteritis, giant cell arteritis, thrombocytopenic purpura (TTP), Tolosa-Hunt syndrome (THS), transverse myelitis, type 1 diabetes, ulcerative colitis (UC), undifferentiated connective tissue disease (UCTD), uveitis, vasculitis, vitiligo, and Vogt-Koyanagi-Harada Disease. In some embodiments, the diseases is a disease alleviated by 5-fluorouracil (5-FU) chemotherapy. In some embodiments, the diseases is a disease alleviated by 6-mercaptopurine (6-MP) chemotherapy. In some embodiments, any side effects of the treatment are reduced compared to the side effects of a treatment including administering to the patient a therapeutically equivalent amount of 5-fluorouracil (5-FU) or 6-mercaptopurine (6-MP).

Efficacy of the methods, compounds, and combinations of compounds described herein in treating, preventing and/or managing the indicated diseases or disorders can be tested using various animal models known in the art. Efficacy in treating, preventing and/or managing asthma can be assessed using the ova induced asthma model described, for example, in Lee, et al., J. Allergy Clin. Immunol. 2006, 118, 403-9. Efficacy in treating, preventing and/or managing arthritis (e.g., rheumatoid or psoriatic arthritis) can be assessed using the autoimmune animal models described in, for example, Williams, et al., Chem. Biol. 2010, 17, 123-34, WO 2009/088986, WO 2009/088880, and WO 2011/008302. Efficacy in treating, preventing and/or managing psoriasis can be assessed using transgenic or knockout mouse model with targeted mutations in epidermis, vasculature or immune cells, mouse model resulting from spontaneous mutations, and immuno-deficient mouse model with xenotransplantation of human skin or immune cells, all of which are described, for example, in Boehncke, et al., Clinics in Dermatology, 2007, 25, 596-605. Efficacy in treating, preventing and/or managing fibrosis or fibrotic conditions can be assessed using the unilateral ureteral obstruction model of renal fibrosis, which is described, for example, in Chevalier, et al., Kidney International 2009, 75, 1145-1152; the bleomycin induced model of pulmonary fibrosis described in, for example, Moore, et al., Am. J. Physiol. Lung. Cell. Mol. Physiol. 2008, 294, L152-L160; a variety of liver/biliary fibrosis models described in, for example, Chuang, et al., Clin. Liver Dis. 2008, 12, 333-347 and Omenetti, et al., Laboratory Investigation, 2007, 87, 499-514 (biliary duct-ligated model); or any of a number of myelofibrosis mouse models such as described in Varicchio, et al., Expert Rev. Hematol. 2009, 2(3), 315-334. Efficacy in treating, preventing and/or managing scleroderma can be assessed using a mouse model induced by repeated local injections of bleomycin described, for example, in Yamamoto, et al., J. Invest. Dermatol. 1999, 112, 456-462. Efficacy in treating, preventing and/or managing dermatomyositis can be assessed using a myositis mouse model induced by immunization with rabbit myosin as described, for example, in Phyanagi, et al., Arthritis & Rheumatism, 2009, 60(10), 3118-3127. Efficacy in treating, preventing and/or managing lupus can be assessed using various animal models described, for example, in Ghoreishi, et al., Lupus, 2009, 19, 1029-1035; Ohl, et al., J. Biomed. Biotechnol., 2011, Article ID 432595; Xia, et al., Rheumatology, 2011, 50, 2187-2196; Pau, et al., PLoS ONE, 2012, 7(5), e36761; Mustafa, et al., Toxicology, 2011, 290, 156-168; Ichikawa, et al., Arthritis & Rheumatism, 2012, 62(2), 493-503; Rankin, et al., J. Immunology, 2012, 188, 1656-1667. Efficacy in treating, preventing and/or managing Sjögren's syndrome can be assessed using various mouse models described, for example, in Chiorini, et al., J. Autoimmunity, 2009, 33, 190-196. Models for determining efficacy of treatments for pancreatic cancer are described in Herreros-Villanueva, et al., World J. Gastroenterol. 2012, 18, 1286-1294. Models for determining efficacy of treatments for breast cancer are described, e.g., in Fantozzi, Breast Cancer Res. 2006, 8, 212. Models for determining efficacy of treatments for ovarian cancer are described, e.g., in Mullany, et al., Endocrinology 2012, 153, 1585-92; and Fong, et al., J. Ovarian Res. 2009, 2, 12. Models for determining efficacy of treatments for melanoma are described, e.g., in Damsky, et al., Pigment Cell & Melanoma Res. 2010, 23, 853-859. Models for determining efficacy of treatments for lung cancer are described, e.g., in Meuwissen, et al., Genes & Development, 2005, 19, 643-664. Models for determining efficacy of treatments for lung cancer are described, e.g., in Kim, Clin. Exp. Otorhinolaryngol. 2009, 2, 55-60; and Sano, Head Neck Oncol. 2009, 1, 32. Models for determining efficacy of treatments for colorectal cancer, including the CT26 model, are described in Castle, et al., BMC Genomics, 2013, 15, 190; Endo, et al., Cancer Gene Therapy, 2002, 9, 142-148; Roth et al., Adv. Immunol. 1994, 57, 281-351; Fearon, et al., Cancer Res. 1988, 48, 2975-2980. Efficacy in DLBCL may be assessed using the PiBCL1 murine model and BALB/c (haplotype H-2^{d}) mice. Illidge, et al., Cancer Biother. & Radiopharm. 2000, 15, 571-80. Efficacy in NHL may be assessed using the 38C13 murine model with C3H/HeN (haplotype 2-H^{k}) mice or alternatively the 38C13 Her2/neu model. Timmerman, et al., Blood, 2001, 97, 1370-77; Penichet, et al., Cancer Immunolog. Immunother. 2000, 49, 649-662. Efficacy in CLL may be assessed using the BCL1 model using BALB/c (haplotype H-2d) mice. Dutt, et al., Blood, 2011, 117, 3230-29.

While preferred embodiments of the invention are shown and described herein, such embodiments are provided by way of example only and are not intended to otherwise limit the scope of the invention. Various alternatives to the described embodiments of the invention may be employed in practicing the invention.

### Examples

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

General Information

All reagents were purchased and used without further purification unless otherwise noted. Reactions were monitored using thin-layer chromatography (TLC) on commercial silica-gel plates (GF254). Visualization of the developed plates was performed under UV light (254 nm). Flash column chromatography was performed on silica gel (200-300 mesh). ¹H and ¹³C NMR spectra were recorded on a Varian INOVA 400 MHz NMR spectrometer at 25° C. Chemical shifts (δ) are reported in ppm and referenced to an internal tetramethylsilane standard or to the DMSO-d6 residual peak (δ 2.50) for ¹H NMR. Chemical shifts of ¹³C NMR are reported relative to CDCl₃ (δ 77.0) or DMSO-d6 (δ 39.5). The following abbreviations were used to describe peak-splitting patterns when appropriate: br s, broad singlet; s, singlet; d, doublet; t, triplet; q, quartet; and m, multiplet. Coupling constants, J, were reported in hertz units (Hz). High-resolution mass spectra (HRMS) were obtained on a JEOL AccuTOF with ESI and APCI ion sources and coupled to an Agilent 1100 HPLC system.

5-FU, compounds 2 and 3 were commercially available. Dulbecco Modification of Eagles Medium (DMEM) was purchased from Corning Inc. Fetal bovine serum was purchased from Invitrogen (Carlsbad, Calif.).

All cell lines used in the assays were purchased from ATCC (Manassas, Va.) except MEC1 which was recently characterized. Chemicals were purchased from Sigma Aldrich (St. Louis, Mo.).

Cell Counting Kit-8 (CCK8, Dojingo) was used to carry out the cytotoxicity assessment of the prodrugs. Seeded cells were treated with varying compound concentrations for 48-72 h and the CCK8 kit was used to measure the cytotoxicity. For NAC experiments, this ROS scavenger was pre-incubated with the cells for 2 h and then co-incubated with the prodrugs for additional 48 hours unless otherwise noted.

Western Blot Analysis. Treated cells were washed once with PBS and lysed with RIPA lysis buffer (Santa Cruz Biotechnology, SCBT, #24948) supplemented with sodium orthovanadate, PMSF, protease and phosphatase inhibitor cocktails (Sigma Aldrich). Cell lysates were centrifuge for 15 min at 14,000×g and the supernatant was used for immunoblotting. Protein concentration was normalized for the samples and resolved on a 10-13% SDS-PAGE gel. Following transfer to nitrocellulose membrane, specific mouse primary antibodies from SCBT (PARP1 #sc-8007, caspase 3 #sc-7272) and Cell Signaling (cleaved PARP1 #9541, cleaved caspase-3 #9664) were added and incubated overnight. Secondary antibodies corresponding to the host of the primary antibodies was further incubated with the membrane and washed three times to remove excess antibodies. Odyssey Fc Imaging System (LI-COR, Lincoln, Nebr.) was used to quantify the chemiluminescent of the proteins.

Immunohistochemistry Analysis. MCF7 cells were seeded on poly-D-lysine coated slides. Compounds were incubated with cells for 48 h. Following treatment, cells were washed once in PBS and fixed in 10% formalin for 20 min at room temperature. Methanol was used to permeabilize the cells for 10 min and PBS wash was carried out. Bovine serum albumin was used for 1 h blocking and primary antibodies for γH2AX (SCBT, #sc-517348) was incubated with the cells overnight. Fluorescent secondary antibodies were incubated with cells along with DAPI. Cells were washed trice with PBS and the slides were mounted prior to confocal imaging.

### Example 1: 5-FU Boronic Ester Prodrug Synthesis

The synthesis of boronic ester prodrug of 5-FU was conducted according to the synthetic route shown in Scheme 2. The N1-selective benzylation of 5-FU using compounds 2 and 3 in the presence of a base 1,8-diazabicyclo[5.4.0]undec-7-ene (DBU) generated phenylboronate ester 1a and control compound 1c. Then compound 1a was treated with NaIO₄ in the presence of NH₄OAc to yield compound 1b. Compound 1b is treated with various alcohols to provide prodrugs 1d-1j.

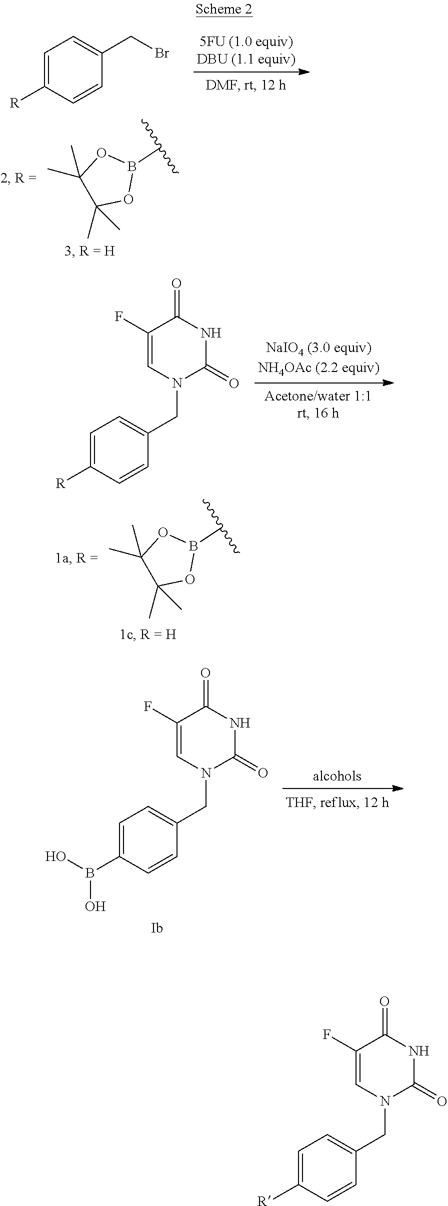

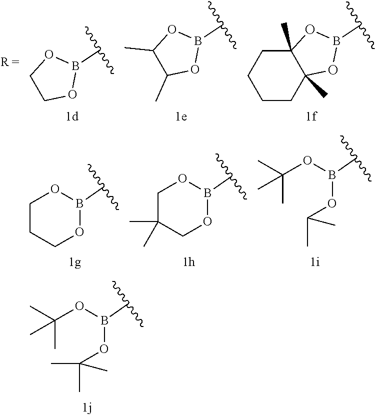

To a solution of 5-FU (130 mg, 1.0 mmol) and DBU (269 μL, 1.1 mmol) in dry DMF (4 ml) under N2 atmosphere at 4° C. was added compound 2 or 3 (1.0 mmol) dropwise as a solution in dry DMF (1 ml). The reaction mixture was allowed to stir at room temperature overnight, and then concentrated. The crude product was purified using flash chromatography (hexane/ethyl acetate=1:1 to 1:4) to get the corresponding products 1a and 1c.

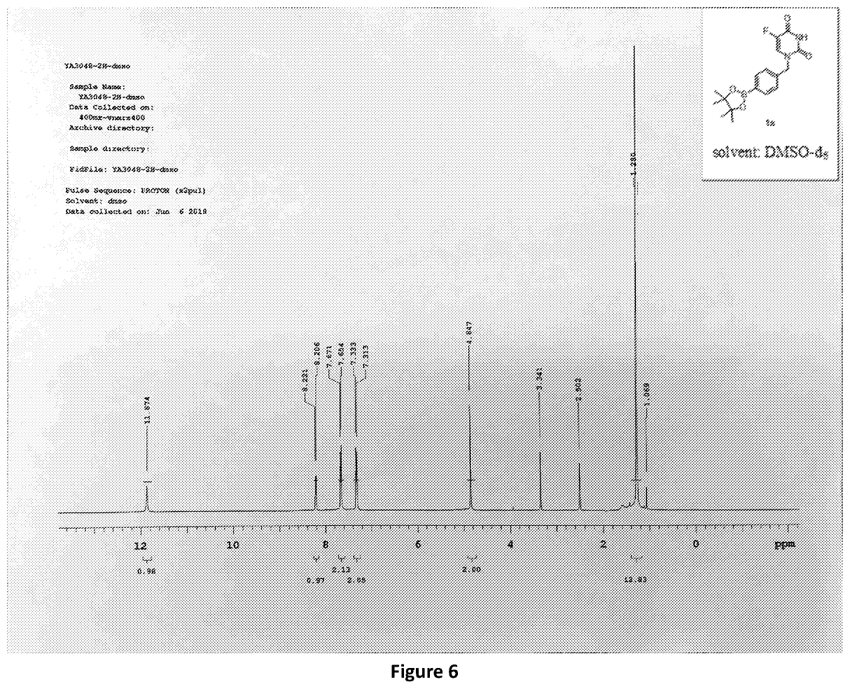

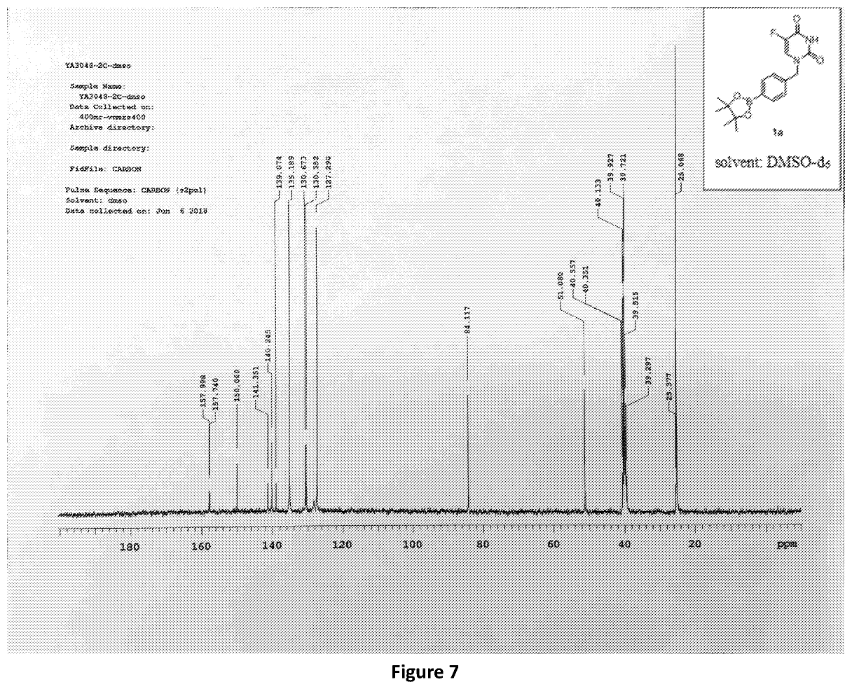

5-Fluoro-1-(4-(4,4,5,5-tetramethyl-1,3,2-dioxaborolan-2-yl)benzyl)pyrimidine-2,4(1H,3H)-dione (1a). White solid, 37% yield. See FIGS. 6 and 7 for NMR spectra. ¹H-NMR (400 MHz, DMSO-d6) δ 11.87 (s, 1H, CONHCO), 8.21 (d, J=6.0 Hz, 1H, FC═CH), 7.66 (d, J=8.0 Hz, 2H, 2×Ar-H), 7.32 (d, J=8.0 Hz, 2H, 2×Ar-H), 4.85 (s, 2H, Ar—CH2), 1.28 (s, 12H, 4×CH₃); ¹³C-NMR (100 MHz, DMSO-d6) δ 157.9 (d, J_{CF}=25.8 Hz), 150.1, 140.2 (d, J_{CF}=227.7 Hz), 140.2, 135.2, 130.5 (d, J_{CF}=32.1 Hz), 127.3, 84.1, 51.1, 25.1; HRMS (ESI): calcd. for C₁₇H₂₁BFN₂O₄ [M+H]⁺ 347.1578, found 34.1575.

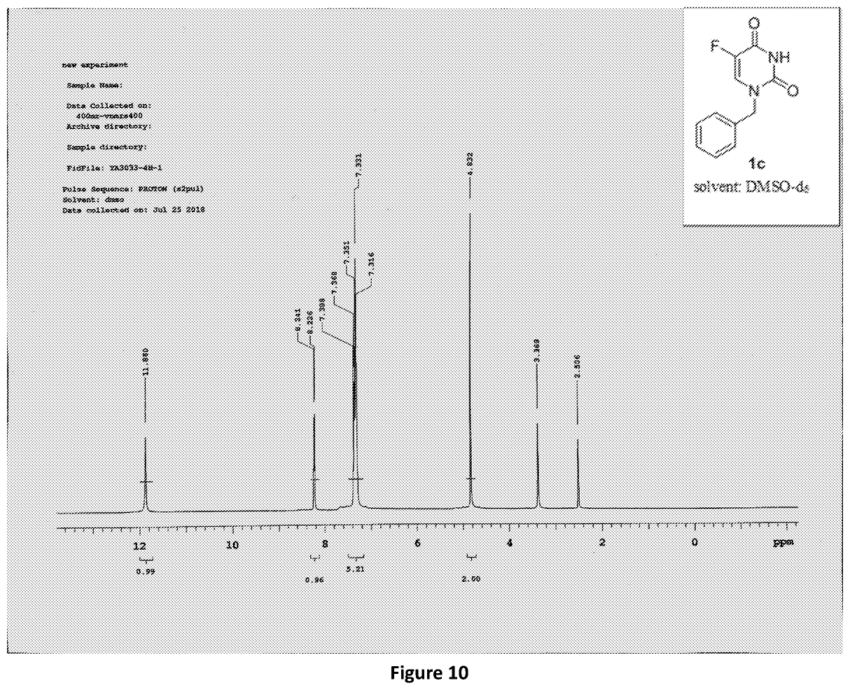

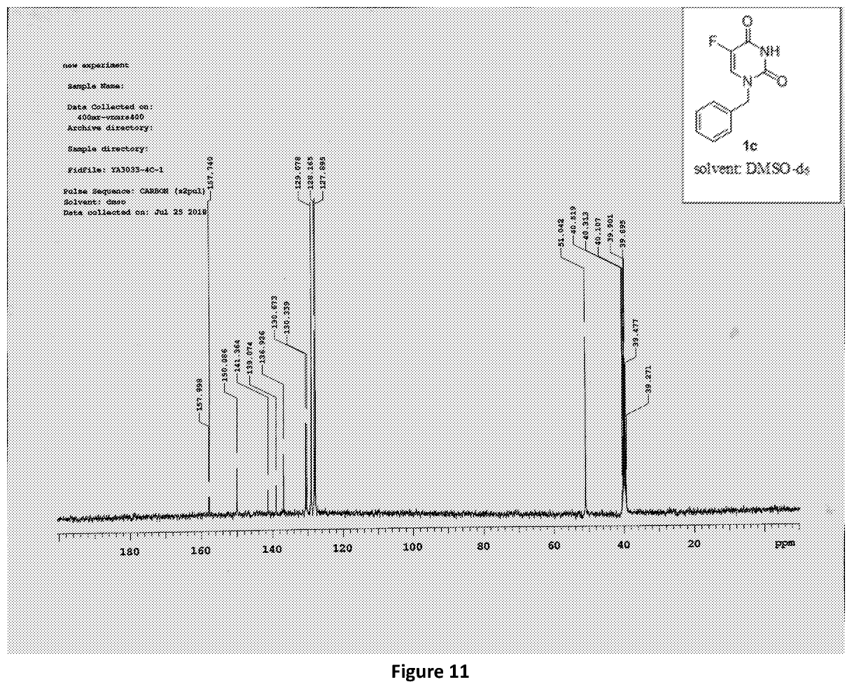

1-Benzyl-5-fluoropyrimidine-2,4(1H,3H)-dione (1c). White solid, 49% yield. See FIGS. 10 and 11 for NMR spectra. ¹H-NMR (400 MHz, DMSO-d6) δ 11.86 (s, 1H, CONHCO), 8.24 (d, J=6.0 Hz, 1H, NCH═CF), 7.39-7.32 (m, 5H, 5×Ar-H), 4.83 (s, 2H, Ar—CH₂); ¹³C-NMR (100 MHz, DMSO-d6) δ 157.8 (d, J_{CF}=25.8 Hz), 150.1, 140.2 (d, J_{CF}=229.0 Hz), 136.9, 130.5 (d, J_{CF}=33.4 Hz), 129.1, 127.9, 51.0; HRMS (ESI): calcd. for C₁₁H₁₀FN₂O₂ [M+H]⁺ 221.0726, found 221.0728.

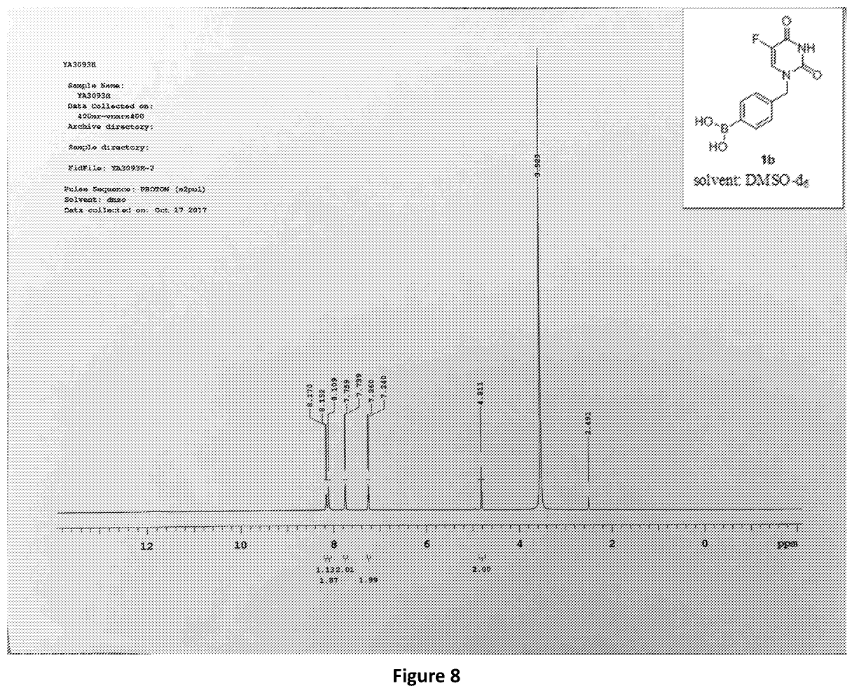

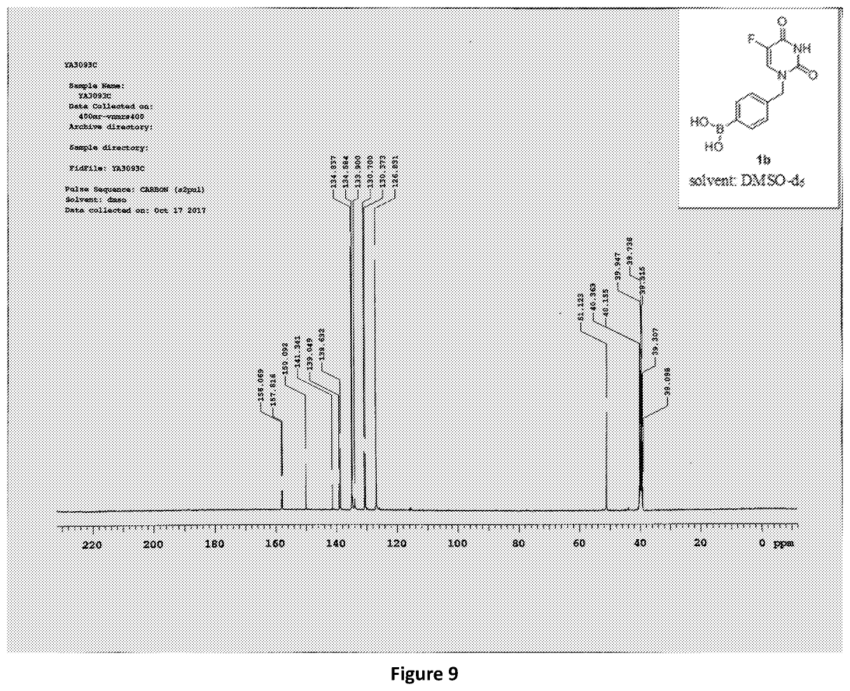

(4-((5-Fluoro-2,4-dioxo-3,4-dihydropyrimidin-I(2H)-yl)methyl)phenyl)boronic acid (1b). To a solution of the ester 1a (346 mg, 1.0 mmol) in acetone (5 mL) were added NaIO₄ (642 mg, 3.0 mmol), NH₄OAc (169 mg, 2.2 mmol), and water (5 mL), and the mixture was stirred for 16 h. Solvents were then removed under reduced pressure and the crude purified using flash chromatography (5% MeOH in DCM). White solid, 69% yield. See FIGS. 8 and 9 for NMR spectra. ¹H-NMR (400 MHz, DMSO-d6) δ 11.86 (br s, 1H, CONHCO), 8.16 (d, J=7.2 Hz, 1H, FC═CH), 8.11 (s, 2H, 2×OH), 7.75 (d, J=8.0 Hz, 2H, 2×Ar-H), 7.25 (d, J=8.0 Hz, 2H, 2×Ar-H), 4.81 (s, 2H, Ar—CH₂); ¹³C-NMR (100 MHz, DMSO-d6) δ 157.9 (d, J_{CF}=25.3 Hz), 150.1, 140.2 (d, J_{CF}=229.2 Hz), 138.6, 134.8, 134.6, 133.9, 130.5 (d, J_{CF}=32.7 Hz), 126.8, 51.1; HRMS (ESI): calcd. for C₁₁H₉BFN₂O₄ [M+H]⁺ 263.0639, found 263.0638.

### Example 2: 5-FU Release Studies

Release of 5-FU from the prodrugs was performed at 37° C. in 30% DMSO/Phosphate Buffered saline (PBS). The compounds were co-incubated at 100 μM together with hydrogen peroxide (H₂O₂) in triplicates. At each time point, aliquots were taken from the solution and diluted in acetonitrile for quantitation in the LC/MS. LC/MS Analysis: samples were analyzed using Xevo TQ-XS Triple Quadrupole Mass Spectrometry (Waters Corp, Milford, Mass.). The column used was a Cortecs C18+2.7 μm 2.1×75 mm column. A 3-min gradient elution with 0.5 mL/min flow rate was used for the samples. Running gradient: 95% mobile phase A over 1.2 min, 95% mobile phase B over 1.2 min, and 95% A 0.6 min, with elution at 1.64 min for the compounds. The ion transitions monitored for the compounds were 5-FU (129.0>40.1) 1a (345.2>86.0), and 1b (263.1>176.1).

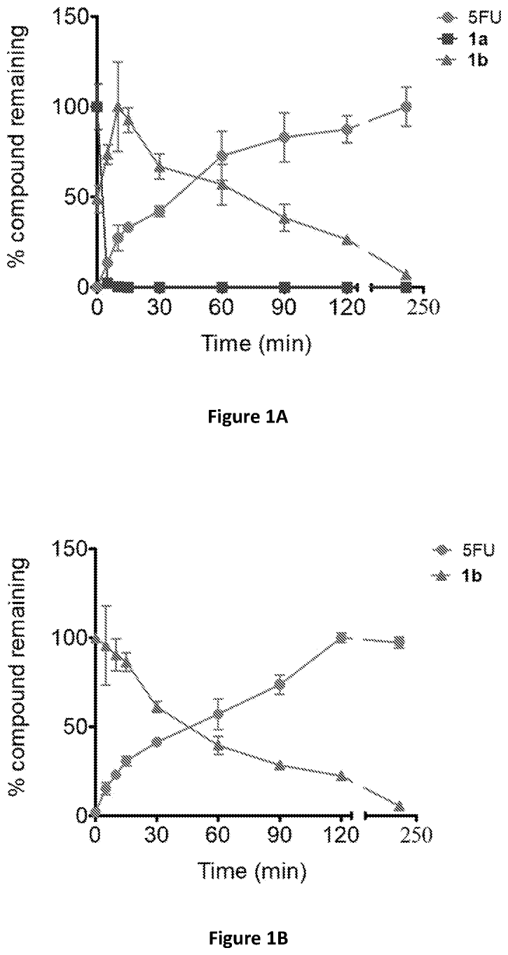

Compounds 1a-1b were incubated in a mixture of DMSO in PBS (30%) with the addition of H₂O₂ (5 equiv) at 37° C. The conversion of prodrugs 1a-1b and the production of 5-FU were followed using RP-UPLC-MS. Both prodrugs were activated efficiently by H₂O₂ to release 5-FU. Under the assay conditions, the boronic ester 1a hydrolyzes rapidly to generate boronic acid 1b (FIGS. 1A and 1B).

### Example 3: NCI-60 Cellular Antitumor Activity Profiling

The anticancer effects of compounds 1a-1b against 60 human cancer cell lines (the Developmental Therapeutics Program of NCI) were studied. The cell lines were derived from different cancer types including leukemia, non-small-cell lung cancer, colon cancer, CNS cancer, melanoma, ovarian cancer, renal cancer, prostate cancer, and breast cancer (Table 1; the assay protocol can be found at: https://dtp.cancer.gov/default.htm). Compound 1a caused over 50% growth inhibition for most tested cell lines. In particular, it exhibited most significant inhibition (81-96%) against the lung cancer NCI-H522 cells, melanoma MDA-MB-435 cells, ovarian cancer OVCAR-3 cells, and breast cancer MCF7 and MDA-MB-468 cells (Table 2). Boronic ester 1a demonstrated larger effects than the boronic acid analog 1b (less than 50% growth inhibition for most cell lines, Table 1). Without wishing to be bound by any particular theory, it is believed that the differences in the anticancer effects between compounds 1a and 1b may be due to the more favorable cell membrane permeability of the boronic ester 1a.

TABLE 1
NCI-60 Cellular Antitumor Activity Profiling

| | | Growth Inhibition (%)^{a} | |
|---|---|---|---|
| Tumor Type | Cell Line | 1a | 1b |
| Leukemia | CCRF-CEM | 62 | 7 |
| | HL-60(TB) | 68 | 11 |
| | K-562 | 70 | 18 |
| | MOLT-4 | 45 | 5 |
| | RPMI-8226 | 32 | 2 |
| | SR | 60 | 16 |
| Non-Small Cell | A549/ATCC | 24 | 4 |
| lung Cancer | EKVX | 28 | 16 |
| | HOP-62 | 36 | 10 |
| | HOP-92 | 67 | 35 |
| | NCI-H226 | 36 | 19 |
| | NCI-H23 | 32 | 6 |
| | NCI-H322M | 14 | 12 |
| | NCI-H460 | 66 | 1 |
| | NCI-H522 | 90 | 44 |
| Colon Cancer | COLO 205 | 64 | 8 |
| | HCC-2998 | 26 | 5 |
| | HCT-116 | 71 | 18 |
| | HCT-15 | 32 | 10 |
| | HT29 | 76 | 12 |
| | KM12 | 74 | 20 |
| | SW-620 | 66 | 17 |
| CNS Cancer | SF-268 | 31 | 14 |
| | SF-295 | 14 | 5 |
| | SF-539 | 39 | 13 |
| | SNB-19 | 29 | 11 |
| | SNB-75 | 62 | 15 |
| | U251 | 17 | 4 |
| Melanoma | LOX IMVI | 47 | 8 |
| | MALME-3M | 55 | 32 |
| | M14 | 55 | 17 |
| | MDA-MB-435 | 96 | 65 |
| | SK-MEL-2 | 59 | 7 |
| | SK-MEL-5 | 61 | 10 |
| | UACC-257 | 33 | 14 |
| | UACC-62 | 71 | 30 |
| Ovarian Cancer | IGROV1 | 56 | 15 |
| | OVCAR-3 | 95 | 30 |
| | OVCAR-4 | 30 | 2 |
| | OVCAR-5 | 30 | 19 |
| | OVCAR-8 | 25 | 6 |
| | NCI/ADR-RES | 6 | 0 |
| | SK-OV-3 | 31 | 13 |
| Renal Cancer | 786-0 | 19 | 6 |
| | A498 | 74 | 31 |
| | ACHN | 8 | 0 |
| | CAKI-1 | 25 | 12 |
| | RXF 393 | 40 | 17 |
| | SN12C | 19 | 7 |
| | TK-10 | 21 | 20 |
| | UO-31 | 27 | 27 |
| Prostate Cancer | PC-3 | 45 | 14 |
| | DU-145 | 16 | −6 |
| Breast Cancer | MCF7 | 81 | 33 |
| | MDA-MB- | 14 | 13 |
| | 231/ATCC | | |
| | HS 578T | 63 | 26 |
| | BT-549 | 60 | 30 |
| | T-47D | 51 | 5 |
| | MDA-MB-468 | 87 | 22 |

^{a}Each cell line was treated with either compound 1a or compound 1b at the concentration of 50 μM for a period of 48 h. Then the growth inhibition (%) values were determined.

TABLE 2
Anticancer Effects of Compounds 1a and 1b Against Breast Cancer

| | | Growth | |
|---|---|---|---|
| | | Inhibition (%)^{a} | |
| Tumor type | Cell Line | 1a | 1b |
| Lung Cancer | NCI-H522 | 90 | 44 |
| Melanoma | MDA-MB-435 | 96 | 65 |
| Ovarian Cancer | OVCAR-3 | 95 | 30 |
| Breast Cancer | MCF7 | 81 | 33 |
| | MDA-MB-468 | 87 | 22 |

^{a}Each cell line was treated with either compound 1a or compound 1b at the concentration of 50 μM for a period of 48 h. Then the growth inhibition (%) values were determined.

### Example 4: Cellular Assay for Determining Anticancer Effects of ROS Activated 5-FU Prodrugs Against Breast Cancer MCF7 Cell Line

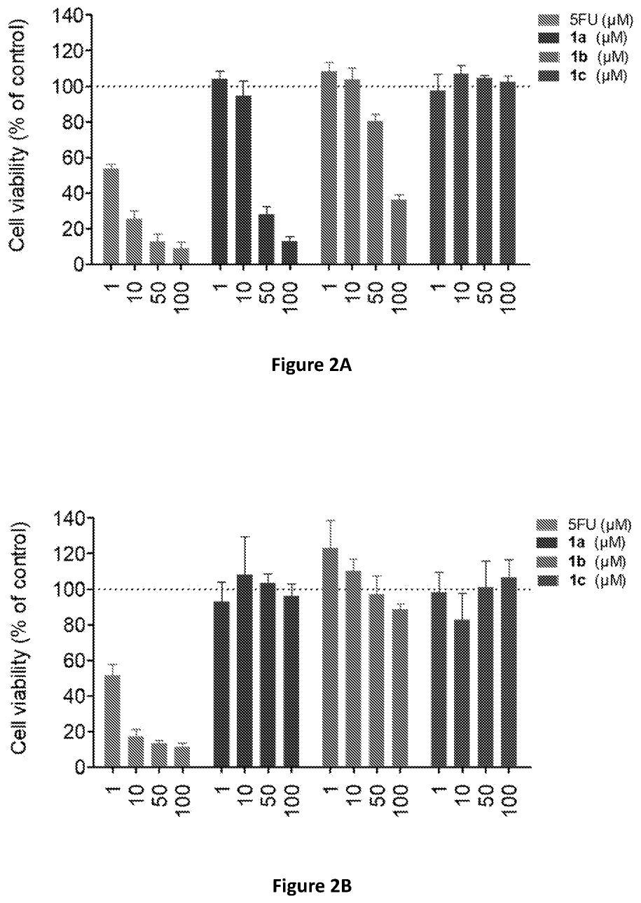

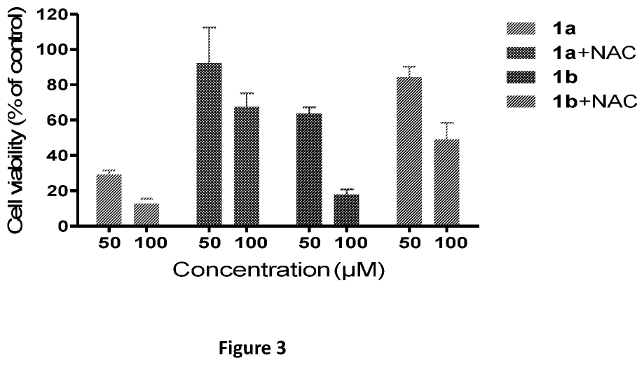

The anticancer effects of new compounds 1a and 1b were tested using the breast cancer MCF7 cells and normal MEC1 cells (FIGS. 2A and 2B). Compound 1a exerted comparable antiproliferative activity as that of the parent drug 5-FU. Compound 1b demonstrated similar but weaker anticancer effects for the MCF7 cells. Compared to 5-FU, both prodrugs 1a and 1b demonstrated no toxicity toward the normal MEC1 cells (cell viability >95%) at the concentration of 100 μM. These differential killing results highlight the specificity of new prodrugs 1a-1b for cancer cells over normal cells. Moreover, the control compound 1c, without the ROS-reactive group in its structure, indicated no obvious antiproliferative effects against the MCF7 cells, indicating that new prodrugs 1a and 1b achieved their anticancer effects through the reactivity of the boronic acid or ester groups towards the ROS. To further verify the role of ROS in the drug release process, the MTT assays was performed, using MCF7 cells pretreated with the ROS scavenger N-Acetyl-Cysteine (NAC). Upon pretreatment of NAC, the anticancer effects of prodrugs 1a-1b decreased significantly (FIG. 3), confirming that the new prodrugs exerted their cytotoxicity depending on ROS activation.

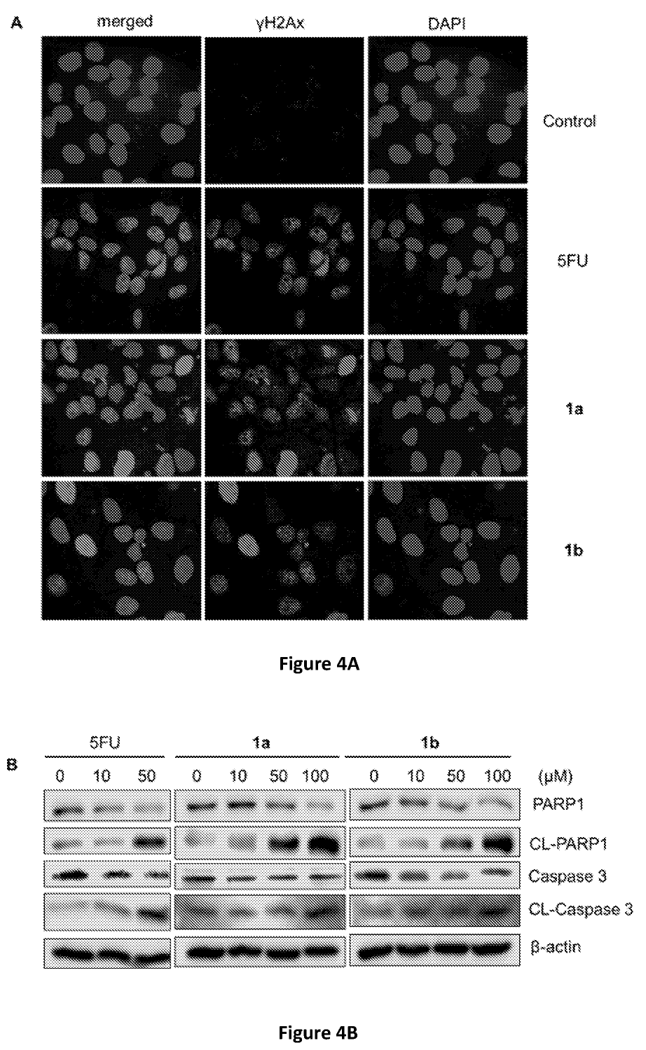

To understand the mechanism of action of prodrugs 1a-1b, the impact of the treatment of compounds 1a-1b on the γH2AX formation in MCF7 cells were determined using immunofluorescence assay (FIG. 4A). Treatment of MCF7 cells with compounds 1a-1b dramatically increased the γH2AX formation, which was similar to the effect of parent 5-FU. The effects of prodrugs 1a-1b on the PARP1 and caspase 3 cleavage in MCF7 cells were also determined using Western blots assays (FIG. 4B). Compared to vehicle-treated MCF7 cells, treatment of prodrugs 1a-1b, similar to 5-FU, significantly reduced the expression levels of PARP1 and caspase 3 in MCF7 cells. Treatment of 5-FU and compounds 1a-1b increased the levels of cleavage of PARP1 and caspase 3 in MCF7 cells, as compared with that in the vehicle-treated controls. Therefore, prodrugs 1a-1b, similar to 5-FU, can induce MCF7 cell apoptosis, which might contribute to their pharmacological actions.

### Example 5: Safety Profile Evaluation

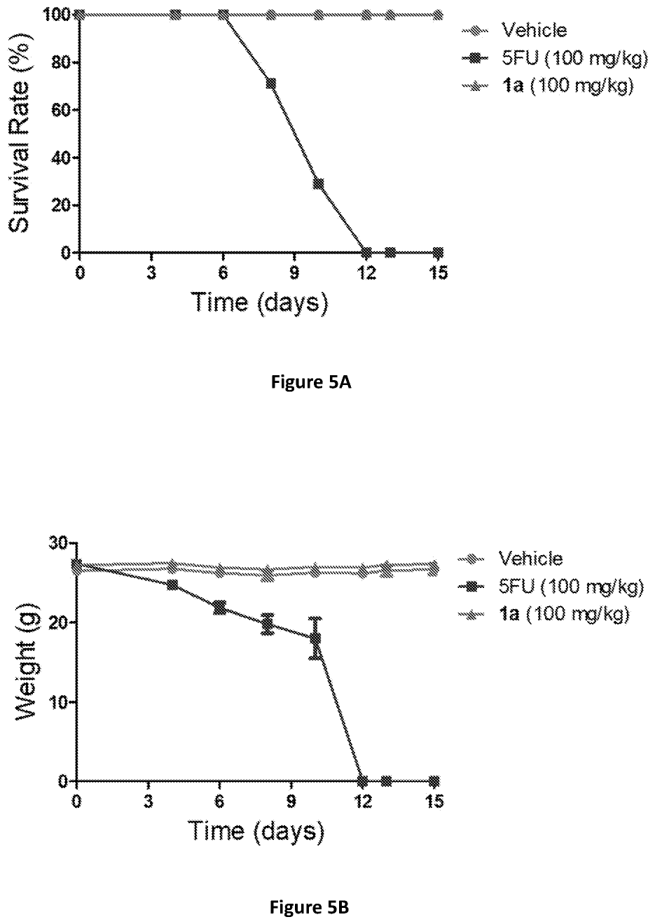

To evaluate the safety profile of the novel prodrugs, the acute toxicity of compound 1a in wild-type C57BL/6 male mice was tested (FIGS. 5A and 5B). Twenty-one mice were randomly distributed into three groups (seven mice in each group): vehicle (5% DMSO and 15% polyethylene glycol in saline), 100 mg/kg of 5-FU and 100 mg/kg of prodrug 1a. Mice were treated every other day by intraperitoneal (i.p.) injection for 15 days, the survival and body weights of the mice were recorded after treatment.

While treatment with a high dose (100 mg/kg) of 5-FU killed all mice on the twelfth day (FIG. 5A), the same dose of compound 1a did not cause animal death or abnormality in eating, drinking, or activity throughout the period. While body weights of the mice in the 5-FU treated group significantly decreased compared to those of the vehicle group, no obvious changes were observed in prodrug 1a treated group (FIG. 5B). These results indicate that, compared with the parent 5-FU, prodrug 1a had more favorable drug safety, exhibiting reduced toxicity in normal tissues.

### Example 6: Tumor Selective 6MP-Based Prodrugs Using a Reactive Oxygen Species-Activated Approach

Synthesis of prodrugs YA6075 began with 6MP (Scheme 3). Protection of the SH group of 6MP using bromodiphenylmethane in the presence of K₂CO₃ as a base, generated compound YA6065, which was further reacted with 2-(4-(bromomethyl)phenyl)-4,4,5,5-tetramethyl-1,3,2-dioxaborolane to provide compound YA6066. Deprotection of the diphenylmethyl group using TFA yields prodrug YA6075.

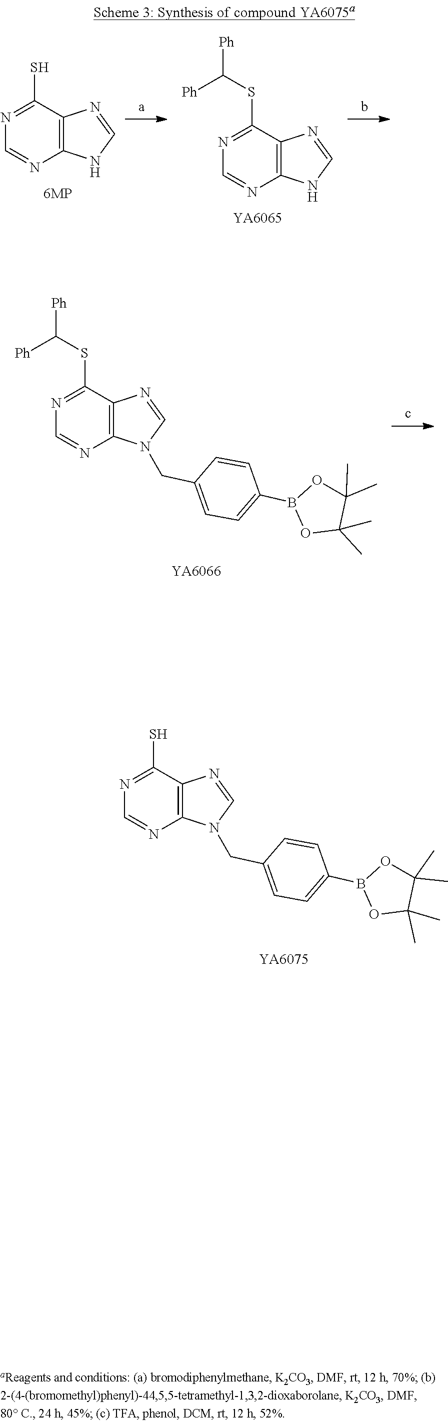

Synthesis of 6-(benzhydrylthio)-9H-purine (YA6065): to a solution of 6MP (9.9 mmol) in DMF was added K₂CO₃ (9.9 mmol). The solution was stirred at room temperature for 15 min. To the mixture was added bromodiphenylmethane (9.9 mmol), and the mixture was stirred at room temperature for 12 h. The mixture was diluted with water (200 mL), and then acidified with acetic acid. The mixture was stirred vigorously for 30 min, filtered, and then dried to give an amorphous solid. Recrystallization from MeOH gave 2.2 g (70%) of compound YA6065 as white powder. ¹H-NMR (400 MHz, DMSO-d6) δ 13.56 (br s, 1H, NH), 8.65 (s, 1H), 8.47 (s, 1H), 7.51 (d, J=6.8 Hz, 4H), 7.32 (t, J=8.0, 6.8 Hz, 4H), 7.23 (t, J=7.2 Hz, 2H), 6.73 (s, 1H); ¹³C-NMR (100 MHz, DMSO-d6) δ 151.8, 144.0, 141.7, 129.0, 128.6, 127.7, 50.6.

Synthesis and Characterization of Compound YA6066 (6-(Benzhydrylthio)-9-(4-(4,4,5,5-tetramethyl-1,3,2-dioxaborolan-2-yl)benzyl)-9H-purine (YA6066)): to a solution of compound YA6065 (1 mmol) in DMF was added K₂CO₃ (1 mmol) and 2-(4-(bromomethyl)phenyl)-4,4,5,5-tetramethyl-1,3,2-dioxaborolane (1 mmol) at room temperature. The reaction mixture was allowed to stir at 80° C. for 24 h and then concentrated. The crude product was purified using flash chromatography to get the corresponding product YA6066. ¹H-NMR (400 MHz, CDCl₃) δ 8.76 (s, 1H), 7.95 (s, 1H), 7.85 (d, J=7.6 Hz, 2H), 7.60 (d, J=6.8 Hz, 4H), 7.36 (t, J=8.0, 6.8 Hz, 4H), 7.29 (t, J=6.8 Hz, 4H), 6.91 (s, 1H), 5.42 (s, 2H), 1.40 (s, 12H); ¹³C-NMR (100 MHz, CDCl₃) δ 159.8, 152.1, 148.8, 142.5, 141.2, 137.9, 135.5, 130.8, 128.6, 128.5, 127.2, 127.0, 84.0, 50.7, 47.3, 24.9.

Synthesis and Characterization of Prodrug YA6075 (9-(4-(4,4,5,5-Tetramethyl-1,3,2-dioxaborolan-2-yl)benzyl)-9H-purine-6-thiol (YA6075)): deprotection of diphenylmethyl group from YA6066 was performed with 50% TFA/CH₂Cl₂ and phenol (3.0 equiv). The reaction mixture was allowed to stir at room temperature for 12 h and then concentrated. The crude product was purified using flash chromatography to get the prodrug YA6075. ¹H-NMR (400 MHz, DMSO-d6) δ 13.77 (s, 1H), 8.41 (s, 1H), 8.19 (s, 1H), 7.64 (d, J=8.0 Hz, 2H), 7.28 (d, J=8.0 Hz, 2H), 5.44 (s, 2H), 1.27 (s, 12H); ¹³C-NMR (100 MHz, DMSO-d6) δ 176.4, 145.6, 144.5, 143.6, 140.1, 135.4, 135.3, 127.3, 84.1, 47.1, 25.1; HRMS (ESI): m/z [M+H]⁺ calcd for C₁₈H₂₂BN₄O₂S, 369.1557, found, 369.1562.

Prodrug YA6131 was synthesized by reacting 6MP with 2-(4-(bromomethyl)phenyl)-4,4,5,5-tetramethyl-1,3,2-dioxaborolane using K₂CO₃ as a base (Scheme 4).

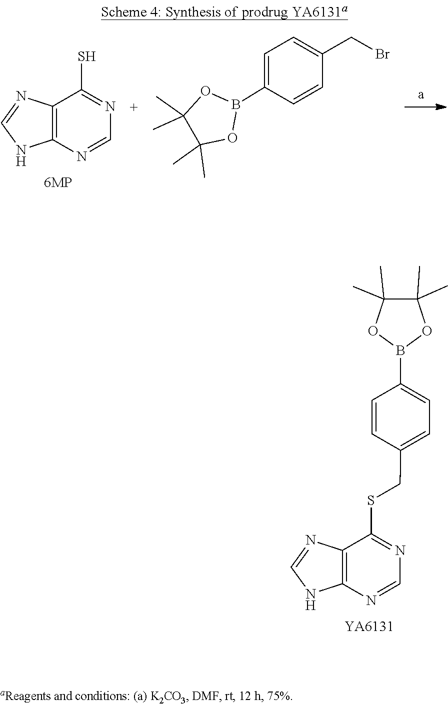

Synthesis and Characterization of Prodrug YA6131: to a solution of 6MP (1.0 mmol) and K₂CO₃ (1.1 mmol) in DMF (4 ml) under N2 atmosphere at 4° C. was added 2-(4-(bromomethyl)phenyl)-4,4,5,5-tetramethyl-1,3,2-dioxaborolane (1.0 mmol) dropwise as a solution in dry DMF (1 ml). The reaction mixture was allowed to stir at room temperature overnight, and then concentrated. The crude product was purified using flash chromatography to get the corresponding product YA6131. ¹H-NMR (400 MHz, CDCl₃) δ 8.78 (s, 1H), 8.26 (s, 1H), 7.74 (d, J=4.2 Hz, 2H), 7.47 (d, J=6.0 Hz, 2H), 4.67 (s, 2H), 1.32 (s, 12H); ¹³C-NMR (100 MHz, CDCl₃) δ 151.4, 141.4, 140.4, 135.0, 128.6, 83.8, 33.0, 24.8; HRMS (ESI): calcd. for C₁₈H₂₂BN₄O₂S [M+H]⁺ 369.1557, found 369.1555.

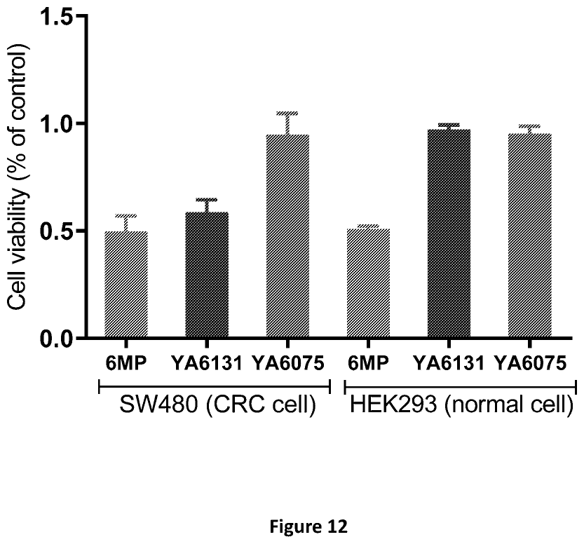

Anticancer effects and selectivity of 6MP (10 μM), prodrugs YA6131 (10 μM) and YA6075 (10 μM): the anticancer activities of prodrugs YA6131 and YA6075 were evaluated in comparison to the parent drug 6MP using the human colorectal cancer cells SW480 and normal HEK293 cells (FIG. 12). Prodrug YA6131 exerted similarly potent anti-proliferative activity as compared to 6MP, while prodrug YA6075 has a reduced effect. Without wishing to be bound by any particular theory, it was reasoned that the difference of the anticancer effects of the two prodrugs were due to their different release kinetics of the two compounds in the particular cells used.

A number of patent and non-patent publications are cited herein in order to describe the state of the art to which this invention pertains. The entire disclosure of each of these publications is incorporated by reference herein.

While certain embodiments of the present invention have been described and/or exemplified above, various other embodiments will be apparent to those skilled in the art from the foregoing disclosure. The present invention is, therefore, not limited to the particular embodiments described and/or exemplified, but is capable of considerable variation and modification without departure from the scope and spirit of the appended claims.

### REFERENCES

Each reference cited below is hereby incorporated in its entirety as if fully set forth herein.
(1) Heidelberger, C.; Chaudhuri, N. K.; Danneberg, P.; Mooren, D.; Griesbach, L.; Duschinsky, R.; Schnitzer, R. J.; Pleven, E.; Schemer, J. Nature 1957, 179, 663.
(2) Longley, D. B.; Harkin, D. P.; Johnston, P. G. Nat. Rev. Cancer 2003, 3, 330.
(3) Macdonald, J. S. Oncology (Williston Park) 1999, 13, 33.
(4) Heggie, G. D.; Sommadossi, J. P.; Cross, D. S.; Huster, W. J.; Diasio, R. B. Cancer Res. 1987, 47, 2203.
(5) Shelton, J.; Lu, X.; Hollenbaugh, J. A.; Cho, J. H.; Amblard, F.; Schinazi, R. F. Chem. Rev. 2016, 116, 14379.
(6) Ikeda, K.; Yoshisue, K.; Matsushima, E.; Nagayama, S.; Kobayashi, K.; Tyson, C. A.; Chiba, K.; Kawaguchi, Y. Clin. Cancer Res. 2000, 6, 4409.
(7) El Sayed, Y. M.; Sadee, W. Cancer Res. 1983, 43, 4039.
(8) Schoffski, P. Anticancer Drugs 2004, 15, 85.
(9) Kono, A.; Hara, Y.; Sugata, S.; Karube, Y.; Matsushima, Y.; Ishitsuka, H. Chem. Pharm. Bull. 1983, 31, 175.
(10) Nishioka, M.; Miyamoto, H.; Kurita, N.; Higashijima, J.; Yoshikawa, K.; Miyatani, T.; Shimada, M. Hepatogastroenterology 2007, 54, 1089.
(11) Sakamoto, J.; Oba, K.; Matsui, T.; Kobayashi, M. Dis. Colon Rectum 2006, 49, S82.
(12) Shimma, N.; Umeda, I.; Arasaki, M.; Murasaki, C.; Masubuchi, K.; Kohchi, Y.; Miwa, M.; Ura, M.; Sawada, N.; Tahara, H.; Kuruma, I.; Horii, I.; Ishitsuka, H. Bioorg. Med. Chem. 2000, 8, 1697.
(13) Hileman, E. O.; Liu, J.; Albitar, M.; Keating, M. J.; Huang, P. Cancer Chemother. Pharmacol. 2004, 53, 209.
(14) Szatrowski, T. P.; Nathan, C. F. Cancer Res. 1991, 51, 794.
(15) Toyokuni, S.; Okamoto, K.; Yodoi, J.; Hiai, H.: Persistent oxidative stress in cancer. FEBS lett. 1995, 358, 1-3.
(16) Ushio-Fukai, M.; Alexander, R. W. Mol. Cell. Biochem. 2004, 264, 85.
(17) Ishikawa, K.; Takenaga, K.; Akimoto, M.; Koshikawa, N.; Yamaguchi, A.; Imanishi, H.; Nakada, K.; Honma, Y.; Hayashi, J. Science 2008, 320, 661.
(18) Dharmaraja, A. T. J. Med. Chem. 2017, 60, 3221.
(19) Arnold, R. S.; Shi, J.; Murad, E.; Whalen, A. M.; Sun, C. Q.; Polavarapu, R.; Parthasarathy, S.; Petros, J. A.; Lambeth, J. D. Proc. Natl. Acad. Sci. U.S.A. 2001, 98, 5550.
(20) Pelicano, H.; Feng, L.; Zhou, Y.; Carew, J. S.; Hileman, E. O.; Plunkett, W.; Keating, M. J.; Huang, P. J. Biol. Chem. 2003, 278, 37832.
(21) Trachootham, D.; Alexandre, J.; Huang, P. Nat. Rev. Drug Discov. 2009, 8, 579.
(22) Fang, J.; Seki, T.; Maeda, H. Adv. Drug Deliv. Rev. 2009, 61, 290.
(23) Brown, J. M.; Wilson, W. R. Nat. Rev. Cancer 2004, 4, 437.
(24) Peng, X.; Gandhi, V. Ther. Deliv. 2012, 3, 823.
(25) Liao, Y.; Xu, L.; Ou, S.; Edwards, H.; Luedtke, D.; Ge, Y.; Qin, Z. ACS Med. Chem. Lett. 2018, 9, 635.
(26) Peiro Cadahia, J.; Bondebjerg, J.; Hansen, C. A.; Previtali, V.; Hansen, A. E.; Andresen, T. L.; Clausen, M. H. J. Med. Chem. 2018, 61, 3503.
(27) Kim, E. J.; Bhuniya, S.; Lee, H.; Kim, H. M.; Cheong, C.; Maiti, S.; Hong, K. S.; Kim, J. S. J. Am. Chem. Soc. 2014, 136, 13888.
(28) Chen, W.; Balakrishnan, K.; Kuang, Y.; Han, Y.; Fu, M.; Gandhi, V.; Peng, X. J. Med. Chem. 2014, 57, 4498.
(29) Kuang, Y.; Balakrishnan, K.; Gandhi, V.; Peng, X. J. Am. Chem. Soc. 2011, 133, 19278.
(30) Dharmaraja, A. T.; Ravikumar, G.; Chakrapani, H. Org. Lett. 2014, 16, 2610.
(31) Ravikumar, G.; Bagheri, M.; Saini, D. K.; Chakrapani, H. Chem. Commun. 2017, 53, 13352.
(32) Marzenell, P.; Hagen, H.; Sellner, L.; Zenz, T.; Grinyte, R.; Pavlov, V.; Daum, S.; Mokhir, A. J. Med. Chem. 2013, 56, 6935.
(33) Hagen, H.; Marzenell, P.; Jentzsch, E.; Wenz, F.; Veldwijk, M. R.; Mokhir, A. J. Med. Chem. 2012, 55, 924.
(34) Daum, S.; Chekhun, V. F.; Todor, I. N.; Lukianova, N. Y.; Shvets, Y. V.; Sellner, L.; Putzker, K.; Lewis, J.; Zenz, T.; de Graaf, I. A.; Groothuis, G. M.; Casini, A.; Zozulia, O.; Hampel, F.; Mokhir, A. J. Med. Chem. 2015, 58, 2015.
(35) Major Jourden, J. L.; Cohen, S. M. Angew. Chem., Int. Ed. 2010, 49, 6795.
(36) Li, P.; Cavallero, S.; Gu, Y.; Chen, T. H.; Hughes, J.; Hassan, A. B.; Brüning, J. C.; Pashmforoush, M.; Sucov, H. M. Development 2011, 138, 1795.
(37) Carroll, F. I.; Philip, A. J. Org. Chem. 1968, 33, 3776-3779.
(38) Ko, E.; Kamkaew, A.; Burgess, K. Small molecule ligands for active targeting of TrkC-expressing tumor cells. ACS Med. Chem. Lett. 2012, 3, 1008-1012.

1. A compound of Formula I, or a pharmaceutically acceptable salt, solvate, hydrate, cocrystal, or prodrug thereof: 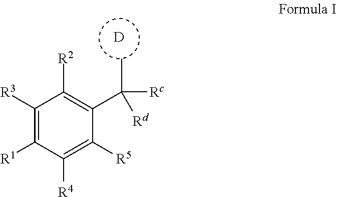
wherein in Formula I:
D is a drug or prodrug moiety comprising at least one heterocycle;
R¹, R², R³, R⁴, and R⁵ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted alkynyl, substituted or unsubstituted heteroalkynyl, substituted or unsubstituted cycloalkynyl, substituted or unsubstituted heterocycloalkynyl, substituted or unsubstituted aryl, substituted or unsubstituted heteroaryl, hydroxy, halo, cyano, trifluoromethyl, trifluoromethoxy, nitro, trimethylsilanyl, —OR^{a}, —B(OR^{a})OR^{b}, —SR^{a}, —OC(O)—R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{b}, —N(R^{a})C(O)R^{b}, —N(R^{a})C(O)N(R^{a})R^{b}, —N(R^{a})C(NR^{a})N(R^{a})R^{b}, —N(R^{a})S(O)R, —S(O)ₜR^{a}, —S(O)ₜOR^{a}, —S(O)ₜN(R^{a})R^{b}, and —PO(OR^{a})(OR^{b}), wherein at least one of R¹, R², R³, R⁴, or R⁵ is —OR^{a} or —B(OR^{a})OR^{b};
R^{a}, R^{b}, R^{c}, and R^{d} are independently selected at each occurrence from hydrogen, substituted or unsubstituted alkyl, substituted or unsubstituted fluoroalkyl, substituted or unsubstituted carbocyclyl, substituted or unsubstituted carbocyclylalkyl, substituted or unsubstituted aryl, substituted or unsubstituted aralkyl, substituted or unsubstituted heterocycloalkyl, substituted or unsubstituted heterocycloalkylalkyl, substituted or unsubstituted heteroaryl, or substituted or unsubstituted heteroarylalkyl;
t is 1 or 2;
wherein any of R¹, R², R³, R⁴, R⁵, R^{a}, R^{b}, R^{c}, and R^{d} can optionally be linked together.

2. The compound of claim 1, wherein R¹ is —OR^{a} or —B(OR^{a})OR^{b}.

3. The compound of claim 1, wherein R² is —OR^{a} or —B(OR^{a})OR^{b}, or wherein R⁵ is —OR^{a} or —B(OR^{a})OR^{b}.

4. The compound of claim 1, wherein the compound has Formula 11, Formula 12, Formula 13, Formula 14, or Formula 15: 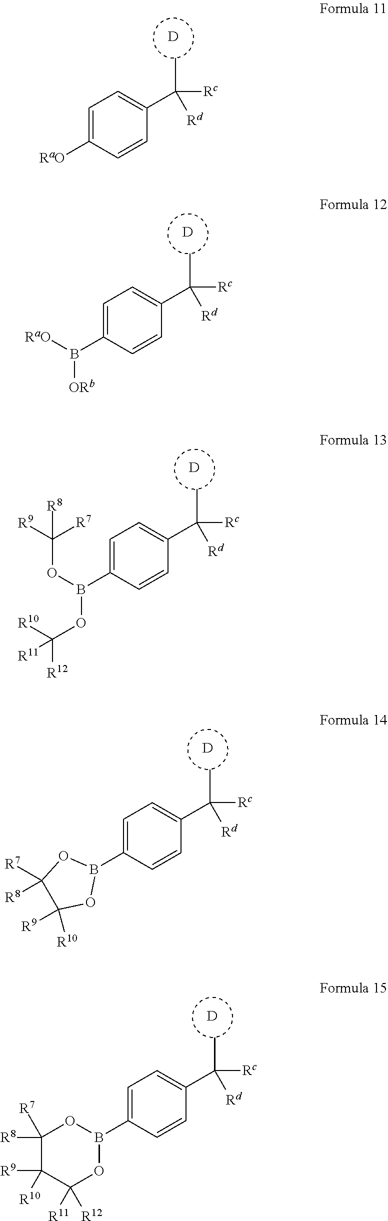
wherein in Formulas 11-15:
R⁷, R⁸, R⁹, R¹⁰, R¹¹, and R¹² are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted alkynyl, substituted or unsubstituted heteroalkynyl, substituted or unsubstituted cycloalkynyl, substituted or unsubstituted heterocycloalkynyl, substituted or unsubstituted aryl, substituted or unsubstituted heteroaryl, hydroxy, halo, cyano, trifluoromethyl, trifluoromethoxy, nitro, trimethylsilanyl, —OR^{a}, —SR^{a}, —OC(O)—R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{b}, —N(R^{a})C(O)R^{b}, —N(R^{a})C(O)N(R^{a})R^{b}, —N(R^{a})C(NR^{a})N(R^{a})R^{b}, —N(R^{a})S(O)ₜR^{b}, —S(O)ₜR^{a}, —S(O)ₜOR^{a}, —S(O)ₜN(R^{a})R^{b}, and —PO(OR^{a})(OR^{b});
R^{a}, R^{b}, R^{c}, and R^{d} are independently selected from hydrogen, substituted or unsubstituted alkyl, substituted or unsubstituted fluoroalkyl, substituted or unsubstituted carbocyclyl, substituted or unsubstituted carbocyclylalkyl, substituted or unsubstituted aryl, substituted or unsubstituted aralkyl, substituted or unsubstituted heterocycloalkyl, substituted or unsubstituted heterocycloalkylalkyl, substituted or unsubstituted heteroaryl, or substituted or unsubstituted heteroarylalkyl;
t is 1 or 2; and
wherein any of R⁷, R⁸, R⁹, R¹⁰, R¹¹, R¹², R^{a}, and R^{b} can optionally be linked together.

5. The compound of claim 4, wherein:
R⁷, R⁸, R⁹, R¹⁰, R¹¹, R¹², R^{a}, and R^{b} are independently selected from hydrogen, and substituted or unsubstituted alkyl.

6. The compound of claim 1, wherein at least one of R^{c} and R^{d} is hydrogen.

7. The compound of claim 1, wherein both R^{c} and R^{d} are hydrogen.

8. The compound of claim 1, wherein the compound has a formula selected from:

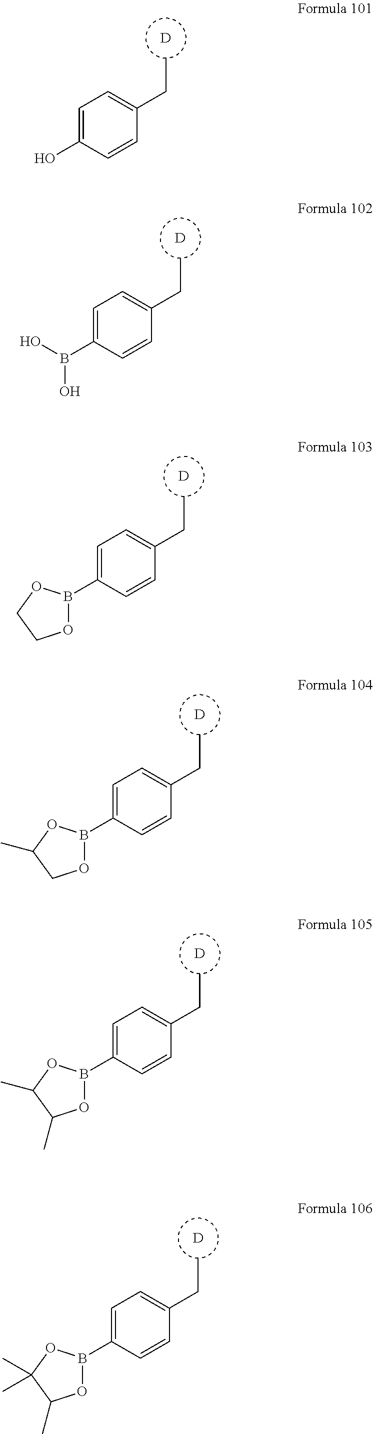

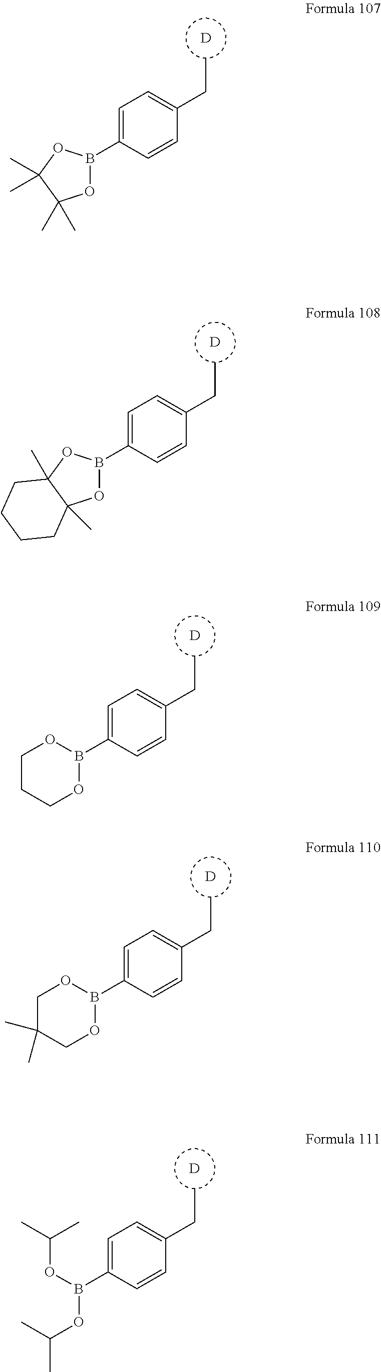

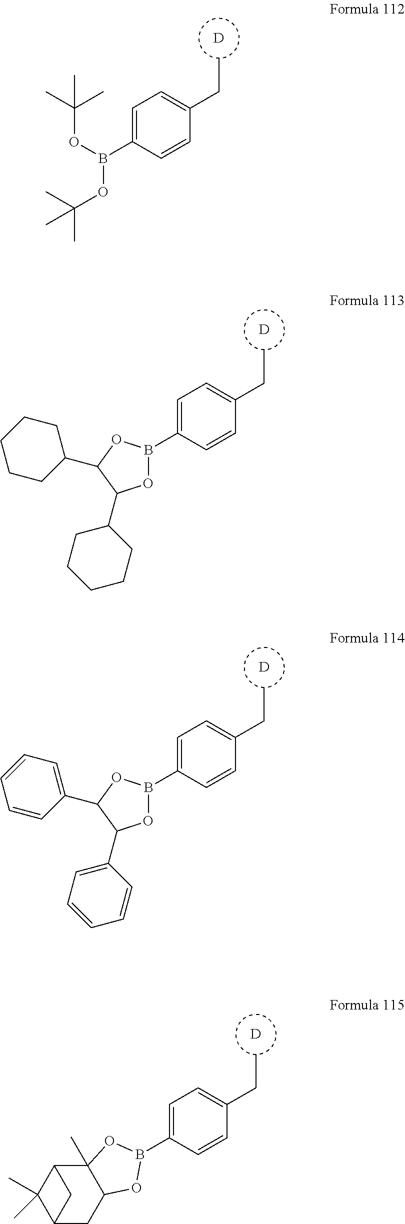

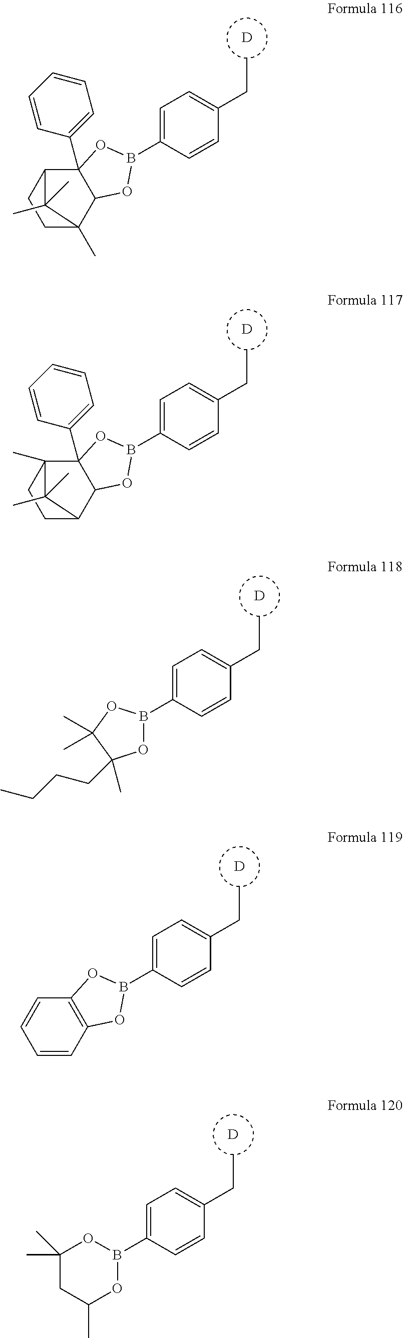

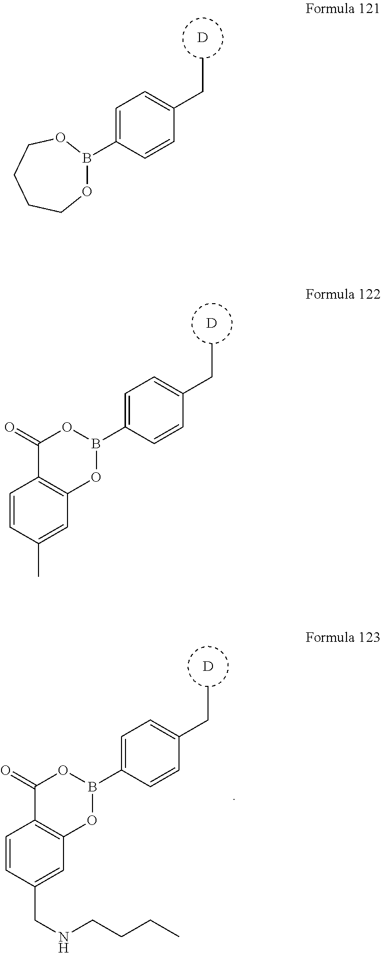

9. The compound of claim 1, wherein D comprises a pyrimidine moiety.

10. The compound of claim 1, wherein D is

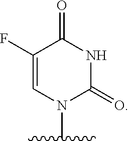 [SMILES: *n1cc(F)c(=O)[nH]c1=O]

11. The compound of claim 1, wherein D is 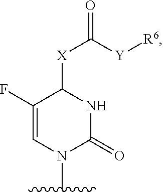
X and Y are independently selected from —O— and —NR^{a}—, and R⁶ is selected from hydrogen, substituted or unsubstituted alkyl, and substituted or unsubstituted heteroalkyl.

12. The compound of claim 1, wherein D comprises a purine moiety.

13. The compound of claim 1, wherein D is

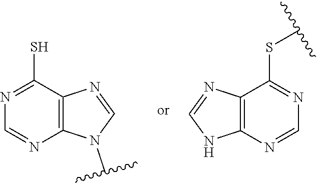 [SMILES: *n1cnc2c(S)ncnc21 ; *Sc1ncnc2[nH]cnc12]

14. The compound of claim 1, wherein D comprises a moiety selected from:

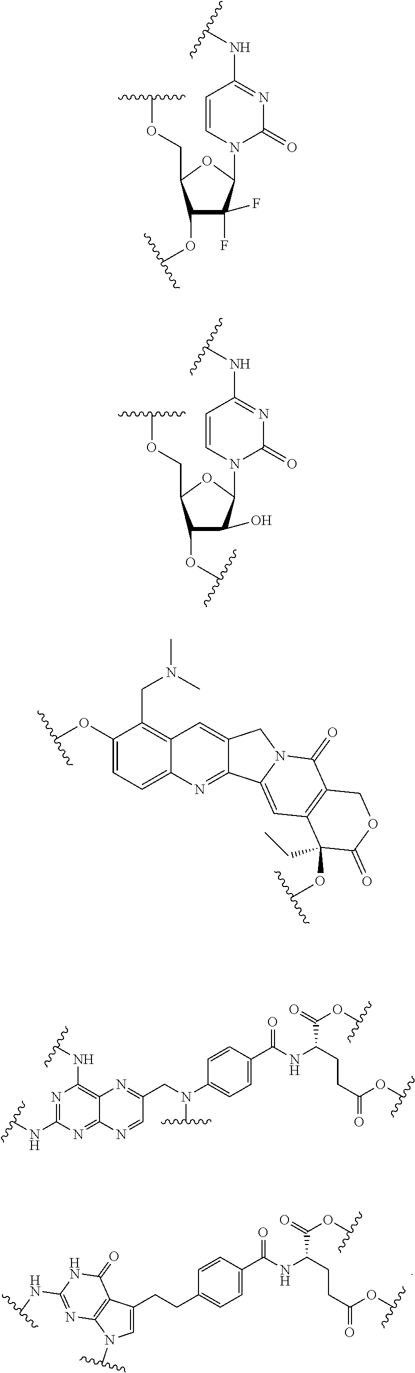

15. The compound of claim 1, wherein D is selected from:

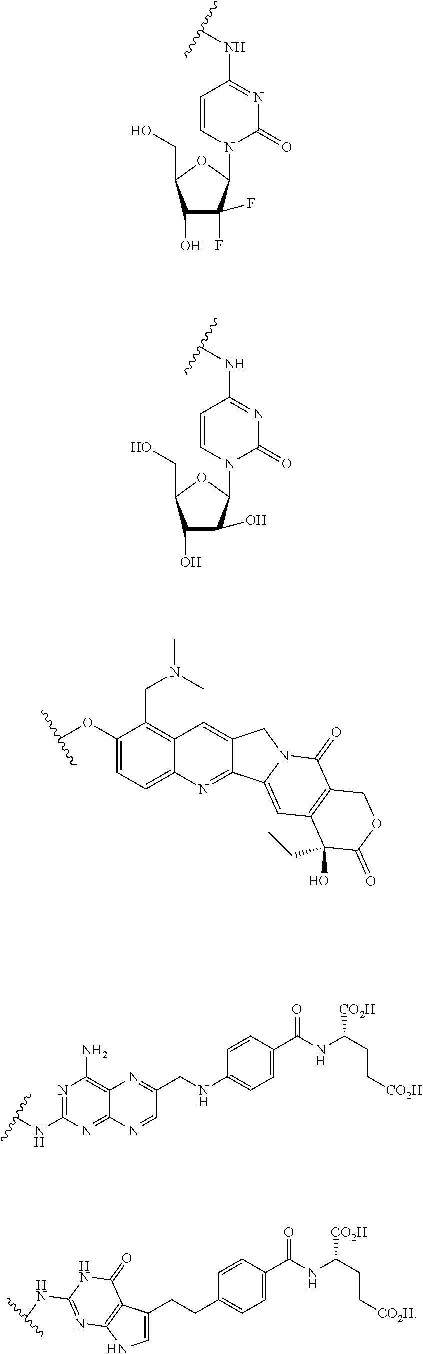

16. The compound of claim 1, wherein the compound has any one of Formulas 1001 to 1023:

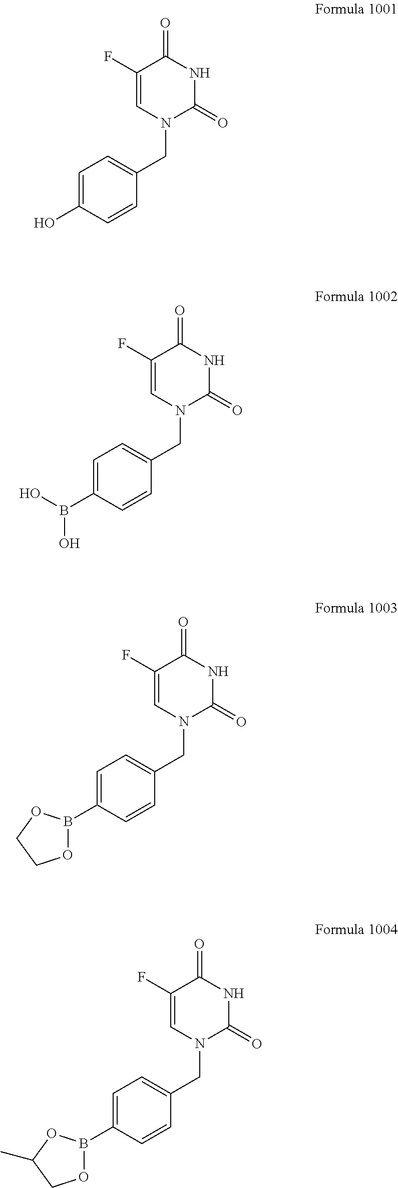 [SMILES: O=c1[nH]c(=O)n(Cc2ccc(O)cc2)cc1F ; O=c1[nH]c(=O)n(Cc2ccc(B(O)O)cc2)cc1F ; O=c1[nH]c(=O)n(Cc2ccc(B3OCCO3)cc2)cc1F ; CC1COB(c2ccc(Cn3cc(F)c(=O)[nH]c3=O)cc2)O1]

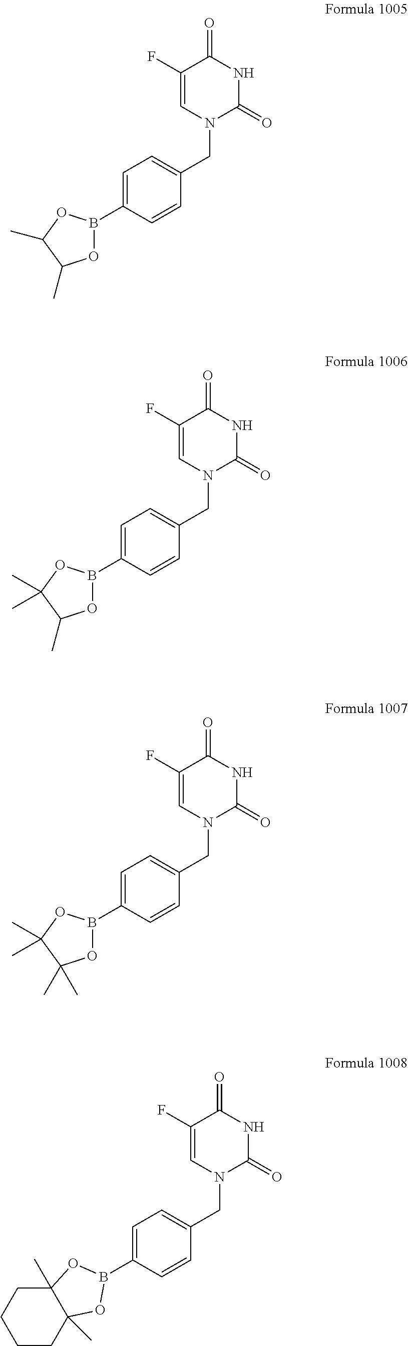 [SMILES: CC1OB(c2ccc(Cn3cc(F)c(=O)[nH]c3=O)cc2)OC1C ; CC1OB(c2ccc(Cn3cc(F)c(=O)[nH]c3=O)cc2)OC1(C)C ; CC1(C)OB(c2ccc(Cn3cc(F)c(=O)[nH]c3=O)cc2)OC1(C)C ; CC12CCCCC1(C)OB(c1ccc(Cn3cc(F)c(=O)[nH]c3=O)cc1)O2]

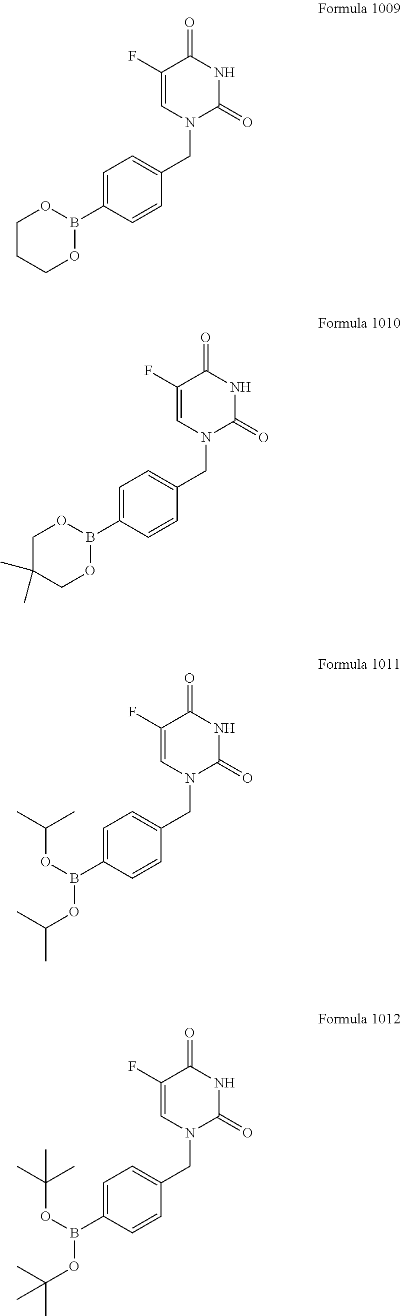 [SMILES: O=c1[nH]c(=O)n(Cc2ccc(B3OCCCO3)cc2)cc1F ; CC1(C)COB(c2ccc(Cn3cc(F)c(=O)[nH]c3=O)cc2)OC1 ; CC(C)OB(OC(C)C)c1ccc(Cn2cc(F)c(=O)[nH]c2=O)cc1 ; CC(C)(C)OB(OC(C)(C)C)c1ccc(Cn2cc(F)c(=O)[nH]c2=O)cc1]

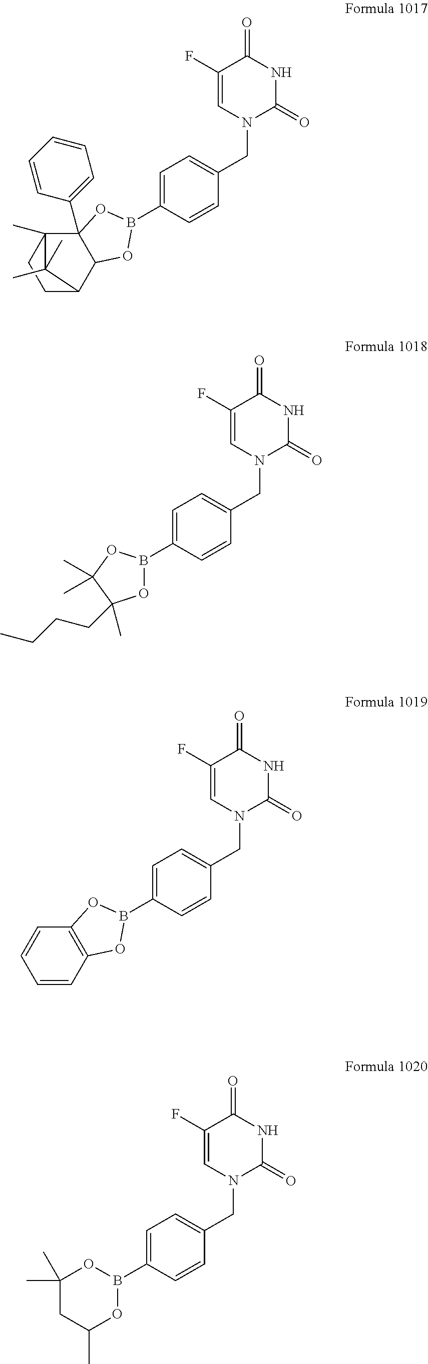 [SMILES: CC1(C)C2CCC1(C)C1(c3ccccc3)OB(c3ccc(Cn4cc(F)c(=O)[nH]c4=O)cc3)OC21 ; CCCCC1(C)OB(c2ccc(Cn3cc(F)c(=O)[nH]c3=O)cc2)OC1(C)C ; O=c1[nH]c(=O)n(Cc2ccc(B3Oc4ccccc4O3)cc2)cc1F ; CC1CC(C)(C)OB(c2ccc(Cn3cc(F)c(=O)[nH]c3=O)cc2)O1]

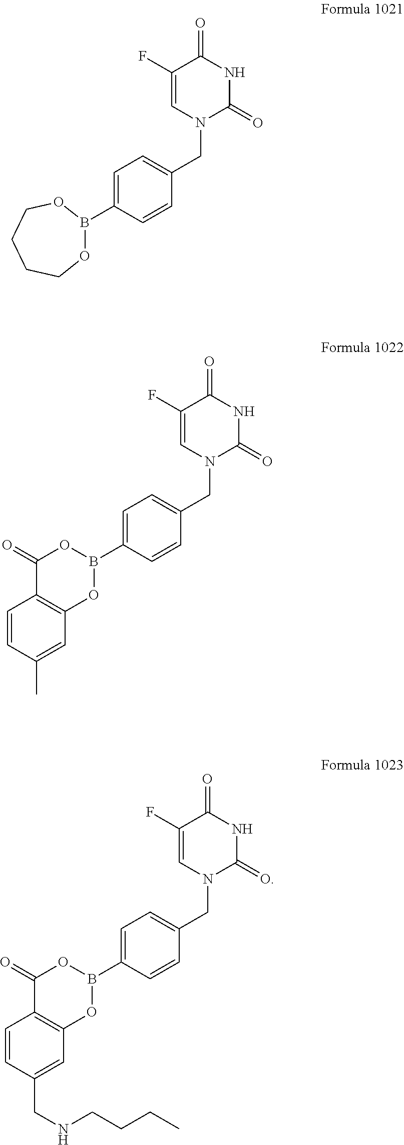 [SMILES: O=c1[nH]c(=O)n(Cc2ccc(B3OCCCCO3)cc2)cc1F ; Cc1ccc2c(c1)OB(c1ccc(Cn3cc(F)c(=O)[nH]c3=O)cc1)OC2=O ; CCCCNCc1ccc2c(c1)OB(c1ccc(Cn3cc(F)c(=O)[nH]c3=O)cc1)OC2=O]

17. The compound of claim 1, wherein the compound has Formula 2001, Formula 2002, or Formula 2003:

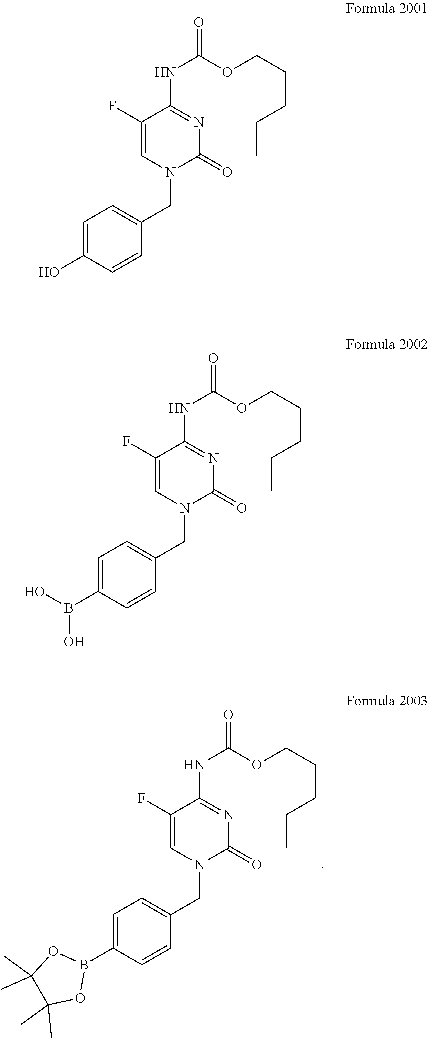 [SMILES: CCCCCOC(=O)Nc1nc(=O)n(Cc2ccc(O)cc2)cc1F ; CCCCCOC(=O)Nc1nc(=O)n(Cc2ccc(B(O)O)cc2)cc1F ; CCCCCOC(=O)Nc1nc(=O)n(Cc2ccc(B3OC(C)(C)C(C)(C)O3)cc2)cc1F]

18. The compound of claim 1, wherein the compound has any one of Formulas 1001-a to 1023-a:

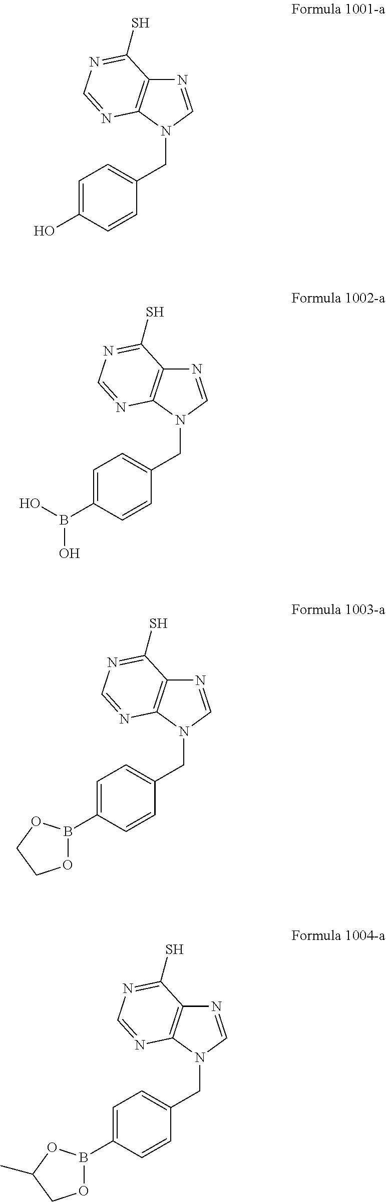 [SMILES: Oc1ccc(Cn2cnc3c(S)ncnc32)cc1 ; OB(O)c1ccc(Cn2cnc3c(S)ncnc32)cc1 ; Sc1ncnc2c1ncn2Cc1ccc(B2OCCO2)cc1 ; CC1COB(c2ccc(Cn3cnc4c(S)ncnc43)cc2)O1]

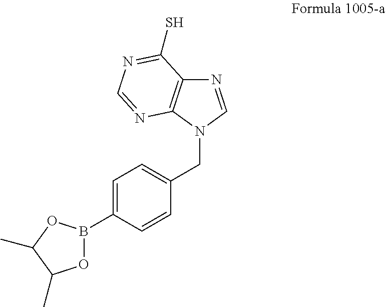 [SMILES: CC1OB(c2ccc(Cn3cnc4c(S)ncnc43)cc2)OC1C]

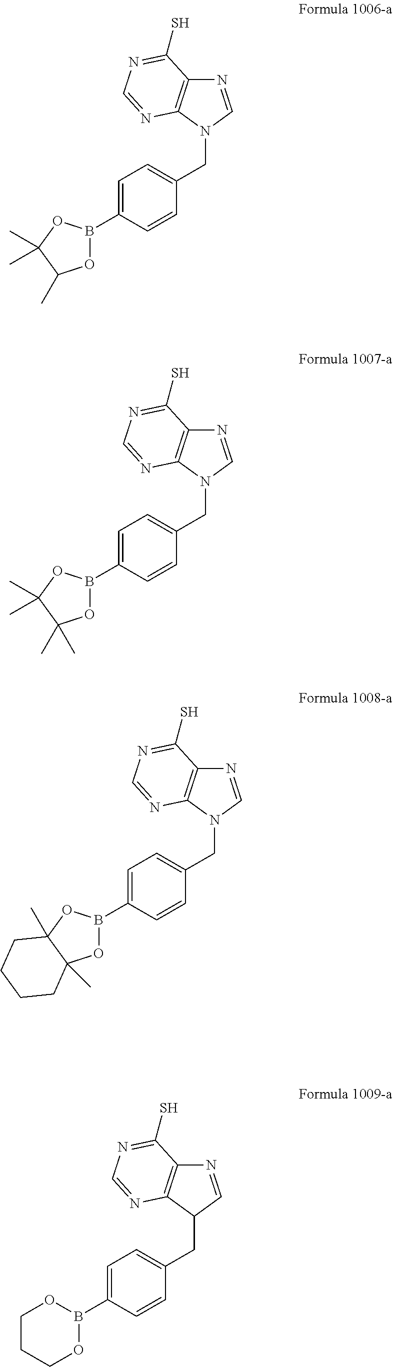 [SMILES: CC1OB(c2ccc(Cn3cnc4c(S)ncnc43)cc2)OC1(C)C ; CC1(C)OB(c2ccc(Cn3cnc4c(S)ncnc43)cc2)OC1(C)C ; CC12CCCCC1(C)OB(c1ccc(Cn3cnc4c(S)ncnc43)cc1)O2 ; Sc1ncnc2c1N=CC2Cc1ccc(B2OCCCO2)cc1]

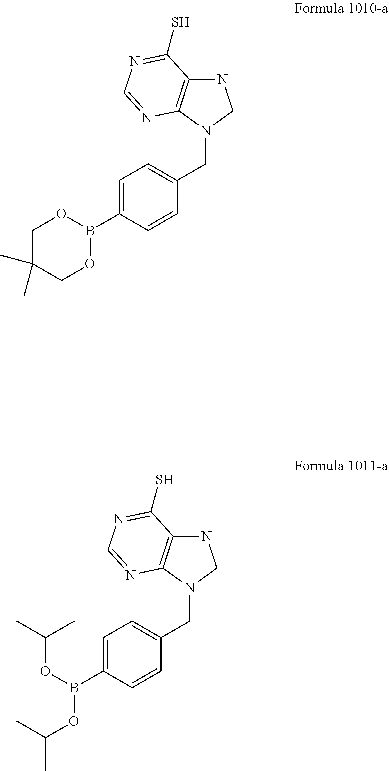 [SMILES: CC1(C)COB(c2ccc(CN3CNc4c(S)ncnc43)cc2)OC1 ; CC(C)OB(OC(C)C)c1ccc(CN2CNc3c(S)ncnc32)cc1]

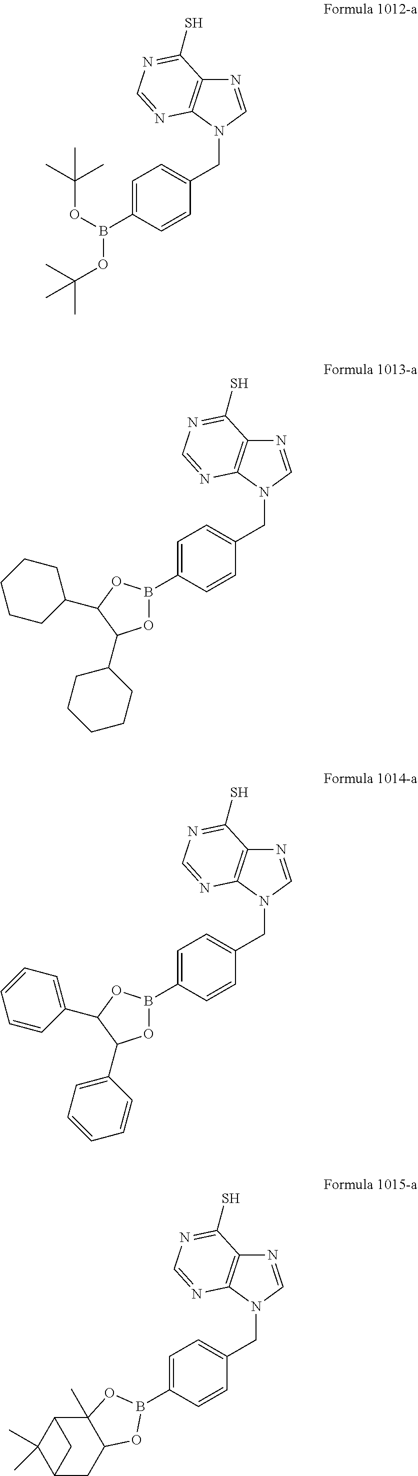 [SMILES: CC(C)(C)OB(OC(C)(C)C)c1ccc(Cn2cnc3c(S)ncnc32)cc1 ; Sc1ncnc2c1ncn2Cc1ccc(B2OC(C3CCCCC3)C(C3CCCCC3)O2)cc1 ; Sc1ncnc2c1ncn2Cc1ccc(B2OC(c3ccccc3)C(c3ccccc3)O2)cc1 ; CC1(C)C2CC3OB(c4ccc(Cn5cnc6c(S)ncnc65)cc4)OC3(C)C1C2]

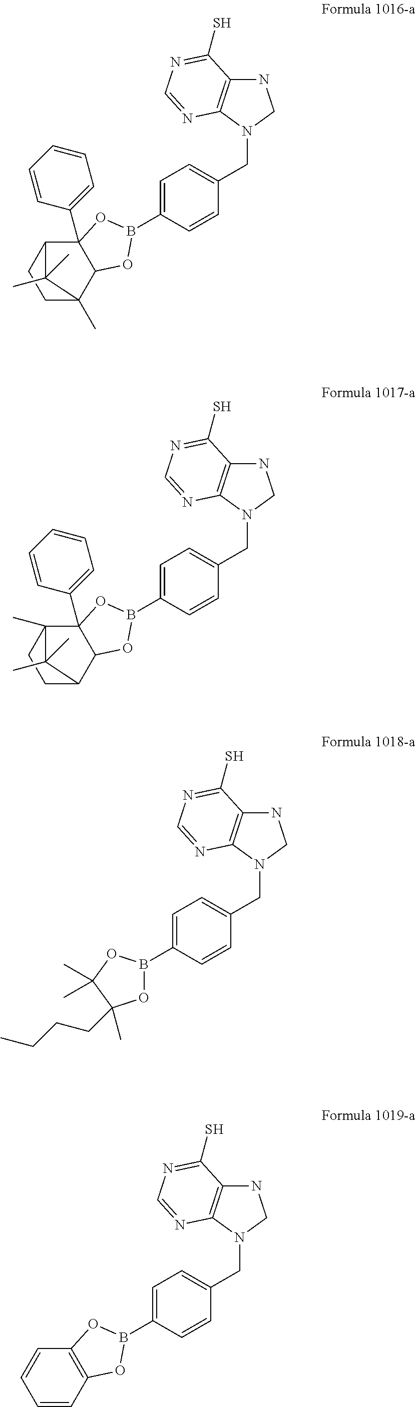 [SMILES: CC1(C)C2CCC1(C)C1OB(c3ccc(CN4CNc5c(S)ncnc54)cc3)OC21c1ccccc1 ; CC1(C)C2CCC1(C)C1(c3ccccc3)OB(c3ccc(CN4CNc5c(S)ncnc54)cc3)OC21 ; CCCCC1(C)OB(c2ccc(CN3CNc4c(S)ncnc43)cc2)OC1(C)C ; Sc1ncnc2c1NCN2Cc1ccc(B2Oc3ccccc3O2)cc1]

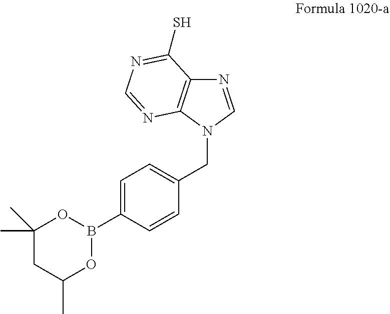 [SMILES: CC1CC(C)(C)OB(c2ccc(Cn3cnc4c(S)ncnc43)cc2)O1]

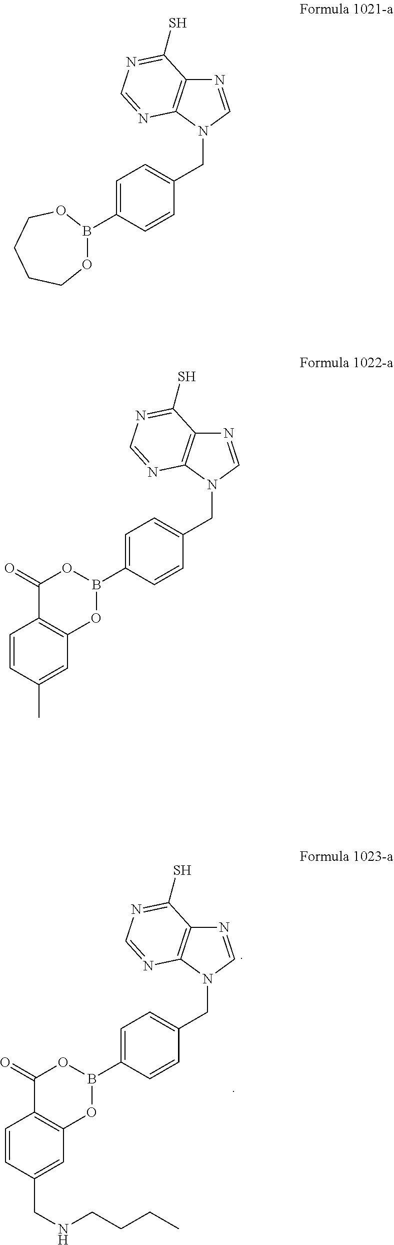 [SMILES: Sc1ncnc2c1ncn2Cc1ccc(B2OCCCCO2)cc1 ; Cc1ccc2c(c1)OB(c1ccc(Cn3cnc4c(S)ncnc43)cc1)OC2=O ; CCCCNCc1ccc2c(c1)OB(c1ccc(Cn3cnc4c(S)ncnc43)cc1)OC2=O]

19. The compound of claim 1, wherein the compound has any one of Formulas 1001-b to 1023-b:

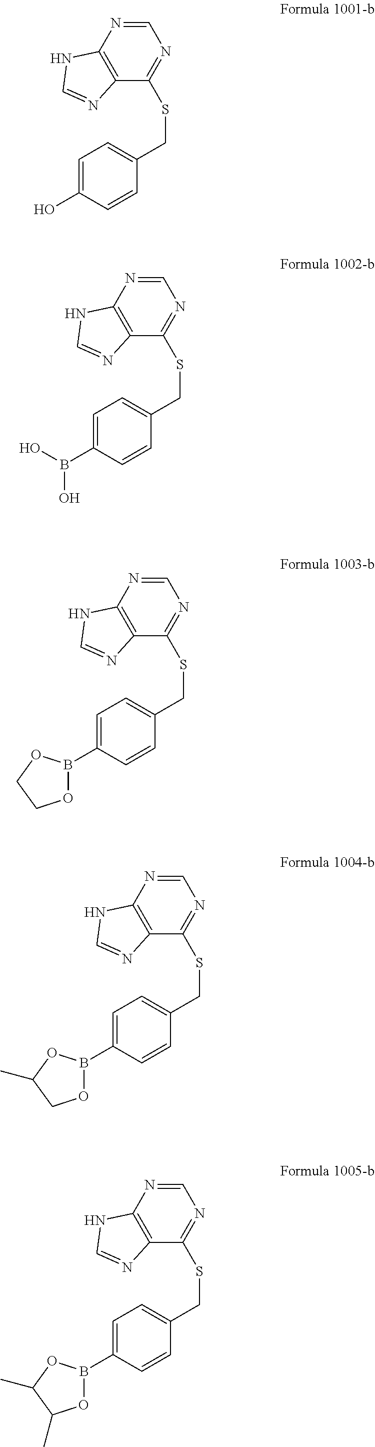 [SMILES: Oc1ccc(CSc2ncnc3[nH]cnc23)cc1 ; OB(O)c1ccc(CSc2ncnc3[nH]cnc23)cc1 ; c1nc(SCc2ccc(B3OCCO3)cc2)c2nc[nH]c2n1 ; CC1COB(c2ccc(CSc3ncnc4[nH]cnc34)cc2)O1 ; CC1OB(c2ccc(CSc3ncnc4[nH]cnc34)cc2)OC1C]

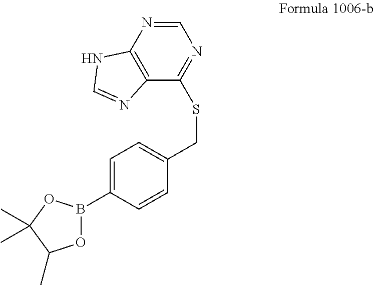 [SMILES: CC1OB(c2ccc(CSc3ncnc4[nH]cnc34)cc2)OC1(C)C]

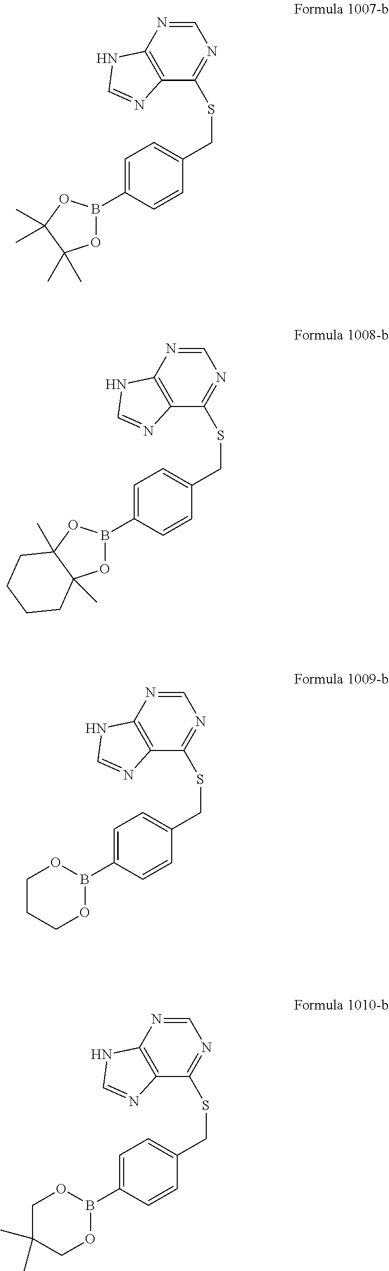 [SMILES: CC1(C)OB(c2ccc(CSc3ncnc4[nH]cnc34)cc2)OC1(C)C ; CC12CCCCC1(C)OB(c1ccc(CSc3ncnc4[nH]cnc34)cc1)O2 ; c1nc(SCc2ccc(B3OCCCO3)cc2)c2nc[nH]c2n1 ; CC1(C)COB(c2ccc(CSc3ncnc4[nH]cnc34)cc2)OC1]

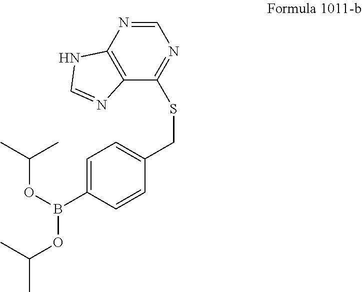 [SMILES: CC(C)OB(OC(C)C)c1ccc(CSc2ncnc3[nH]cnc23)cc1]

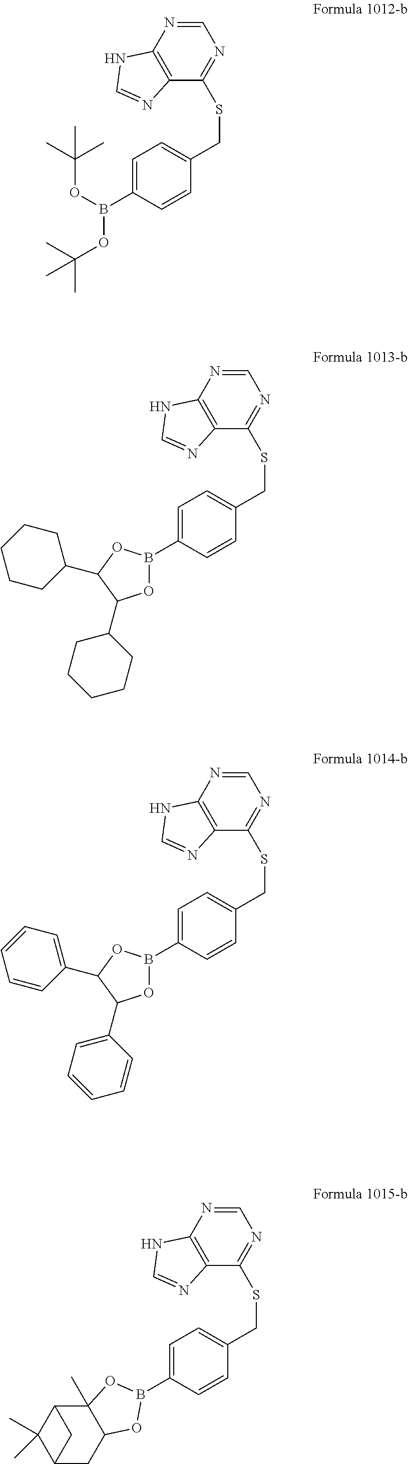 [SMILES: CC(C)(C)OB(OC(C)(C)C)c1ccc(CSc2ncnc3[nH]cnc23)cc1 ; c1nc(SCc2ccc(B3OC(C4CCCCC4)C(C4CCCCC4)O3)cc2)c2nc[nH]c2n1 ; c1ccc(C2OB(c3ccc(CSc4ncnc5[nH]cnc45)cc3)OC2c2ccccc2)cc1 ; CC1(C)C2CC3OB(c4ccc(CSc5ncnc6[nH]cnc56)cc4)OC3(C)C1C2]

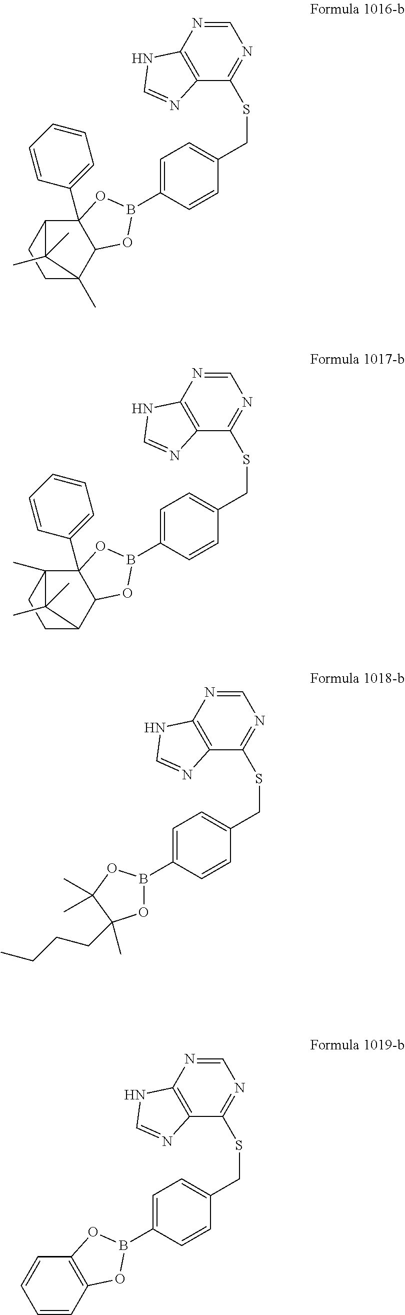 [SMILES: CC1(C)C2CCC1(C)C1OB(c3ccc(CSc4ncnc5[nH]cnc45)cc3)OC21c1ccccc1 ; CC1(C)C2CCC1(C)C1(c3ccccc3)OB(c3ccc(CSc4ncnc5[nH]cnc45)cc3)OC21 ; CCCCC1(C)OB(c2ccc(CSc3ncnc4[nH]cnc34)cc2)OC1(C)C ; c1ccc2c(c1)OB(c1ccc(CSc3ncnc4[nH]cnc34)cc1)O2]

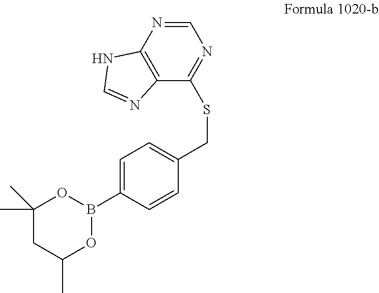 [SMILES: CC1CC(C)(C)OB(c2ccc(CSc3ncnc4[nH]cnc34)cc2)O1]

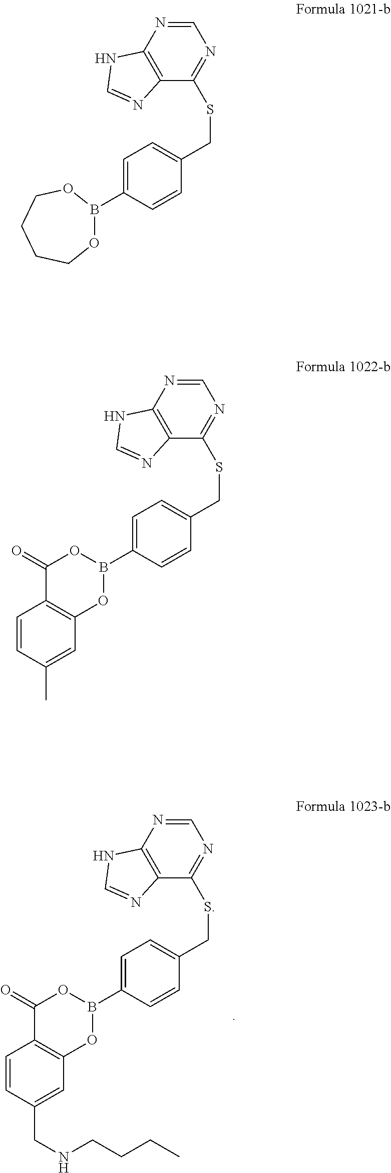 [SMILES: c1nc(SCc2ccc(B3OCCCCO3)cc2)c2nc[nH]c2n1 ; Cc1ccc2c(c1)OB(c1ccc(CSc3ncnc4[nH]cnc34)cc1)OC2=O ; CCCCNCc1ccc2c(c1)OB(c1ccc(CSc3ncnc4[nH]cnc34)cc1)OC2=O]

20. A pharmaceutical composition comprising a compound of claim 1, or a pharmaceutically acceptable salt, solvate, hydrate, cocrystal, or prodrug thereof, and a pharmaceutically acceptable carrier.

21. A method of treating a disease alleviated by anti-cancer or anti-autoimmune diseases chemotherapy in a patient in need thereof, comprising administering to the patient a therapeutically effective amount of a compound of claim 1, or a pharmaceutically acceptable salt, solvate, hydrate, cocrystal, or prodrug thereof.

22. A method of treating a disease alleviated by anti-cancer or anti-autoimmune diseases chemotherapy in a patient in need thereof, comprising administering to the patient the pharmaceutical composition of claim 20.

23.-29. (canceled)
